SEQUENCE LISTING (6 sequences)

SEQ ID NO: 1 - DNA, 30 bp, synthetic construct
  source 1..30: mol_type = other DNA
ggtcgccaccatggccgtcatggctccccg

SEQ ID NO: 2 - DNA, 30 bp, synthetic construct
  source 1..30: mol_type = other DNA
ggccgctttacactttacaagctgtgagag

SEQ ID NO: 3 - DNA, 30 bp, synthetic construct
  source 1..30: mol_type = other DNA
ttgtaaagtgtaaagcggccgcgtcgacaa

SEQ ID NO: 4 - DNA, 30 bp, synthetic construct
  source 1..30: mol_type = other DNA
tgacggccatggtggcgaccggtggatcct

SEQ ID NO: 5 - DNA, 21 bp, synthetic construct
  source 1..21: mol_type = other DNA
tgaaggcccactcacagactc

SEQ ID NO: 6 - DNA, 21 bp, synthetic construct
  source 1..21: mol_type = other DNA
cccacgtcgcagccatacatc

(19) United States
(12) Patent Application Publication
(10) Pub. No.: US 2025/0340846 A1
(43) Pub. Date: Nov. 6, 2025

(54) PATIENT-DERIVED CELL-CONTAINING DROPLETS ENABLE CLINICAL PRECISION ONCOLOGY

(71) Applicants: Xilis, Inc., Durham, NC (US); Duke University, Durham, NC (US)

(72) Inventor: Xiling Shen, Chapel Hill, NC (US)

(21) Appl. No.: 18/862,323

(22) PCT Filed: May 3, 2023
(86) PCT No.: PCT/US2023/066559

§ 371 Date: Nov. 1, 2024

Related U.S. Application Data
(60) Provisional application No. 63/338,022, filed on May 3, 2022.

(51) Int. Cl. C12N 5/09; C12N 15/86

(52) U.S. Cl. CPC C12N 5/0693; C12N 15/86; C12N 2503/00; C12N 2513/00; C12N 2740/15043

(57) ABSTRACT

Methods and materials for generating and using patient-derived MicroOrganoSpheres (e.g., MicroOrganoSpheres derived from tumor tissue) are provided herein.

### CROSS-REFERENCE TO RELATED APPLICATIONS

This application claims priority from U.S. Provisional Application Ser. No. 63/338,022, filed May 3, 2022. The disclosure of the prior application is considered part of (and is incorporated by reference in) the disclosure of this application.

### SEQUENCE LISTING

This application contains a Sequence Listing that has been submitted electronically as an XML file named “53157-0008WO1.XML.” The XML file, created on May 3, 2023, is 6,345 bytes in size. The material in the XML file is hereby incorporated by reference in its entirety.

### TECHNICAL FIELD

This document relates to methods and materials for generating and using patient-derived MicroOrganoSpheres.

### BACKGROUND

The success of precision oncology relies on models that capture the morphological, molecular, and functional characteristics of patient tumors to accurately predict drug response and resistance. The development of various patient-derived models of cancer (PDMC) has provided tools in this effort. For example, drug sensitivity assays using PDMC have recapitulated antitumor response in the clinic, underscoring their potential for guiding personalized care (Barretina et al., Nature 483, 603-607, 2012; Gao et al., Nature Med 21, 1318-1325, 2015; Lu et al., PLOS One 12, e0169439, 2017; Vlachogiannis et al., Science 359, 920-926, 2018). Patient-derived xenografts (PDX) and organoids (PDO) also have been shown to model clinical response to cancer therapy (Bruna et al., Cell 167, 260-274. e222, 2016; Gao et al., Nature Med 21, 1318-1325, 2015; Hidalgo et al., Cancer Discov 4, 998-1013, 2014; Jenkins et al., Cancer Discov 4, 998-1013, 2018; Neal et al., Cell 175, 1972-1988 e1916, 2018; Yuki et al., Trends Immunol 41, 652-664, 2020). Further, given the growing clinical importance of immuno-oncology (IO), there is significant interest to reproduce physiological immune activity in organoid cultures. For example, peripheral blood lymphocyte and tumor organoid co-culture models have been used to test tumor-reactive T cells (Dijkstra et al., Cell 174, 1586-1598, 2018). However, it can be challenging to use PDX and PDO models to guide timely clinical decisions for cancer patients.

### SUMMARY

As described herein, droplet emulsion microfluidics with temperature control and dead-volume minimization can be used to rapidly generate thousands of MicroOrganoSpheres (MOS) from low-volume patient tissues; the MOS can be highly useful as patient-derived models for clinical precision oncology. A clinical study of newly diagnosed metastatic colorectal cancer (CRC) patients using a MOS-based precision oncology pipeline reliably predicted patient treatment outcome within 14 days, a timeline suitable for guiding treatment decisions in clinic. Moreover, as described herein, MOS preserved stromal cells of the original tumor tissue and allowed T cell penetration, providing a clinical assay for testing IO therapies such as PD-1 blockade, bispecific antibodies, and T cell therapies on patient tumors.

In a first aspect, this document features a method that includes, or consists essentially of, obtaining a plurality of cells derived from a tissue; forming MicroOrganoSpheres (MOS) from the plurality of cells; culturing the MOS in a MOS culture; and introducing a virus into the MOS culture, thereby obtaining one or more cells infected with the virus in the MOS. The one or more infected cells can express one or more genes introduced by the virus after infection with the virus. The MOS can have an average diameter of about 50 μm to about 500 μm. In some cases, the plurality of cells includes no more than 15,000 cells. The method cells can be derived from a biopsy. The cells can be derived from a tumor biopsy. The cells can be derived from one or more core biopsies comprising from about a 14-gauge core to about a 20-gauge core biopsy. The cells can be derived from one or more 18-gauge core biopsies. The cells can be derived from a tumor biopsy for one or more cancers. The one or more cancers can include rectal cancer, lung cancer, breast cancer, colorectal cancer (CRC), kidney cancer, ovarian cancer, or combinations thereof. The cells can be derived from one or more patients. The cells can include CRC patient-derived xenograft (PDX) cells. The MOS can contain tumorspheres. The MOS can be cultured in droplets, where nascent MOS include a seeding density of about 1 to about 300 cells per droplet. The nascent MOS can have a seeding density configured to generate tumorspheres in the MOS of a desired quantity, size, or both. The MOS can be cultured in droplets, and the method can further include determining a number of MOS (NMOS) by dividing a number of viable cells by a number of cells per droplet. The method can further include treating the MOS with one or more therapeutic agents. The one or more therapeutic agents can include a small molecule or an antibody. The cells can be from a patient, and the MOS can function as a predictive model of the patient's sensitivity to one or more drug therapies for treating a disease. The MOS can function as a predictive model of the patient's sensitivity to one or more chemotherapies.

In another aspect, this document features a method that includes, or consists essentially of, obtaining a plurality of cells derived from tissue, mixing the plurality of cells with a fluid comprising a polymer, and intersecting a stream of the cells and fluid with a stream of immiscible material to generate a plurality of MicroOrganoSpheres (MOS). The method can further include demulsifying the generated MOS and/or culturing the generated MOS. In some cases, the method can include culturing the generated MOS as suspension droplets. The polymer can be a polymer matrix (e.g., an extracellular matrix). The MOS can have an average diameter of about 10 μm to about 700 μm. The MOS can have an average diameter configured to provide a three-dimensional cellular environment. In some cases, the plurality of cells may include no more than 15,000 cells, no more than 10,000 cells, no more than 5,000 cells, or no more than 1,000 cells. In some cases, the plurality of cells can include from about 50 cells to about 20,000 cells (e.g., from about 500 cells to about 10,000 cells). The cells can be derived from a biopsy (e.g., a tumor biopsy). The cells can be derived from one or more core biopsies (e.g., one or more biopsies having about a 14-gauge core to about a 20-gauge core biopsy). The cells can be derived from one or more 18-gauge core biopsies. The cells can be derived from a tumor biopsy for one or more cancers. The one or more cancers can include rectal cancer, lung cancer, breast cancer, colorectal cancer (CRC), kidney cancer, ovarian cancer, or combinations thereof. The cells can be derived from one or more patients. The cells can include CRC patient-derived xenograft (PDX) cells. The MOS can include tumorspheres and/or tumorsphere-like structures in the presence of tumor-resident immune cells. The mixing can form a plurality of nascent MOS that subsequently form the MOS. The nascent MOS can include a seeding density of about 20 to about 100 cells per droplet, about 20 to about 50 cells per droplet, about 30 to about 70 cells per droplet, about 40 to about 60 cells per droplet, or about 50 to about 100 cells per droplet. The nascent MOS can include a seeding density configured to generate tumorspheres in the MOS of a desired quantity, size, or both. The method can further include determining a number of MOS (NMOS) by dividing a number of viable cells by a number of cells per droplet. The method can further include treating the MOS with one or more therapeutic agents. The one or more therapeutic agents can include a small molecule or an antibody. The therapeutic agent can be any chemotherapeutic agent. The treating can include delivering one or more therapeutic agents at a concentration from about 1 μM to about 10 μM. The one or more therapeutic agents can include oxaliplatin, irinotecan, or a combination thereof. The treating can occur less than 11 days after a biopsy acquisition, less than 5 days after a biopsy acquisition, or less than 3 days after a biopsy acquisition. Each MOS can contain at least 30 tumor cells, at least 20 tumor cells, or at least 10 tumor cells. In some cases, each MOS can contain from about 10 tumor cells to about 50 tumor cells. The MOS can function as a predictive model of a patient's sensitivity to one or more drug therapies for treating a disease. The MOS can function as a predictive model of a patient's sensitivity to one or more chemotherapies. The MOS can function as a predictive model of a patient's sensitivity to one or more chemotherapies within 14 days of MOS preparation. The MOS can contain an amount of fibroblasts that is less than that found in comparative bulk organoid cultures. For example, the amount of fibroblasts in the MOS can be less than that found in comparative bulk organoid cultures after 2 days of culturing, less than that found in comparative bulk organoid cultures after 5 days of culturing, or less than that found in comparative bulk organoid cultures after 7 days of culturing. The MOS can contain functional immune cells. The MOS can contain immune cells that are responsive to an immune therapy. The MOS can contain natural killer cell markers (e.g., CD4+, CD8+, CD56+, or a combination thereof).

In another aspect, this document features a method of predicting a patient's response to a therapeutic treatment. The method can include, or consist essentially of, co-culturing Patient-Derived MicroOrganoSpheres (MOS) with an agent associated with an immune therapy; and assaying the MOS to determine potency of the immune therapy. The immune therapy can be immune-oncology (IO) therapy. The agent can include an immune checkpoint inhibitor, a T cell activator, tumor infiltrating lymphocytes (TILs), an IO therapy molecule, or a combination thereof. The immune checkpoint inhibitor can be an anti-PD1 therapy (e.g., nivolumab, pembrolizumab, cemiplimab, atezolizumab, dostarlimab, durvalumab, or avelumab). The IO therapy molecule can include a PD-1 blockade, T-cell bispecific antibody (TCB), or both. The immune therapy can target a human leukocyte antigen (HLA), antigens associated with the HLA, or both. The agent can include a T-cell receptor-mimic antibody (e.g., ESK1, DP47, or both). The agent can be present in an amount of about 0.1 μg/mL to about 10 μg/mL, about 0.5 g/mL to about 5 μg/mL, or about 1 μg/mL to about 3 μg/mL. The method can include determining an amount of cell apoptosis that occurred in tumorspheres present within the MOS following initiation of the immune therapy. The MOS can function as a predictive model for at least 12 months, at least 6 months, or at least 3 months.

In another aspect, this document features a method of treating a patient. The method can include, or consist essentially of, (a) predicting a patient response to a therapeutic treatment as described herein; and (b) selecting a therapy based on the predicted patient response.

In another aspect, this document features a method for predicting a patient's response to a therapy. The method can include, or consist essentially of, (a) co-culturing Patient-Derived MicroOrganoSpheres (MOS) with effector immune cells; and (b) assaying the MOS to determine potency of the therapy with the effector immune cells. The immune cells can be selected from the group consisting of chimeric antigen receptor (CAR) T cells, tumor infiltrating lymphocytes (TILs), peripheral blood mononuclear cells (PBMCs), T cells isolated from PBMCs, T cells isolated and expanded from tumor cells, and combinations thereof. The MOS can be formed by a method described herein.

In still another aspect, this document features a MicroOrganoSphere composition. The composition can include, consist essentially of, or consist of a plurality of MicroOrganoSpheres, with each MicroOrganoSphere including a base material and at least one tumorsphere, wherein the plurality of MicroOrganoSpheres contains a predetermined number of cells per droplet, a predetermined number of droplets in the composition, and/or a predetermined droplet size. The composition can further include one or more drug therapies. The at least one tumorsphere can be responsive to one or more drug therapies.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1J: Establishing CRC MOS for drug screen and clinical validation. FIG. 1A depicts a scheme for CRC MOS generation and drug screening. FIG. 1B includes images of a microfluidic MOS chip. FIG. 1C includes bright field microscope images of CRC MOS generated with different cell numbers per MOS. FIG. 1D includes representative images of generated MOS from patient CRC tumor tissue and hematoxylin and eosin (H&E) staining of the primary CRC tumor tissue and derived MOS. FIG. 1E includes images showing H&E staining of CRC MOS established from different patient tumor tissues. FIG. 1F is a heat map of high throughput drug screen using CRC tumor-derived MOS indicates sensitivity to oxaliplatin and resistance to Irinotecan. FIG. 1G includes images showing patient response to oxaliplatin after 6 months of treatment in clinic. FIG. 1H is a schematic illustration of a clinical study design for using MOS established from CRC biopsy for drug testing. FIG. 1I includes representative images of patient-derived MOS. FIG. 1J is a Kaplan Meier graph plotting survival outcomes, showing that survival of eight CRC patients was correlated with MOS drug sensitivity. Scale bar: 100 μm.

FIGS. 2A-2H: CRC clinical study. FIG. 2A is an image of a MOS generation machine and associated microfluidics system (100). A 15 mL conical tube containing an immiscible fluid (e.g., oil) (110) and a 1.5 mL Eppendorf tube kept on ice containing the cell/MATRIGEL® sample mixture (120) are pressurized to drive flow through the microfluidic chip found inside the chip holder (130). The device is placed in a refrigerator with tubes connected to pumps (140) on the outside. FIGS. 2B-2H include graphs plotting the results of in vitro CRC MOS drug responses and images showing clinical validation from seven different CRC patients. n=3 for each drug concentration.

FIGS. 3A-3F: Oxaliplatin drug treatment of CRC MOS and phenotypic characterization of cancer patient-derived MOS. FIG. 3A includes representative images of CRC MOS derived from two patients (designated as CRC1282 and CRC1297) treated with oxaliplatin. FIG. 3B includes scatter plots of the caspase 3/7 fluorescence signal in the CRC MOS, normalized by surface area of the individual tumorspheres inside MOS. FIG. 3C includes white light images of MOS generated from patient breast tumor tissue, as well as images showing H&E staining of the primary breast tumor tissue and derived MOS. Scale bar: 100 μm. FIG. 3D includes white light images of MOS generated from patient kidney tumor tissue, as well as images showing H&E staining of the primary kidney tumor tissue and derived MOS. Scale bar: 100 μm. FIG. 3E includes graphs plotting data to compare tumorspheres growth in MOS vs. organoids in bulk MATRIGEL® FIG. 3F includes representative images of day 7 MOS vs. traditional MATRIGEL®, suggesting less fibroblast growth in the MOS than in the MATRIGEL®.

FIGS. 4A-4E: Genomic and transcriptomic characterization of MOS generated from patient lung tumor. FIG. 4A includes representative images showing MOS generated from patient lung tumor tissue, along with images showing H&E staining of the primary lung tumor tissue and derived MOS. FIG. 4B includes copy number variation (CNV) profiles showing correlations of lung tumor tissue and derived MOS. FIG. 4C includes UMAPs of cells from primary lung tumor tissue and derived MOS labeled by cell types. FIG. 4D includes graphs plotting a comparison of log-transformed relative abundance of each cell type for three lung tumor samples and derived MOS. FIG. 4E is a graph plotting the relative abundance of cell types represented in tissue (n=3) or MOS (n=3) samples, as indicated. Abundance is reported as log 1p (percentage out of 1).

FIGS. 5A-5E: Characterization of cancer patient-derived MOS. FIG. 5A includes flow cytometry plots showing that MOS support fewer Vimentin (+) fibroblasts than MATRIGEL® FIG. 5B shows copy number variation (CNV) profiles with correlations of breast, kidney, and ovarian tumor tissues and MOS derived therefrom. FIG. 5C includes clustermaps of Jaccard similarity scores between mutation profiles for each sample and disease state. FIG. 5D includes Venn diagrams showing the fraction of shared mutations between tumors (T) and matched MOS (M). FIG. 5E is a graph plotting Driver mutation analysis in tumor and its derived MOS.

FIGS. 6A-6F: Single cell RNA-seq analysis of patient tissue and derived MOS. FIG. 6A shows the results of quality assessment metrics plotted as a UMAP for all cells. FIG. 6B is a pair of violin plots of a quality assessment of cells profiled using Drop-seq. nCount_RNA and nFeature_RNA describe the distribution of the number of sequencing reads or observed genes associated with cells profiled in each sample, respectively. Cells with more than 2,500 observed genes were removed from downstream analysis. FIG. 6C shows cells from three lung tumor tissue samples and their derived MOS samples, plotted as a UMAP labelled by cell types. FIG. 6D includes plots showing cells from primary kidney tumor tissue and MOS samples plotted as a UMAP labeled by cell type (upper left) and preparation (upper right), and log-transformed relative abundance of cell types in the kidney samples and derived MOS (bottom). FIG. 6E includes plots showing cells from primary ovarian tumor tissue and MOS samples plotted as a UMAP labeled by cell type (upper left) and preparation (upper right), and log-transformed relative abundance of cell types in the ovarian samples and derived MOS (bottom). FIG. 6F includes plots showing cells from primary CRC tissue and MOS samples plotted as a UMAP labeled by cell type (upper left) and preparation (upper right), and a graph plotting relevant abundance of four major cell types in the CRC tissue and derived MOS at day 7 (bottom).

FIGS. 7A-7D: Gene expression analysis in tumor tissue vs. derived MOS. FIG. 7A includes flow cytometry plots showing the characterization of CRC MOS stroma, and demonstrating that key immune cell populations were preserved in MOS. FIG. 7B is a plot comparing the log-fold change in gene expression for tumor cells, fibroblasts, lymphocytes, and myeloid cells from a kidney cancer sample (n=1). The gene with absolute log-fold change >1.5 is labeled. FIG. 7C is a plot comparing the log-fold change in gene expression for tumor cells, fibroblasts, lymphocytes, and myeloid cells from an ovarian cancer sample (n=1). Genes with absolute log-fold change >1.5 are labeled. FIG. 7D includes a series of UMAPs plotting the expression of broad cell type marker genes for each of the broad cell types in lung samples and derived MOS. Tumor cell markers included EPCAM and CDH1. Myeloid cell markers included FCERIA and LYZ. Lymphocyte markers included CD3E and IL7R. Fibroblast cell markers included FAP and PDGFRA.

FIGS. 8A-8D: Differential gene analysis on lung tumor tissue vs. derived MOS. FIG. 8A is a plot for a pseudo-bulk differential expression analysis comparing lung cancer primary tissue and derived MOS datasets (n=3). Genes with absolute log-fold change >1.5 are labeled. FIG. 8B includes volcano plots of differentially expressed genes from epithelial cells, lymphoid cells, myeloid cells, and fibroblasts from lung tumor samples. FIG. 8C includes UMAPs plotting expression of cancer-associated marker genes CD274 (PD-L1), PDCD1 (PD-1), and TGFB1 (TGF-beta). Cells are plotted on separate UMAPs depending on source: primary tissue (left) or MOS (right). FIG. 8D is a graph plotting the top five identified conserved markers for each cell type, labeled by cell source.

FIGS. 9A-9M: MOS in response to immunotherapy. FIG. 9A includes representative images and flow cytometry plots showing that resident immune cells encapsulated in MOS are viable and responsive to immune stimulation. FIG. 9B is an image showing that kidney tumor MOS were established on day 3. FIG. 9C is a graph showing that Nivolumab (10 μg/mL) induces kidney tumor MOS killing (indicated by Annexin V). FIG. 9D includes representative images from Incucyte showing death of tumorspheres within MOS in response to Nivolumab treatment vs. control. FIG. 9E is a graph showing that ESK1*(10 μg/mL) induces more death in lung tumor MOS than 1 μg/mL ESK1*. FIG. 9F includes representative images from Incucyte suggesting that a higher dose of ESK1* induces more killing (higher Annexin V signals). FIG. 9G is a graph plotting the level of cell death, showing that CRC tumorspheres in MOS are responsive to ESK1* treatment. Annexin V fluorescence signal from each organoid was measured 3 days after drug dosing. Each dot represents an individual organoid. FIG. 9H is a graph plotting the level of cell death, showing that CRC organoids in MATRIGEL® dome do not respond to ESK1* drug treatment. Annexin V fluorescence signal from each organoid was measured 3 days after drug dosing. Each dot represents an individual organoid. FIG. 9I includes representative images of tumorspheres in MOS and organoids in MATRIGEL® dome on day 3 after ESK1* treatment. FIG. 9J is a graph showing that ESK1* induced lung tumor MOS killing (indicated by Annexin V) compared to DP47 (CD3 only TCB). FIG. 9K is a UMAP of cells from primary lung tumor tissue and three MOS samples treated with ESK1*, negative TCB, or drug. FIG. 9L is a UMAP with cells indicated by sample source. FIG. 9M includes UMAPs of cells from primary lung tumor tissue and derived MOS, with and without treatments.

FIGS. 10A-10I: Immune cells preserved in MOS are responsive to immunotherapy. FIG. 10A is a graph showing that Nivolumab induced significant cytotoxicity in tumorspheres within MOS. Incucyte images were taken every 2 hours for 4 days, and Annexin V Green dye was added to indicate apoptosis. FIG. 10B includes representative Incucyte images demonstrating that Nivolumab induces cell apoptosis within MOS. FIG. 10C is an image of established MOS (day 4) derived from lung tumor tissue. FIG. 10D is a schematic depicting how ESK1* TCB drug induces CTL-mediated killing in MOS. FIG. 10E is a graph plotting the level of HLA-A2 gene expression in lung tumor tissues. FIG. 10F is a flow cytometry plot showing HLA-A2 expression in established MOS derived from lung tumor tissue.

FIG. 10G is a graph showing that ESK1* induced a higher apoptosis signal than DP47 (indicated by Annexin V signal) in MOS. FIG. 10H includes representative images showing apoptosis induced by ESK1* treatment of MOS. FIG. 10I is a plot showing that ESK1* induced killing of lung cancer MOS in all eight lung cases (p<0.005).

FIGS. 11A-11O: A MOS potency assay for T-Cell therapies. FIG. 11A is an image showing TILs and traditional MATRIGEL®, showing that TILs cannot penetrate traditional MATRIGEL®. Immune cells were stained with Cytolight Red dye before the image was taken using Incucyte. FIG. 11B includes images showing that TILs can penetrate MOS and adhere to tumor cells. Immune cells were stained with Cytolight Red dye before the images were taken using Incucyte. FIG. 11C is a graph showing that increased killing (indicated by Annexin V) was observed in MOS treated with autologous TILs. FIG. 11D includes representative images showing MOS killing by TILs (indicated by Annexin V dye). FIG. 11E is a graph showing that activated PBMCs induce MOS killing (indicated by Annexin V Green dye). FIG. 11F includes representative images showing MOS killing by PBMCs. FIG. 11G includes representative images illustrating an imaging analysis pipeline that identifies droplet area to minimize background noise from outside immune cells. FIG. 11H is a graph from a quantification analysis suggesting that PBMCs induce MOS killing (indicated by Caspase 3/7 dye). FIG. 11I is a graph showing that ESK1* enhanced PBMC-induced tumor cell killing compared to DP47 (CD3 only TCB). FIG. 11J includes representative images showing induced death of ESK1*-treated MOS combined with PBMCs. White arrows indicate lung cancer tumorspheres within MOS. Compared to ESK1*, the negative control TCB, DP47, did not induce significant apoptosis of tumorspheres within MOS. FIG. 11K is a dot plot indicating that ESK1* induced PBMC-mediated lung tumor MOS death in seven patient cases (p<0.005). FIG. 11L is a representative image showing lung tumor-derived MOS infected with a dsRed expressing vector (shown 3 days post infection). FIG. 11M is a graph plotting HLA-A2 expression in virus-treated samples, showing that significantly higher gene expression of HLA-A2 was observed in HLA-A2-infected MOS. FIG. 11N includes flow plots showing that significantly higher antigen expression was observed in HLA-A2-infected MOS. FIG. 11O is a graph showing that HLA-A2-infected MOS underwent higher cell death than matched uninfected MOS in the presence of ESK1* and activated PBMCs (as indicated by Annexin V dye).

FIGS. 12A-12M: MOS-based T cell potency assay for lung, kidney and colorectal cancer. FIG. 12A includes representative images showing PBMC penetration of MOS MATRIGEL®. Immune cells were stained with Cytolight Rapid Red. Images were taken every 2 hours for 3 days by Incucyte. FIG. 12B includes representative images showing that activated PBMCs induced MOS killing. Tumorspheres inside MOS were stained with Cytolight Red dye to indicate cell viability, FIG. 12C includes representative images showing that activated PBMCs induced MOS death indicated by Caspase3/7 Green dye. FIG. 12D includes representative images showing that activated PBMCs induced MOS death indicated by Cytotox Green dye. FIG. 12E is a graph showing that pre-activated allogenic PBMCs induce CRC MOS death (E: T=5:1), as indicated by Caspase 3/7 green dye. FIG. 12F includes representative Incucyte images indicating significant killing in CRC MOS combined with activated PBMC (E: T=5:1). FIG. 12G is a graph showing that pre-activated allogenic PBMCs induce kidney tumor MOS death, as shown by Caspase3/7 signal. FIG. 12H includes representative Incucyte images suggesting a higher level of death in kidney tumor MOS combined with pre-activated PBMC at an effector: target ratio of 10:1 than at a ratio of 5:1. FIG. 12I is a graph plotting the effects of Nivolumab (PD-1 blockade) treatment on lung tumor MOS with and w/o matched patient TILs and MHC blockade. FIG. 12J includes representative images showing that ESK1* enhanced TIL-induced killing of lung tumor MOS as compared to DP47 (CD3 only TCB). FIG. 12K includes representative images showing the heterogeneity of cell death in ESK1*/TIL-treated MOS at various times (killing indicated by the circles). FIG. 12L is a graph plotting ESK1*-induced tumorsphere death in each lung tumor MOS. FIG. 12M is an illustration of a HLA-A2 vector map.

### DETAILED DESCRIPTION

This document provides methods and materials that can be used to generate and use MOS. For example, as described herein, droplet emulsion microfluidics with temperature control and dead-volume minimization can be used to rapidly generate thousands of MOS from low-volume patient tissues (e.g., tumor biopsies). The MOS can serve as patient-derived models for clinical precision oncology, predicting patient response to particular therapeutic agents and predicting treatment outcome within 14 days—a timeline suitable for guiding treatment decisions in clinic. Moreover, since MOS have now been determined to contain original tumor-derived stromal cells that permit T cell penetration and, as described herein, have been demonstrated to contain tumor-derived immune cells in an environment that effectively mimics that of the original tumor, the MOS provide a clinical assay for testing IO therapies such as checkpoint inhibitors (e.g., PD-1 blockade), bispecific antibodies, and T cell therapies on patient tumors.

In some cases, this document provides methods for generating MOS. In some variations, the MOS are formed by forming a droplet of the unpolymerized mixture of a dissociated tissue sample and a fluid matrix material in an immiscible material, such as a fluid hydrophobic material (e.g., oil). For example, MOS may be formed by combining a stream of unpolymerized material that contains cells of a dissociated tissue sample with one or more streams of the immiscible material to form a droplet. In some cases, MOS can be formed according to one or more of the methods described in U.S. Pat. No. 11,555,180, which is incorporated herein by reference in its entirety. See, for example, column 3, line 5 to column 7, line 5, and column 21, line 54 to column 22, line 57. In some cases, the method also can include demulsifying and/or culturing the generated MOS. For example, the MOS can be cultured as droplets. In some cases, the MOS can be cultured as suspension droplets.

Any suitable polymer and immiscible fluid (e.g., oil) can be used. In some cases, for example, the polymer can be a polymer matrix (e.g., an extracellular matrix, such as a MATRIGEL® matrix).

The MOS can have any suitable diameter. For example, the MOS can have an average diameter of about 10 μm to about 700 μm (e.g., about 10 to about 50 μm, about 50 to about 100 μm, about 100 to about 150 μm, about 150 to about 200 μm, about 200 to about 250 μm, about 250 to about 300 μm, about 300 to about 350 μm, about 350 to about 400 μm, about 400 to about 450 μm, about 450 to about 500 μm, about 500 to about 550 μm, about 550 to about 600 μm, about 600 to about 650 μm, or about 650 to about 700 μm). In some cases, the MOS in a population can have an average diameter configured to provide a three-dimensional cellular environment. In some cases, the plurality of cells may include no more than 15,000 cells (e.g., no more than 10,000 cells, no more than 5,000 cells, or no more than 1,000 cells). In some cases, the plurality of cells can include from about 100 cells to about 20,000 cells (e.g., from about 100 to about 500 cells, from about 500 to about 1000 cells, from about 1000 to about 2500 cells, from about 2500 to about 5000 cells, from about 5000 to about 10,000 cells, from about 500 cells to about 10,000 cells, or from about 10,000 to about 20,000 cells).

The cells can be derived from a biopsy (e.g., a tumor biopsy). In some cases, the cells can be derived from one or more core biopsies (e.g., one or more biopsies having about a 14-gauge core to about a 20-gauge core biopsy). For example, the cells can be derived from one or more 18-gauge core biopsies, or from one or more 16-gauge core biopsies.

The cells can be derived from a tumor biopsy. The tumor can be associated with any type of cancer, including, without limitation, rectal cancer, lung cancer, breast cancer, colorectal cancer (CRC), kidney cancer, ovarian cancer, or any combination thereof. The cells can be derived from a single patient, or from more than one patient. In some cases, the cells can include CRC PDX cells.

When the MOS are prepared by mixing the cells with an immiscible material (e.g., oil) and a polymer, the mixing can form a plurality of nascent MOS that subsequently form the MOS. The nascent MOS can include a seeding density of about 20 to about 100 cells per droplet (e.g. about 20 to about 50 cells per droplet, about 30 to about 70 cells per droplet, about 40 to about 60 cells per droplet, or about 50 to about 100 cells per droplet). In some cases, the nascent MOS can have a seeding density configured to generate tumorspheres in the MOS of a desired quantity, a desired size, or both. Thus, in some cases, the MOS can include tumorspheres, or can include tumorsphere-like structures (e.g., in the presence of tumor-resident immune cells). The number and size of tumorspheres can be correlated with the seeding density.

In some cases, the method for generating MOS also can include determining a number of MOS (NMOS) by dividing the number of viable cells by the number of cells per droplet. The MOS generated according to the methods described herein can each contain at least 10 tumor cells (e.g., at least 20 tumor cells, or at least 30 tumor cells). In some cases, each MOS can contain from about 10 tumor cells to about 50 tumor cells.

In some cases, this document provides methods for imaging MOS. For example, images of MOS (e.g., MOS in bulk MATRIGEL® or MOS cultured in any suitable medium) can be obtained using a microscope (e.g., a bright field microscope, a confocal microscope, or a fluorescent microscope), or using any other suitable technique (e.g., liquid lens, holography, sonar, bright and/or dark field imaging, laser imaging, planar laser sheet, or high-throughput methods that include image-based analysis). In some cases, MOS surface area can be determined using any appropriate software (e.g., ImageJ software; imagej.nih.gov/ij).

In addition, in some cases the methods provided herein can include treating the MOS with one or more therapeutic agents. Such treatment, followed by an assessment of whether the therapeutic agent(s) affect the viability of the MOS, can indicate whether the therapeutic agent(s) are likely to be effective for treating a tumor in the subject from which the MOS were prepared. The one or more therapeutic agents can include, for example, a small molecule or an antibody. The one or more therapeutic agents can be applied to the MOS at any suitable concentration (e.g., from about 1 μM to about 10 μM). Moreover, the one or more therapeutic agents can include any appropriate agents. One or more of the therapeutic agents can be a chemotherapeutic agent. Non-limiting examples of therapeutic agents that can be used in the methods provided herein include oxaliplatin, irinotecan, or a combination thereof. The treating can occur less than 11 days after a biopsy acquisition (e.g., less than 5 days after a biopsy acquisition, or less than 3 days after a biopsy acquisition).

As described herein, MOS can encapsulate various cell types (e.g., tumor cells, stromal cells, and immune cells) that are resident in the tissues (e.g., tumor tissues) from which they are derived. In addition, the MOS also largely capture the genomic profiles of the tissues from which they are derived. Thus, without being bound by a particular mechanism, MOS can function as a predictive model of a patient's sensitivity to one or more drug therapies for treating a disease. For example, MOS can function as a predictive model of a patient's sensitivity to one or more chemotherapies. In some cases, MOS can function as a predictive model of a patient's sensitivity to one or more chemotherapies within 14 days of MOS preparation.

In some cases, MOS can contain an amount of fibroblasts that is less than the amount of fibroblasts found in comparative bulk organoid cultures. For example, the amount of fibroblasts encapsulated in MOS can be less than the amount of fibroblasts found in comparative bulk organoid cultures after 2 days of culturing, less than the amount of fibroblasts found in comparative bulk organoid cultures after 5 days of culturing, or less than the amount of fibroblasts found in comparative bulk organoid cultures after 7 days of culturing. The MOS also can contain functional immune cells. For example, the MOS can contain immune cells that are responsive to an immune therapy. In some cases, the MOS can contain natural killer cell markers (e.g., CD4+, CD8+, CD56+, or a combination thereof).

This document also provides methods for predicting a patient's response to a therapeutic treatment. As described herein, immune cells resident in a tissue sample (e.g., immune cells resident in a tumor tissue sample) can be encapsulated in MOS derived from the tissue sample. Because the MOS can capture the immune microenvironment of a tumor, effects of drugs that influence immune cells and/or influence the interplay between immune cells and cancer cells (e.g., checkpoint inhibitors) can be evaluated in MOS. As demonstrated herein, encapsulated immune cells in MOS can be viable and responsive to immune stimulation, such that immune therapies can be tested on resident immune cells encapsulated in MOS. In some cases, the methods provided herein can include co-culturing MOS with one or more agents associated with an immune therapy, and assaying the MOS to determine potency of the immune therapy.

Any appropriate immune therapy can be tested with a population MOS preparation. For example, an immune therapy can be an immune-oncology (IO) therapy, a checkpoint inhibitor, a T cell activator, tumor infiltrating lymphocytes (TILs), an IO therapy molecule, a MAPK inhibitor, or a combination thereof. In some cases, an immune checkpoint inhibitor can be used, such as an anti-PD1 therapy (e.g., nivolumab, pembrolizumab, cemiplimab, atezolizumab, dostarlimab, durvalumab, or avelumab) or another checkpoint inhibitor (e.g., a T-cell targeted immunomodulator, ipilimumab, TSR-022, MGB453, BMS-986016, or LAG525). In some cases, an IO therapy molecule can be used, where the IO therapy molecule includes a PD-1 blockade, TCB, or both. The immune therapy can target a human leukocyte antigen (HLA), antigens associated with the HLA, or both. The agent can include comprises a T-cell receptor-mimic antibody (e.g., ESK1, DP47, or both). In some cases, the immune therapy can be a MAPK inhibitor (e.g., vemurafenib, dabrafenib, PLX8349, cobimetinib, trametinib, selumetinib, or BVD-523). Other immune therapies that can be used include, without limitation, immunomodulators (e.g., anti-CD47 antibodies and antibody-dependent cell-mediated cytotoxicity (ADCC) therapies), apoptosis inhibitors (e.g., ABT-737, WEHI-539, ABT-199), agents targeting components of potential contributing pathways (e.g., afuresetib, idasanutlin, and infliximab), chemotherapy agents (e.g., cytarabine), cell therapies, cancer vaccines, oncolytic viruses, and bi-specific antibodies. The agent can be present in an amount of about 0.1 μg/mL to about 10 μg/mL, about 0.5 μg/mL to about 5 μg/mL, or about 1 μg/mL to about 3 μg/mL. The method can include determining an amount of cell apoptosis that occurs in tumorspheres present within the MOS following initiation of the immune therapy. The MOS can function as a predictive model for at least 12 months, at least 6 months, or at least 3 months.

In some cases, the methods provided herein can include infecting MOS with one or more viruses. For example, a virus can be used to deliver a therapeutic agent (e.g., an immune therapy) to MOS. Examples of viruses that can be used to infect MOS include, without limitation, lentiviruses, adeno-associated viruses, and influenza viruses. In some cases, a virus containing nucleic acid encoding a polypeptide (e.g., a marker, a therapeutic polypeptide, or a DNA editing polypeptide such as CRISPR-associated (Cas) nuclease), can be used to infect MOS.

In another aspect, this document features methods for treating mammals (e.g., humans, such as human patients). The methods can include, for example, predicting a patient's response to a therapeutic treatment using a method provided herein, and selecting a therapy based on the patient's predicted response. In some case, a method can include co-culturing MOS with effector immune cells, and then assaying the MOS to determine the potency of the therapy with the effector immune cells. The immune cells can be, for example, chimeric antigen receptor (CAR) T cells, tumor infiltrating lymphocytes (TILs), peripheral blood mononuclear cells (PBMCs), T cells isolated from PBMCs, T cells isolated and expanded from tumor cells, or any combination thereof.

This document also provides a MOS composition, where compositions contains a plurality of MOS, with each MicroOrganoSphere including a base material and at least one tumorsphere that includes an aggregation of cells. The plurality of MOS can include a predetermined number of cells per droplet, a predetermined number of droplets in the composition, and/or a predetermined droplet size. In some cases, the composition also can contain one or more therapeutic agents (e.g., one or more drug therapies to which the tumorsphere is responsive).

As described herein, the MOS and the original tumor from which the MOS were generated can have similar genomic profiles. In addition, the whole exome sequence of the MOS can be correlated with that of the original tumor. In some cases, the MOS and the original tumor can have similar expression patterns of immunosuppressive markers.

### Exemplary Embodiments

Embodiment 1 is a method comprising obtaining a plurality of cells derived from tissue; mixing the plurality of cells with a fluid comprising a polymer, thereby obtaining a mixture; intersecting a stream of the mixture with an immiscible material (e.g., an oil) to generate MicroOrganoSpheres (MOS).

Embodiment 2 is the method of embodiment 1, comprising demulsifying the generated MOS.

Embodiment 3 is the method of any one of the preceding embodiments, comprising culturing the generated MOS.

Embodiment 4 is the method of any one of the preceding embodiments, comprising culturing the generated MOS as suspension droplets.

Embodiment 5 is the method of any one of the preceding embodiments, wherein the polymer is a polymer matrix.

Embodiment 6 is the method of embodiment 5, wherein the polymer matrix is derived from an extracellular matrix.

Embodiment 7 is the method of any one of the preceding embodiments, wherein the MOS have an average diameter of about 250 μm to about 450 μm.

Embodiment 8 is the method of any one of the preceding embodiments, wherein the MOS have an average diameter configured to provide a three-dimensional cellular environment.

Embodiment 9 is the method of any one of the preceding embodiments, wherein the plurality of cells includes no more than 15,000 cells.

Embodiment 10 is the method of any one of the preceding embodiments, wherein the plurality of cells includes no more than 10,000 cells.

Embodiment 11 is the method of any one of the preceding embodiments, wherein the plurality of cells includes no more than 5,000 cells.

Embodiment 12 is the method of any one of the preceding embodiments, wherein the plurality of cells includes no more than 1,000 cells.

Embodiment 13 is the method of any one of the preceding embodiments, wherein the plurality of cells comprises from about 100 cells to about 20,000 cells.

Embodiment 14 is the method of any one of the preceding embodiments, wherein the plurality of cells comprises from about 500 cells to about 10,000 cells.

Embodiment 15 is the method of any one of the preceding embodiments, wherein the cells are derived from a biopsy.

Embodiment 16 is the method of any one of the preceding embodiments, wherein the cells are derived from a tumor biopsy.

Embodiment 17 is the method of any one of the preceding embodiments, wherein the cells are derived from one or more core biopsies comprising from about a 14-gauge core to about a 20-gauge core biopsy.

Embodiment 18 is the method of any one of the preceding embodiments, wherein the cells are derived from one or more 18-gauge core biopsies.

Embodiment 19 is the method of any one of the preceding embodiments, wherein the cells are derived from a tumor biopsy for one or more cancers.

Embodiment 20 is the method of embodiment 19, wherein the one or more cancers comprises rectal cancer, lung cancer, breast cancer, colorectal cancer (CRC), kidney cancer, ovarian cancer, or combinations thereof.

Embodiment 21 is the method of any one of the preceding embodiments, wherein the cells are derived from one or more patients.

Embodiment 22 is the method of any one of the preceding embodiments, wherein the cells comprise CRC patient-derived xenograft (PDX) cells.

Embodiment 23 is the method of any one of the preceding embodiments, wherein the MOS comprise tumorspheres.

Embodiment 24 is the method of any one of the preceding embodiments, wherein the MOS comprises tumorsphere-like structures in presence of tumor-resident immune cells.

Embodiment 25 is the method of any one of the preceding embodiments, wherein the mixing forms a plurality of nascent MOS that subsequently form the MOS.

Embodiment 26 is the method of any one of the preceding embodiments, wherein the nascent MOS includes a seeding density of about 20 to about 100 cells per droplet.

Embodiment 27 is the method of any one of the preceding embodiments, wherein the nascent MOS includes a seeding density of about 20 to about 50 cells per droplet.

Embodiment 28 is the method of any one of the preceding embodiments, wherein the nascent MOS includes a seeding density of about 30 to about 70 cells per droplet.

Embodiment 29 is the method of any one of the preceding embodiments, wherein the nascent MOS includes a seeding density of about 40 to about 60 cells per droplet.

Embodiment 30 is the method of any one of the preceding embodiments, wherein the nascent MOS includes a seeding density of about 50 to about 100 cells per droplet.

Embodiment 31 is the method of any one of the preceding embodiments, wherein the nascent MOS includes a seeding density configured to generate tumorspheres in the MOS of a desired quantity, size, or both.

Embodiment 32 is the method of any one of the preceding embodiments, further comprising determining a number of MOS (NMOS) by dividing a number of viable cells by a number of cells per droplet.

Embodiment 33 is the method of any one of the preceding embodiments, further comprising treating the MOS with one or more therapeutic agents.

Embodiment 34 is the method of embodiment 33, wherein the one or more therapeutic agents comprises a small molecule or an antibody.

Embodiment 35 is the method of any one of the preceding embodiments, wherein the treating comprises delivering one or more therapeutic agents at a concentration from about 1 μM to about 10 μM.

Embodiment 36 is the method of any one of embodiments 32-34, wherein the one or more therapeutic agents comprises oxaliplatin, irinotecan, or a combination thereof.

Embodiment 37 is the method of any one of the preceding embodiments, wherein the treating occurs less than 11 days after a biopsy acquisition.

Embodiment 38 is the method of any one of the preceding embodiments, wherein the treating occurs less than 5 days after a biopsy acquisition.

Embodiment 39 is the method of any one of the preceding embodiments, wherein the treating occurs less than 3 days after a biopsy acquisition.

Embodiment 40 is the method of any one of the preceding embodiments, wherein each MOS comprises at least 30 tumor cells.

Embodiment 41 is the method of any one of the preceding embodiments, wherein each MOS comprises at least 20 tumor cells.

Embodiment 42 is the method of any one of the preceding embodiments, wherein each MOS comprises at least 10 tumor cells.

Embodiment 43 is the method of any one of the preceding embodiments, wherein each MOS comprises from about 10 tumor cells to about 50 tumor cells.

Embodiment 44 is the method of any one of the preceding embodiments, wherein the MOS function as a predictive model of a patient's sensitivity to one or more drug therapies for treating a disease.

Embodiment 45 is the method of any one of the preceding embodiments, wherein the MOS function as a predictive model of a patient's sensitivity to one or more chemotherapies.

Embodiment 46 is the method of any one of the preceding embodiments, wherein the MOS function as a predictive model of a patient's sensitivity to one or more chemotherapies within 14 days.

Embodiment 47 is the method of any one of the preceding embodiments, wherein the MOS comprises an amount of fibroblasts that is less than that found in comparative bulk organoid cultures.

Embodiment 48 is the method of embodiment 47, wherein the amount of fibroblasts in the MOS is less than that found in comparative bulk organoid cultures after 2 days of culturing.

Embodiment 49 is the method of embodiment 47, wherein the amount of fibroblasts in the MOS is less than that found in comparative bulk organoid cultures after 5 days of culturing.

Embodiment 50 is the method of embodiment 47, wherein the amount of fibroblasts in the MOS is less than that found in comparative bulk organoid cultures after 7 days of culturing.

Embodiment 51 is the method of any one of the preceding embodiments, wherein the MOS comprises functional immune cells.

Embodiment 52 is the method of any one of the preceding embodiments, wherein the MOS comprises immune cells that are responsive to an immune therapy.

Embodiment 53 is the method of any one of the preceding embodiments, wherein the MOS comprises natural killer cell markers.

Embodiment 54 is the method of any one of the preceding embodiments, wherein the natural killer cell markers comprise CD4+, CD8+, CD56+, and combinations thereof.

Embodiment 55 is a method of predicting a patient's response to a therapeutic treatment, the method comprising co-culturing Patient-Derived MicroOrganoSpheres (MOS) with an agent associated with an immune therapy; and assaying the MOS to determine potency of the immune therapy.

Embodiment 56 is the method of embodiment 55, wherein the immune therapy is immune-oncology (IO) therapy.

Embodiment 57 is the method of 55 or embodiment 56, wherein the agent comprises an immune checkpoint inhibitor, a T cell activator, tumor infiltrating lymphocytes (TILs), an IO therapy molecule, or a combination thereof.

Embodiment 58 is the method of embodiment 57, wherein the immune checkpoint inhibitor comprises an anti-PD1 therapy (e.g., nivolumab).

Embodiment 59 is the method of embodiment 57, wherein the IO therapy molecule comprises a PD-1 blockade, a T-cell bispecific antibody (TCB), or both.

Embodiment 60 is the method of embodiment 55, wherein the immune therapy targets a human leukocyte antigen (HLA), antigens associated with the HLA, or both.

Embodiment 61 is the method of embodiment 55, wherein the agent comprises a T-cell receptor-mimic antibody.

Embodiment 62 is the method of embodiment 55, wherein the T-cell receptor-mimic antibody comprises ESK1, DP47, or both.

Embodiment 63 is the method of any one of embodiments 55-62, wherein the agent is present in an amount of about 0.1 μg/mL to about 10 μg/mL.

Embodiment 64 is the method of any one of embodiments 55-63, wherein the agent is present in an amount of about 0.5 g/mL to about 5 μg/mL.

Embodiment 65 is the method of any one of embodiments 55-64, wherein the agent is present in an amount of about 1 μg/mL to about 3 μg/mL.

Embodiment 66 is the method of any one of embodiments 55-65, comprising determining an amount of cell apoptosis that occurred in tumorspheres present within the MOS following initiation of the immune therapy.

Embodiment 67 is the method of any one of embodiments 55-66, wherein the method provides a predictive model for at least 12 months.

Embodiment 68 is the method of any one of embodiments 55-67, wherein the method provides a predictive model for at least 6 months.

Embodiment 69 is the method of any one of embodiments 55-68, wherein the method provides a predictive model for at least 3 months.

Embodiment 70 is a method of treating a patient, the method comprising: (a) predicting a patient response to a therapeutic treatment as recited in embodiment 55; and (b) selecting a therapy based on the predicted patient response.

Embodiment 71 is a method for predicting a patient's response to a therapy, the method comprising: (a) co-culturing Patient-Derived MicroOrganoSpheres (MOS) with effector immune cells; and (b) assaying the MOS to determine potency of the therapy with the effector immune cells.

Embodiment 72 is the method of embodiment 71, wherein the effector immune cells are selected from the group consisting of chimeric antigen receptor (CAR) T cells, tumor infiltrating lymphocytes (TILs), peripheral blood mononuclear cells (PBMCs), T cells isolated from PBMCs, T cells isolated and expanded from tumor cells, and combinations thereof.

Embodiment 73 is the method of any one of embodiments 55-72, wherein the MOS is formed by the method of any one of embodiments 1-54.

Embodiment 74 is a MicroOrganoSphere composition comprising a plurality of MOS with each MOS including a base material and at least one tumorsphere, wherein the plurality of MOS comprise a predetermined number of cells per droplet, a predetermined number of droplets in the composition, and/or a predetermined droplet size.

Embodiment 75 is the composition of embodiment 74, comprising one or more drug therapies.

Embodiment 76 is the composition of embodiment 74, wherein the at least one tumorsphere is responsive to one or more drug therapies.

The invention will be further described in the following example, which does not limit the scope of the invention described in the claims.

### Example

### Patient-Derived MOS Enable Clinical Precision Oncology

### Methods and Materials

Microfluidic chip fabrication and design: Microfluidic chips were fabricated out of silicon wafers (Wafer Pro, Santa Clara, CA). Details of manufacturing microfluidic features in silicon are described elsewhere (Rius et al., “Introduction to Micro-/Nanofabrication,” In: Bhushan B. (eds) Springer Handbook of Nanotechnology. Springer Handbooks. Springer, Berlin, Heidelberg, 2017). Briefly, designs were imprinted onto a 6″ silicon wafer using standard photolithography techniques and features were etched using Deep Reactive Ion Etching (DRIE) in a clean room facility. Once cleaned, a borofloat glass cover slide (PG&O; Santa Ana, CA) was bonded to the silicon chip using anodic bonding. After bonding, the microfluidic channels were coated with Aquapel (Aquapel Glass; Cranberry Twp, PA) to create a hydrophobic surface. Following coating, channels were rinsed with 3 mL of Novec 7500 engineered fluid (3M; Saint Paul, MN) and then baked at 60° C. for 20 minutes.

MOS generator assembly: MOS generation took place inside a 1.7 cu. ft. miniature refrigerator to keep the temperature-sensitive gel from polymerizing during generation. Fluigent FlowEZ (Fluigent; La Kremlin-Bicetre, France) pressure sources were attached to the top of the refrigerator. Air tubing was connected to the reagent and sample reservoir PCaps (Fluigent) through the top of the refrigerator via two drilled holes. Pumps were operated manually according to the manufacturer's recommendations. Chips were assembled inside a custom fabricated manifold that contained ports to connect the reagent and sample reservoirs to the chip. All components were placed inside the refrigerator. The door was kept closed when processing temperature sensitive material. MOS generation was imaged by assembling the camera and lens components listed in Table 3 and placing the camera directly over the chip.

Patient specimens: Tissue sections (about 1-2 cm³) of metastatic colorectal cancer, lung cancer, ovarian cancer, kidney cancer, breast cancer, and non-tumor tissue were obtained from surgically resected specimens provided by Duke BioRepository & Precision Pathology Center (BRPC) with patient consent. The entire experimental protocol was conducted in compliance with institutional guidelines. Samples were confirmed as tumor or normal tissue via histopathological assessment. IRB Approvals (IRB #Pro00089222) and Research protocols were approved by the relevant institutional IRBs.

Tumor tissue processing and MOS generation: All tumor and non-tumor tissues were kept in transfer media and on ice after dissection. Ten percent of the tissue sample was frozen down in OCT immediately, and the remainder was minced before mixing with 10 mL of enzymatic solution. The enzymatic solution consisted of a collagenase-based digestion solution containing CaCl₂ (3 mM), Collagenase (1 mg/mL) (Sigma Cat #11088858001), DNase I (0.1 mg/mL) (STEMCell technology Cat #07900), Y-27632 (10 μM) (STEMCell technology Cat #72302), and Primocin (100 μg/mL) (Fisher Scientific Cat #NC9141851). Minced tissue samples were dissociated with gentle agitation in enzymatic solution for 30 minutes at 37° C. before a first cell quality check. If large cell clumps were observed, an additional 15-20 minutes of digestion was performed until the tissue was mostly dissociated into single cells. After digestion, cells were filtered through a 70 μM cell strainer, and yield and cell viability were determined by a Countess II cell counter using a previously described Trypan blue method. The initial cell number inserted into MOS was dependent on the intended application. For example, a single tumor cell per MOS was used for clonal diversity studies, while 20 tumor cells per MOS was typically used for testing chemotherapy, as that number provided the best tradeoff between tumorsphere establishment speed and the number of MOS for testing different conditions. Thirty (30) to 50 tumor cells (and proportional numbers of immune cells from the same digested sample) per MOS were more appropriate for IO assays. The same cell density was seeded using traditional MATRIGEL® methods as a comparison. Demulsified MOS were layered with tumor medium and seeded in 6-well low binding plates. Cells in MATRIGEL® were loaded in 24-well plates and grown in the tumor medium. Medium was changed every 3 days.

H&E staining of original tumors and MOS: Tissues and MOS were processed for paraffin sectioning. MATRIGEL®-embedded MOS were collected after centrifuging in a 15 mL tube at 100 g for 3 minutes. The supernatant was removed, and MOS were fixed in 2% paraformaldehyde (PFA) with 0.1% glutaraldehyde for 30 minutes at room temperature before washing in 1×PBS and embedding in Histogel. Fresh cancer tissue was embedded in paraffin after formalin fixation. After deparaffinization, 5-μm sections were stained with hematoxylin-eosin (H&E). MOS and primary tumor sections were evaluated for morphological characterization by a pathologist.

MOS and organoid imaging: Images of MOS and organoids in bulk MATRIGEL® were acquired using a Leica microscope (Leica, USA) at day 1, day 3, day 5, and day 7 after initial plating, and organoid surface area was quantified using ImageJ software (Wayne Rasband, NIHR, USA; imagej.nih.gov/ij). To calculate the average size (area) of the organoids, more than 40 tumorspheres in MOS or organoids in MATRIGEL® for each tumor sample were manually quantified, and statistical analysis was performed using Prism 8.

### Genomic and Transcriptomic Analysis on Tumor Tissue Samples and Matched MOS

DNA extraction and WES sequencing: MOS developed on day 7 were harvested for DNA extraction. DNA was extracted using a Zymo Quick-DNA Microprep kit (Zymo Research #D2030) according to the manufacturer's protocol. DNA was quantified using a NanoDrop. Tumor samples and matched tumor-derived MOS were analyzed using whole-exome sequencing (WES) by Novagene using an Illumina Novaseq 6000 sequencer.

Analysis of mutation profiles for tumors and matched MOS: A total of >0.4 μg DNA per sample was used as input. The effective sequencing depth was above 50× (6G) per sample. Prior to alignment, adaptors were trimmed from raw sequencing data using TrimGalore. The resulting fastq files were then aligned to the human reference genome (bg38) using BWA. Duplicate BAM files from matched samples were merged and filtered to remove duplicates and non-chromosomal reads. Sequence variants were then called using GATK's HaplotypeCaller pipeline (version 4.2.0). Variants were filtered based on quality by depth (QD<2.0), mapping quality (MQ<40.0), Fisher strand (FS>60.0), strand odds ratio (SOR>4.0), mapping quality rank sum (MQRankSum<−12.5), and read position rank sum (ReadPosRankSum<−8.0). Finally, variants were annotated using snpEff.

Disruptive variants (e.g., missense, stop-gained, disruptive inframe indels, 3/5′ UTR, splice acceptors, and splice donor variants) were selected for downstream analysis. Each unique mutation (classified as a specific position-base-alternate combination) was binarized for each sample according to presence-absence. The resulting binary vectors were used to calculate Jaccard similarity scores and generate Venn diagrams and a presence-absence table. Genes represented in the presence-absence table (not shown) were limited to the 25 most commonly mutated genes by cancer type according to The Cancer Genome Atlas (TCGA).

Drop-seq gene expression library preparation and data analysis: Frozen PBMCs were thawed, and count and cell viability were measured by Countess II. For single cell RNA-seq, 200K cells were aliquoted, spun down, resuspended in 30 μl PBS+0.04% BSA+0.2 U/μl RNase inhibitor, and counted using Countess II. The scRNA Drop-seq libraries were generated using a Dolomite Nadia machine following the manufacturer's protocol. Libraries were pooled and sequenced using Illumina NovaSeq platform with the goal of reaching saturation or 20,000 unique reads per cell on average. Sequencing data were used as inputs to the Drop-seq pipeline published by the Broad Institute (github.com/broadinstitute/Drop-seq). Gene count matrices were produced using the first 4,000 cellular barcodes with the largest number of reads associated with each index.

10× Next GEM 3′ single cell library preparation and data analysis: For single cell RNA-seq on ESK1* drug treated MOS and original tissue tumor cells, MOS were first generated from lung tumor tissue (case #805). ESK1* drug (1 μg/mL) was added into culture medium on day 5 MOS for 24 hours before cells were collected to perform scRNA seq library preparation. During library preparation, 200K cells were aliquoted, spun down, resuspended in 30 μl PBS+0.04% BSA+0.2 U/μl RNase inhibitor, and counted using Countess II. GEM generation, post GEMRT cleanup, cDNA amplification, and library construction were performed following 10X Genomics Single Cell 3′ v3.1 chemistry. Quality was assessed using Agilent DNA tape screen assay. Libraries were then pooled and sequenced using Illumina NovaSeq platform with the goal of reaching saturation or 20,000 unique reads per cell on average. Sequencing data were used as inputs to the 10× Genomics Cell Ranger pipeline to demultiplex BCL files, generate FASTQs, and generate feature counts for each library.

Dimensionality reduction and cell type annotation: Gene-barcode matrices generated using the DigitalExpression script from the Broad Drop-seq pipeline were analyzed using Seurat 3 with the default parameters unless otherwise specified. Cells with >2,500 genes detected were removed from the analysis. Counts were log-normalized, and the top 2,000 variable features were identified. Principal component analysis was performed using these variable genes, and the top 30 principal components were used for downstream analysis. UMAP dimensionality reduction was performed using the top 20 principal components identified using the Harmony package. Graph-based clustering was performed with resolution=1. Cell types were inferred by using the HumanPrimaryCellAtlasData(rdrr.io/github/LTLA/celldex/man/HumanPrimaryCellA tlasData.html) function from the SingleR package. Labels were confirmed by identification of differentially expressed genes using the FindAllMarkers function from Seurat (www.rdocumentation.org/packages/Seurat/versions/4.1.0/topics/FindAllMarkers) and visualization of marker genes plotted as kernel density on UMAPs using the Nebulosa package. To perform differential expression analysis, cell type labels were grouped into four groups: tumor cells, fibroblasts, lymphoid cells, and myeloid cells.

Pseudo-bulk Differential Expression Analysis: Three biological replicates from patients with lung cancer were used for pseudo-bulk differential expression analysis. Specifically, datasets generated from primary tissue were compared with datasets generated from MOS to determine changes in gene expression between the two platforms. Gene count values from cells with the same cell type label were aggregated into a single matrix. The model design formula included a term indicating which samples were produced from primary tissue or MOS. Significance testing was performed using the glmQLFit function from the EdgeR package (www.rdocumentation.org/packages/edgeR/versions/3.14.0/topics/glmQLFit), and false discovery rate adjustment was performed for the p-values. Genes with an absolute log-fold change >1 and adjusted p-value <0.05 were considered significantly differentially expressed between the two conditions. Intersections in the differentially expressed gene list for each cell type were visualized as an UpSet plot. using the UpSetR package. A volcano plot was generated for the pseudo-bulk results from each of the cell types using the Enhanced Volcano package and the same significance thresholds.

The remaining two samples were collected from patients with either kidney cancer (n=1) or ovarian cancer (n=1). For these samples, log-fold change in gene expression was compared for libraries produced from primary tissue or MOS, and no p-values were reported. Gene counts were averaged for each cell type using the AverageExpression function from Seurat (www.rdocumentation.org/packages/Seurat/versions/4.1.0/topics/AverageExpression), and log-transformed values were plotted to compare the samples produced from primary tissue and MOS. Genes with a log-fold change>1 were labeled in red, and genes with average log expression>1.5 were labeled in black to provide context for highly expressed genes in each cell type.

Identification of Conserved Gene Expression: The FindConservedMarkers (www.rdocumentation.org/packages/Seurat/versions/4.1.0/topics/FindConservedMark ers) function from Seurat was applied to cells of each cell type, and genes with conserved expression and log-fold change enrichment>0.5 were identified. The top five markers with the highest log-fold change enrichment for each cell type were visualized using the DotPlot (satijalab.org/seurat/reference/dotplot) function from Seurat. Important cancer gene expression markers CD274 (PD-L1), PDCD1 (PD-1), and TGFB1 (TGF-beta) were also specifically visualized to compare expression in the tumor cells, lymphoid cells, and fibroblasts, respectively. Expression of these markers was plotted as UMAPs using the Nebulosa package and labeled on the respective volcano plots.

Flow cytometry analysis: MOS and bulk MATRIGEL® established by day 7-9 were dissociated into single cells using TrypLE treatment and incubated in 37° C. for 5 minutes. Dissociated cells were washed with PBS+0.04% BSA and stained with either anti-human Vimentin antibody (CST Cat #, 1:100) combined with PE or anti-human EpCAM (Biolegend Cat #324205, 1:250), at room temperature for 20 minutes. Cells were washed once again with PBS+0.04% BSA before staining with goat anti-mouse Alexa Fluro 488 secondary antibody (Invitrogen Cat #A32723) for 15 minutes at room temperature. Cells were washed once more with PBS+0.04% BSA before flow assay. Sytox blue dead cell stain (A34857) was added as 1:1000 dilution to gate out dead cells in the assays. All flow assays were performed using a Sony SH800 FACS sorter, and flow data were analyzed using FlowJo.

Drug High-Throughput Screening: Automated liquid handling was provided by the Echo Acoustic Dispenser (Labcyte) for drug administration or Well mate (Thermo Fisher) for cell plating, and assays were performed using a Clariosean plate reader (BMG Labtech). Immediately prior to cell plating, 384 well plates were stamped with 119 FDA-approved drug compounds at a final concentration of 1 μM. The compound library (Approved Oncology Set VI) was provided by the NCI Developmental Therapeutics Program (https://dtp.cancer.gov/). MOS were plated in these drug pre-coated plates at 100 MOS/well with each MOS containing 30 cells/droplet. Cell viabilities were assessed via CellTiter-Glo Luminescent Cell Viability Assay (Promega, USA) 72 hours after cell plating. Percent cytotoxicity was quantified using the following formula: 100*[1-(average CellTiterGlo^{drug}/average CellTiterGlo^{control})].

HLAA2 plasmid, lentivirus packaging, and MOS infection: The HLA-A2 (insert) was amplified from cDNA library prepared with RNA from NCI-H1755 (ATTC, CRL-5892) using sense primer GGTCGCCACCATGGCCGTCATGGCTCCCCG (SEQ ID NO:1) and antisense primer: GGCCGCTTTACACTTTACAAGCTGTGAGAG (SEQ ID NO:2). The linearized plasmid (recipient) was amplified from pLenti CMV GFP Puro plasmid (Addgene: 17748) using sense primer TTGTAAAGTGTAAAGCGGCCGCGTCGACAA (SEQ ID NO:3) and antisense primer TGACGGCCATGGTGGCGACCGGTGGATCCT (SEQ ID NO:4). The PCR products (both insert and vector) were purified using Gel DNA Recovery Kits (Zymo, D4007). The insert was then cloned into the vector by Gibson assembly (NEB, E2611S). Lentiviral particles were produced by co-transfection of HEK 293T cells using Lipofectamine 2000 transfection. Briefly, HEK293T cells were co-transfected with 10 μg of transgene plasmid, 10 μg of packaging plasmid pCMVR8.74 (Addgene: 22036) and 5 μg envelope plasmid pMD2.G (Addgene: 12259). After 12 hours, the transfection medium was changed. Recombinant lentiviruses were harvested at 24 and 48 hours. The supernatant containing the viral particles was then concentrated using the Lenti-X Concentrator kit (Takara, 631232). Concentrated lentiviral particles were then aliquoted and stored at −80° C. until use.

MOS infection with HLA-A2 expressing lentivirus: HLA-A2- and DsRed-expressing lentivirus was added into lung tumor MOS culture (MOI=5-6) when MOS was established. After 3 days of incubation, infection efficiency was evaluated by observing DsRed expression under the microscope. HLA-A2 gene expression and HLA-A2 antigen expression were evaluated using flow cytometry.

RNA extraction and qRT-PCR: To quantify HLA-A2 gene expression in lung tumor samples, RNA was extracted using a Norgen single cell RNA purification kit (Norgen Biotek Cat #51800). cDNA reverse transcription was performed using SuperScript IV Vilo MasterMix with ezDNase (Thermo Fisher Cat #11756050). HLA-A2 gene was amplified using forward primer TGAAGGCCCACTCACAGACTC (SEQ ID NO:5) and reverse primer: CCCACGTCGCAGCCATACATC (SEQ ID NO:6).

### Immuno-Oncology Potency Assay

Human peripheral blood mononuclear cell (PBMC) and patient TIL expansion: Human PBMC was purchased from STEMCell technology (Cat #70025.1). Tumor TILs were generated from dissociated tumor tissue cells. Dissociated cells (0.5×10⁶) were collected for the purpose of TIL expansion. Cells were resuspended in IMMUNOCULT™-XF T Cell Expansion Medium supplemented with 6000 IU/mL Recombinant Human IL-2 (Miltenyi Biotec Cat #130-097-743). TILs were maintained for 1 week before splitting and the medium was changed to one with CD3/CD28/CD2 T cell activator (STEMCell technology, Cat #10971) for further expansion.

ESK1* drug preparation: ESK1* TCB and Negative TCB (DP47) were supplied by Roche. Drugs were aliquoted immediately after receiving to avoid multiple freeze-thaw. Drugs were used at 1 μg/mL or 10 μg/mL in all potency assays.

IO assay and Incucyte live cell imaging: MOS generated from primary tumor tissue were plated into 96-well plates with a density of 30-50 MOS per well supplied with culture medium without Y compound. Day 3 or day 4 MOS were treated with ESK1*, DP47 or Nivolumab for at least 3 days and imaged in Incucyte during the treatment. When performing immune cell potency assay, pre-activated PBMCs or matched TILs were stained with Cytolight Rapid red dye following manufacturer instructions. Briefly, Cytolight Rapid Red dye in one vial was diluted with 20 μl DMSO and further diluted 10-fold in PBS. PBMC or TILs were incubated at 37° C. with 5 μl diluted Cytolight Red dye (500X) in PBS for 25 minutes. After one wash with PBS, PBMC or TILs were counted and resuspended into wells containing MOS and culture medium at an effector: target ratio of 5:1 or 10:1. Annexin V green dye, Caspase 3/7 green dye, or Cytotox green dye was added into each well following manufacturer instruction. Plates were loaded into Incucyte S3 and images were taken every 2 hours for 4-5 days.

IO assay with immunotherapy and MHC block: Lung tumor MOS were incubated with anti-MHC I/II antibodies (W6/32; Tu39, Cat #361702, Biolegend) at a concentration of 20 μg/mL for 45 minutes at 37° C. before seeding into a 96-well plate at a density of 30-50 MOS per well supplied with lung tumor culture medium without Y-27632. Non-MHC-blocked MOS were used as controls. Matched TILs were added to each well at a 5:1 effector: target ratio. Nivolumab was added to wells at a working concentration of 10 μg/mL. The CD2/CD3/CD28 T cell activator reagent was added at a working concentration of 25 μl/mL. Annexin V was added into each well following the manufacturer's instructions.

Incucyte imaging data analysis: Raw images from phase wand green and red fluorescence channels were exported, and MOS were manually drawn using the “Labelme” image annotation software. The fluorescent images and labels were then fed into a Python script that binarized the images using a constant threshold, counting all pixels in the red image above the threshold as “red,” all pixels in the green image above the threshold as “green,” and all pixels that were above the threshold in both the red and green images as “yellow.” These pixels were then grouped according to which MOS (if any) they belonged to, and the script then exported a CSV file containing, for each well, for each time, for each MOS labeled in the associated image, the count of red, green, and yellow pixels contained within that MOS at that time.

Quantification and Statistical Analysis: T-tests were performed using Prism 8.0. p<0.05 was considered significant.

### MOS Generation and Establishment

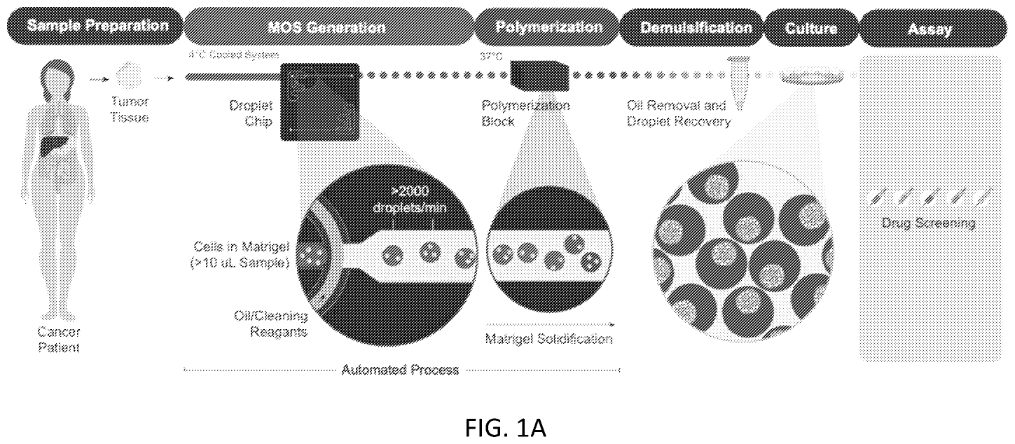

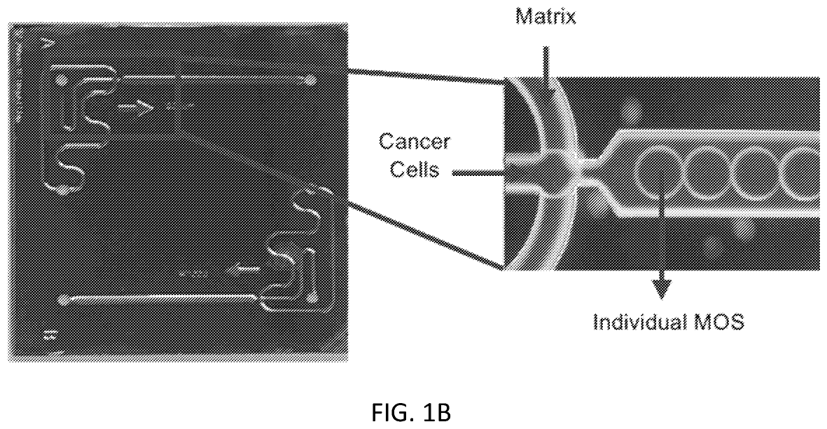

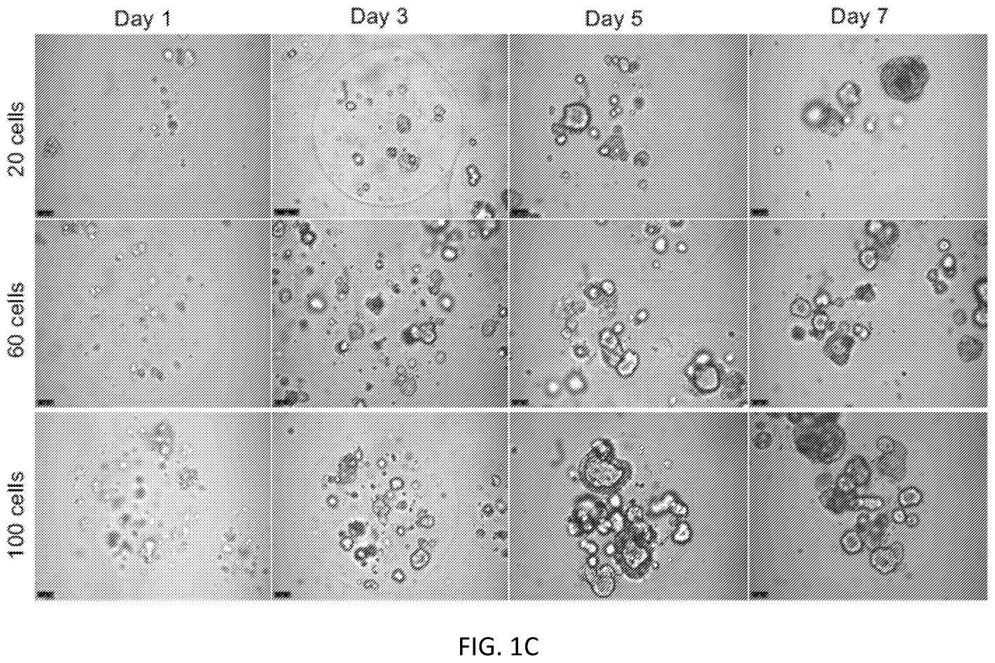

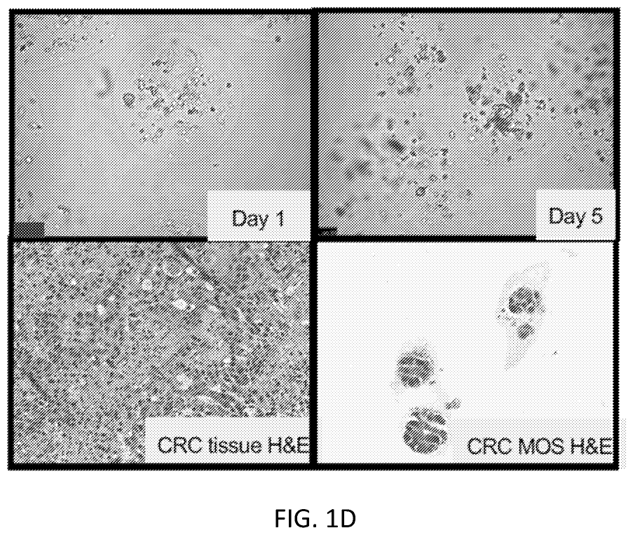

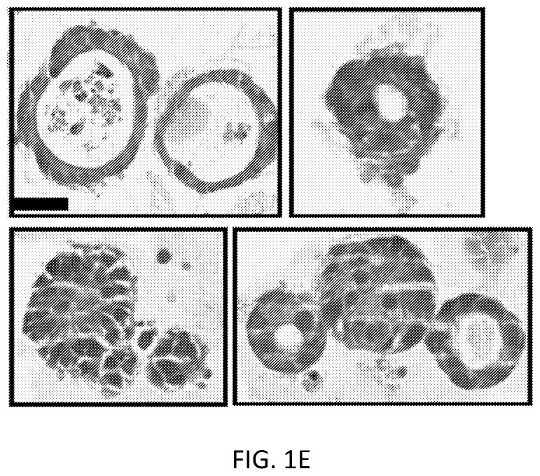

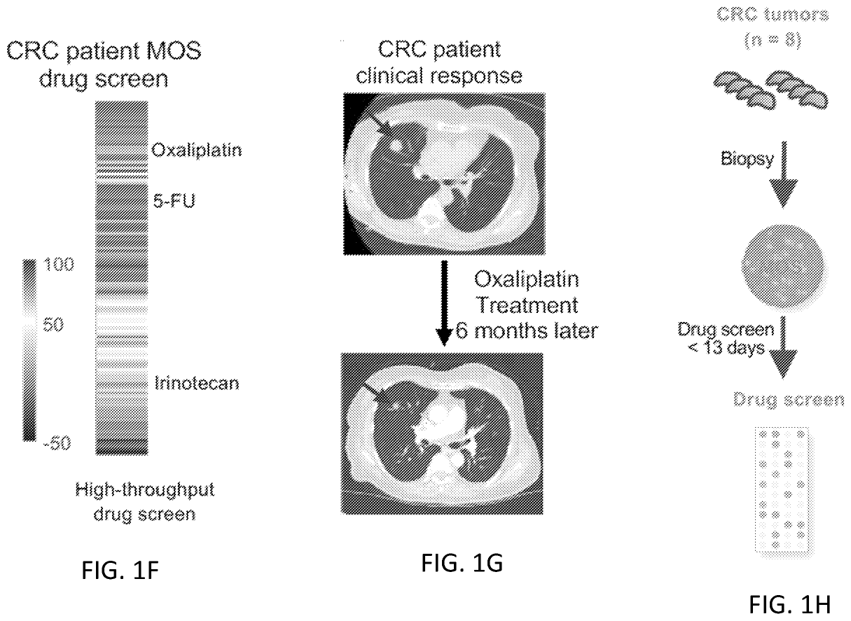

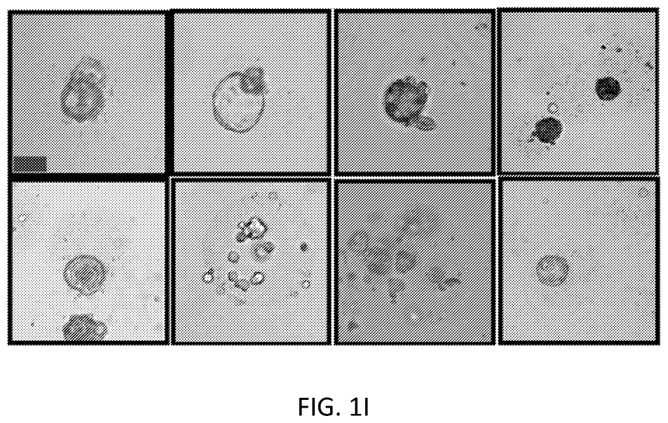

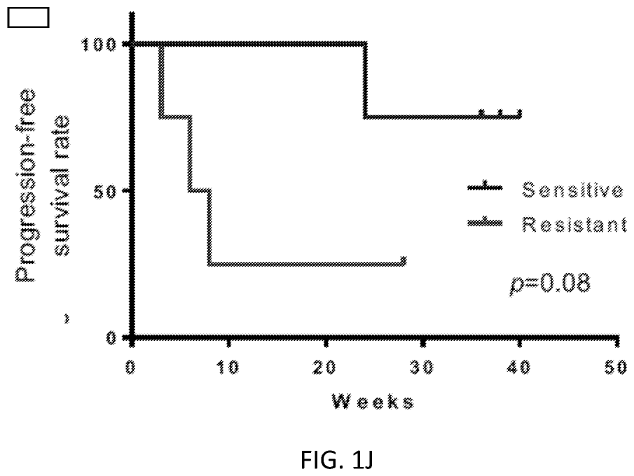

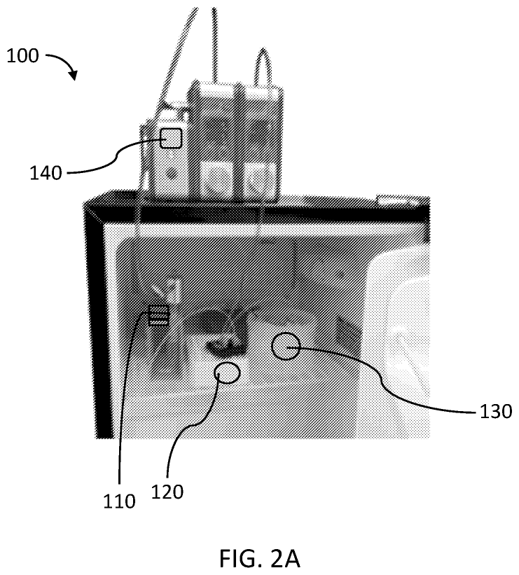

To establish a precision medicine pipeline that can be used to guide patient care, a droplet-based microfluidics technology was developed to rapidly generate patient-derived models of cancer in a reliable manner (FIG. 1A). The core principle involved adding suspended cells from primary tissue to a 3D-extracellular matrix (MATRIGEL®) followed by mixing with a biphasic liquid (oil) to generate microfluidic-based droplet MOS. The generated MOS were demulsified to remove excess oil and then cultured as suspension droplets.

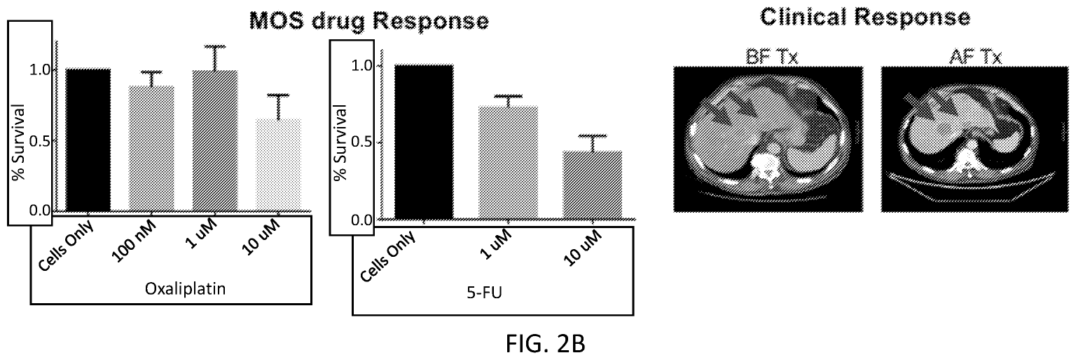

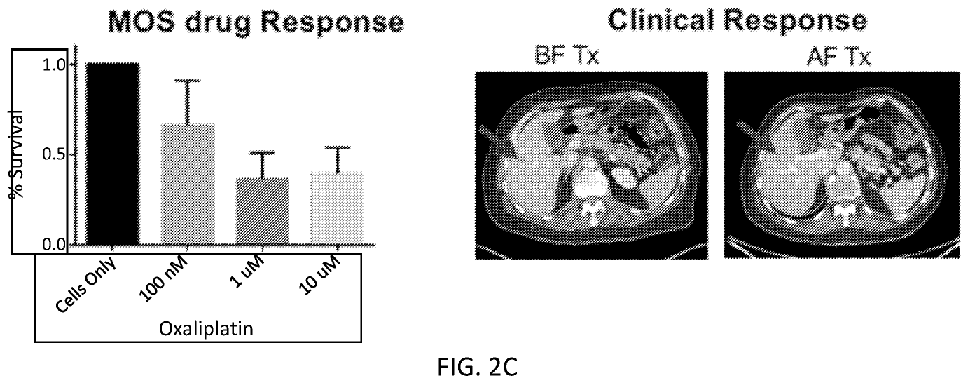

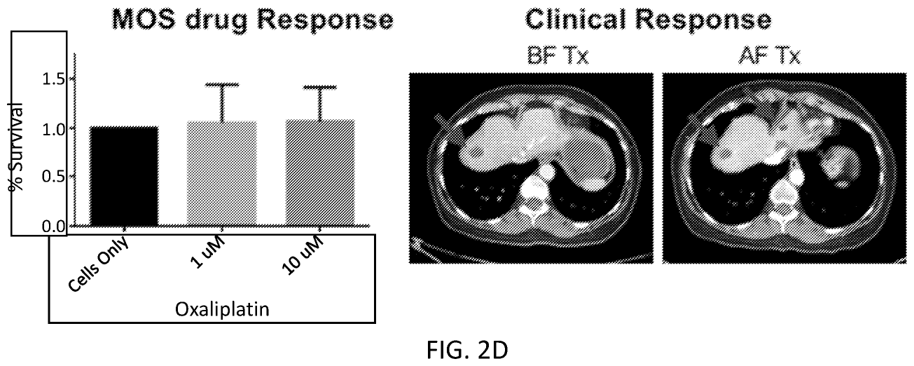

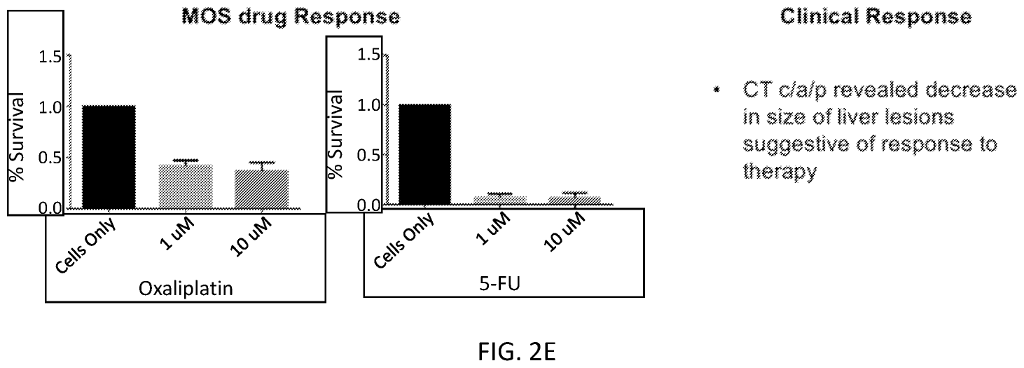

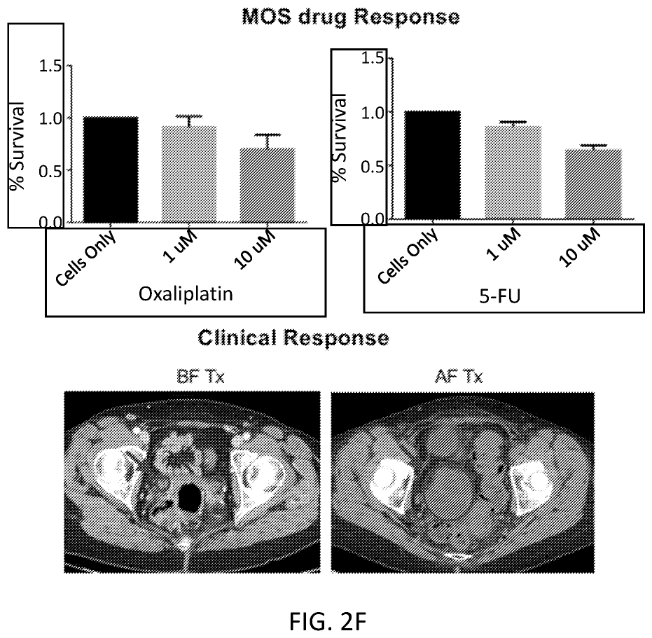

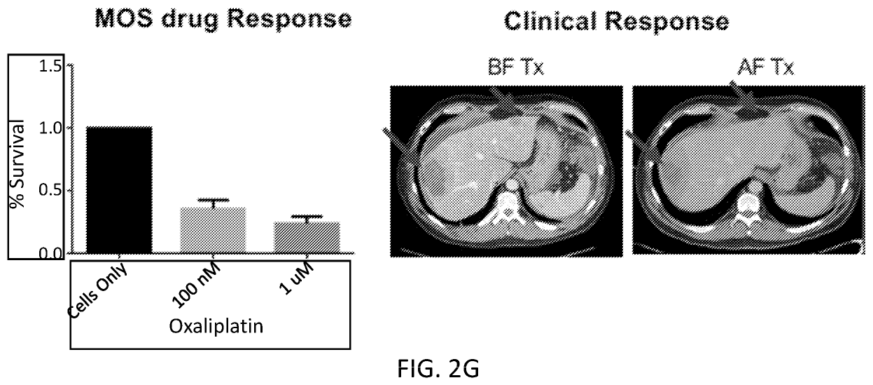

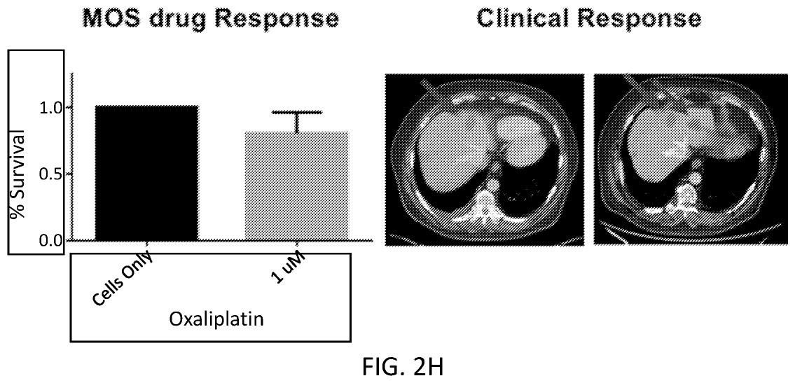

The basis of the pipeline is a benchtop machine for the generation of MOS (FIGS. 1B and 2A; TABLE 3), Important design features of the device included reservoirs for loading both the oil and sample phases directly onto a custom microfluidic chip followed by positioning of the sample outlet on the backside of the chip for direct dispensing into a MOS recovery vessel. Attached pressure sources (e.g., Fluigent FlowEZ) were used to control the flow of oil and sample fluids into the custom microfluidic chip through tubing connected via a clamped manifold. A 15 mL conical tube containing oil (110) and a 1.5 mL Eppendorf tube kept on ice containing the cell/MATRIGEL® sample mixture (120) were pressurized to drive flow through the microfluidic chip found inside the chip holder (130). The device was placed in a refrigerator with tubes connected to pumps (140) on the outside. The sample and oil met at a “T” junction (FIG. 1B) where the sample was “pinched” into droplets by the oil phase as it entered a collection channel. The system was compatible with temperature sensitive MATRIGEL®. Both the 4° C. sample and 37° C. collection blocks were integrated into the device, which allowed MATRIGEL® to flow through microfluidic channels and then quickly solidify at higher temperatures. The channel and chamber heights were engineered to generate MOS that averaged 250 μm to 450 μm in diameter, as these dimensions provided a 3D environment that was well-suited for a variety of cell numbers and sizes. The device could generate MOS from as few as 15,000 cells from 18-gauge core biopsies, a sample size typically too small for reliable generation of conventional organoids for therapeutic profiling within the clinical time constraint.

The device was first used to generate MOS from CRC PDX cells. CRC MOS growth was monitored at different seeding densities (20-100 cells per droplet), demonstrating that MOS established tumorsphere-like structures (FIG. 1C). The number and size of tumorspheres increased with the seeding density per droplet. MOS were then generated from clinical CRC biopsies (FIG. 1D) and shown to have various morphologies (FIG. 1E). The number of MOS was determined by the number of viable cells divided by the number of cells per droplet.

### MOS Predict Patient Drug Response in a Prospective Clinical Study

Since clinical treatment decisions are often made within 10-14 days of diagnosis, an ideal diagnostic assay would give results within 14 days and use minimal tissue (e.g., core biopsies) to predict clinical outcome. In an initial study, a biopsy was obtained from a patient presenting with metastatic rectal cancer, and MOS (30 tumor cells per MOS) were established within 8 days of biopsy. An in vitro high-throughput drug screen was performed by treating the MOS with the Approved Oncology Set VI panel (provided by the NCI Developmental Therapeutics Program), which contained 119 different FDA-approved small molecule inhibitors at 1 μM concentrations, and then analyzing treatment responses. The MOS were sensitive to oxaliplatin (% killing >50%) and resistant to irinotecan (% killing <50%) (FIG. 1F). The entire process was performed within 11 days of biopsy acquisition. Consistent with the MOS prediction, the patient's tumor still responded to oxaliplatin-based therapy 6 months later (FIG. 1G).

A prospective clinical study was then designed and conducted. Core biopsies (18-gauge) were obtained from seven additional patients presenting with metastatic CRC, MOS were generated, and drug testing was performed (FIGS. 1H and 1I). Patient demographic information and mutation status are shown in TABLE 1. MOS (30 tumor cells per MOS) were generated and responses to oxaliplatin were tested within 13 days (9.9 days on average) from time of biopsy for all eight biopsy samples, with a success rate of 100% (8/8) (TABLE 2). Given the limited tissue volume, dosages of 1 μM and 10 μM were selected based on studies disclosed elsewhere (Vlachogiannis et al., Science 359, 920-926, 2018, Ooft et al., Science Translational Med 11, 2019; Ganesh et al., Nat Med 25, 1607-1614, 2019; and Yao et al., Trends Immunol 41, 652-664, 2020). The same cut-off as measured via Cell Titer Glo was used. Among the eight patients, four had oxaliplatin-sensitive MOS and four had oxaliplatin-resistant MOS (FIG. 1J).

All eight patients received oxaliplatin-based therapy per usual treatment guidelines. Patient outcomes were subsequently evaluated by CT scan before and after oxaliplatin treatment (FIGS. 2B-2H), and time-on-treatment was compared to MOS oxaliplatin sensitivity. The four patients whose MOS were sensitive to oxaliplatin all responded clinically and stayed on treatment past 20 weeks (and three of four still remained on treatment close to 40 weeks), whereas three out of the four patients with resistant MOS did not respond to oxaliplatin treatment and were taken off treatment within 10 weeks (FIG. 1J). The remaining patient from the resistant MOS group on initial CT scan had a mixed response to therapy, but given the clinical benefit, the patient was continued on therapy. A subsequent CT scan showed response to therapy and the patient remained on treatment until 28 weeks, when liver resection was performed to remove the metastatic lesion (FIG. 2B and TABLE 2).

This proof of concept clinical study suggests that MOS can be reliably generated from 18-gauge biopsies of metastatic CRC tumors and then be used to test sensitivity to frontline chemotherapy within 14 days. Our initial results demonstrate that this workflow can largely predict patient outcomes, albeit larger trials are needed to further validate clinical applicability.

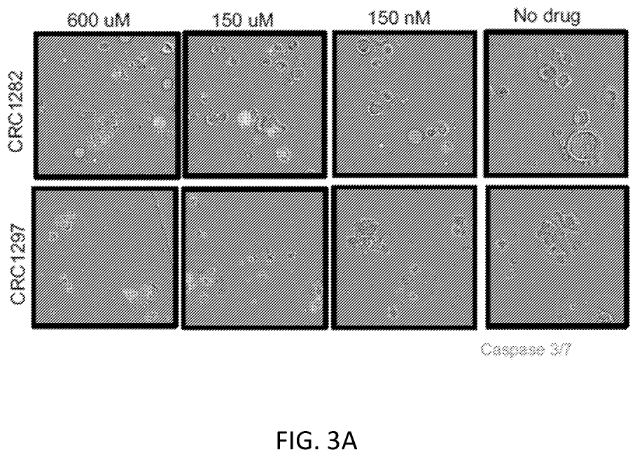

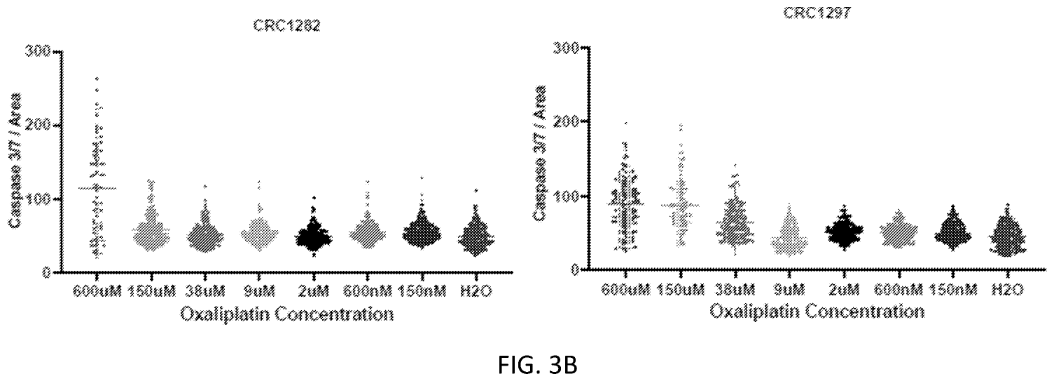

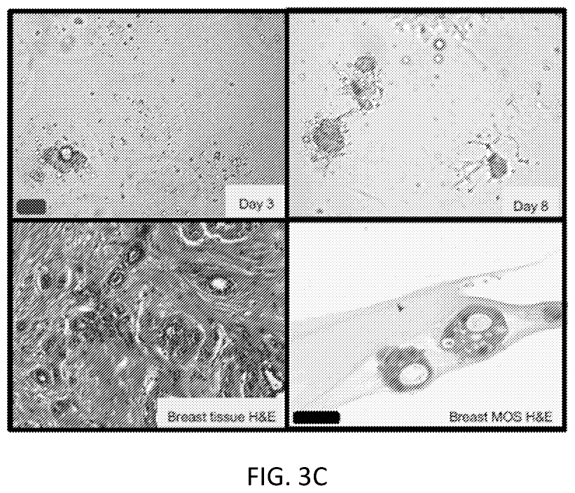

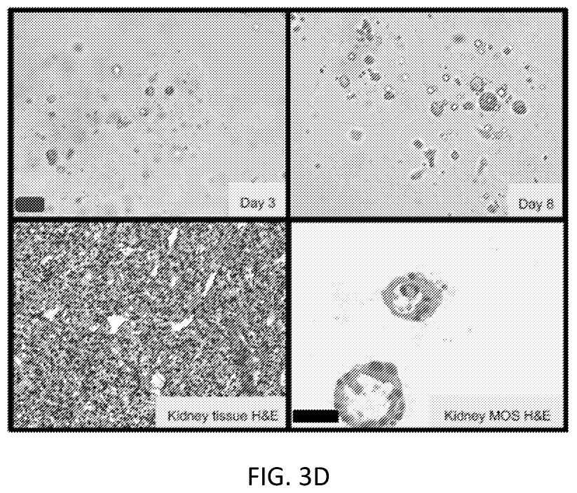

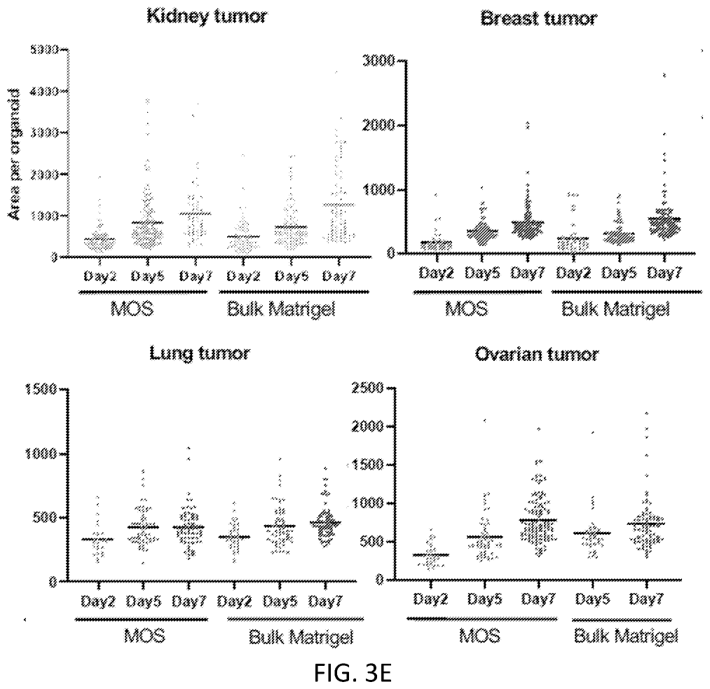

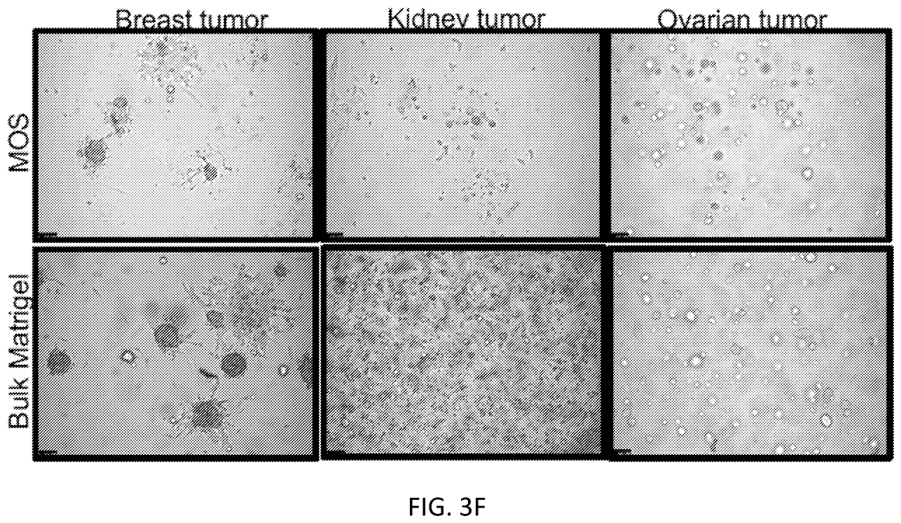

Cell death for each MOS was measured by imaging the Caspase 3/7 fluorescence signal and normalizing it by the cell surface area inside each MOS. Treatment of two available CRC MOS lines (20 cells per MOS) that were resistant to oxaliplatin showed that only the highest dosages induced significant cell killing, with heterogeneity among different MOS (FIGS. 3A and 3B).

### Tumor Stromal and Immune Cells in MOS

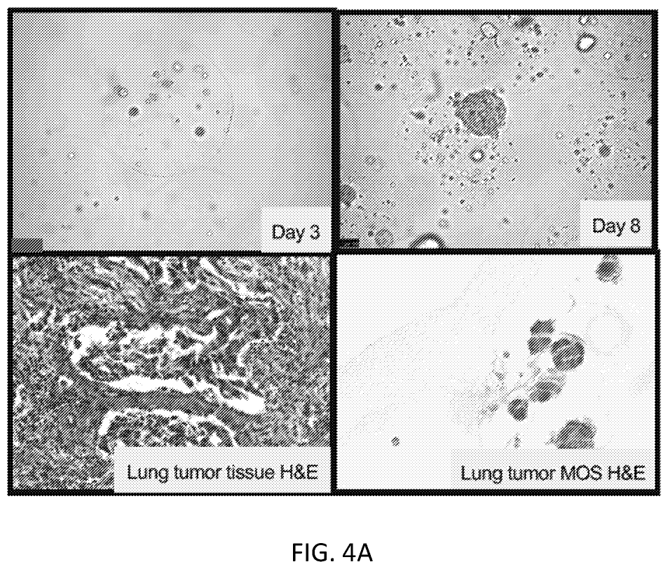

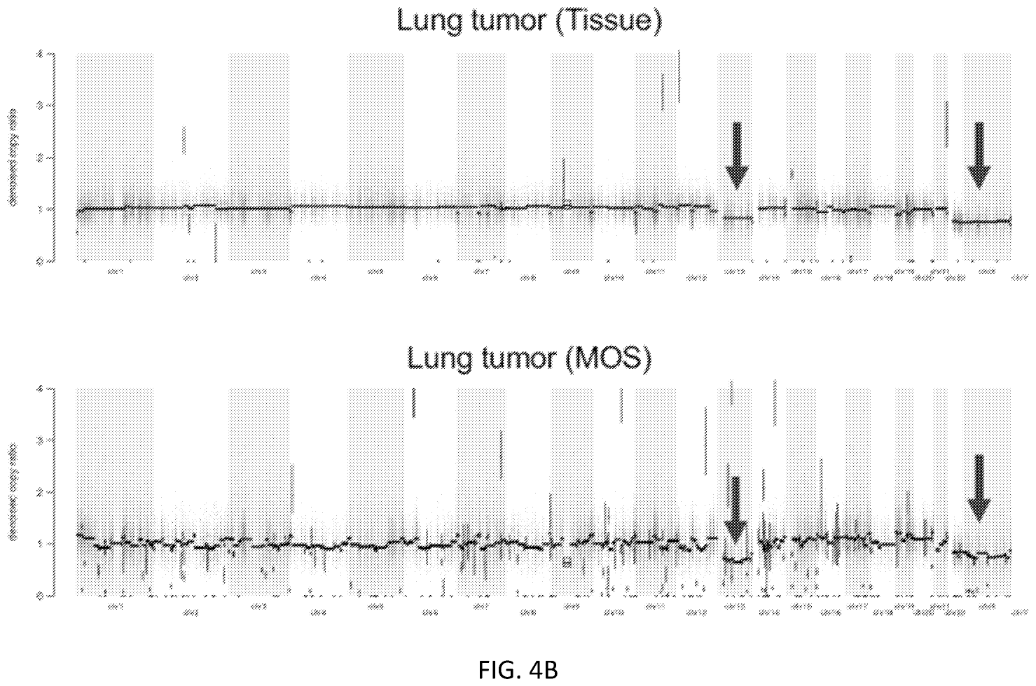

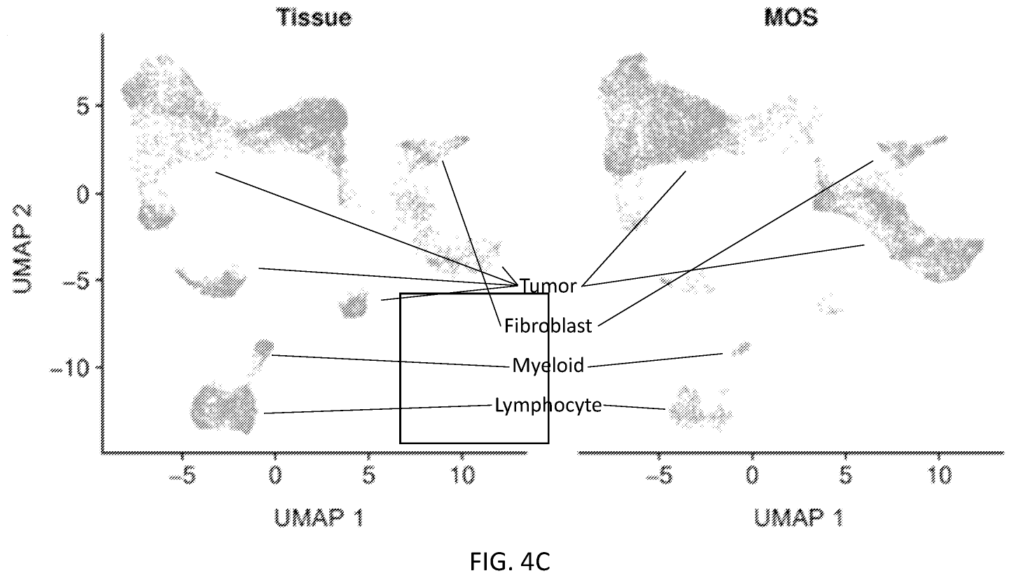

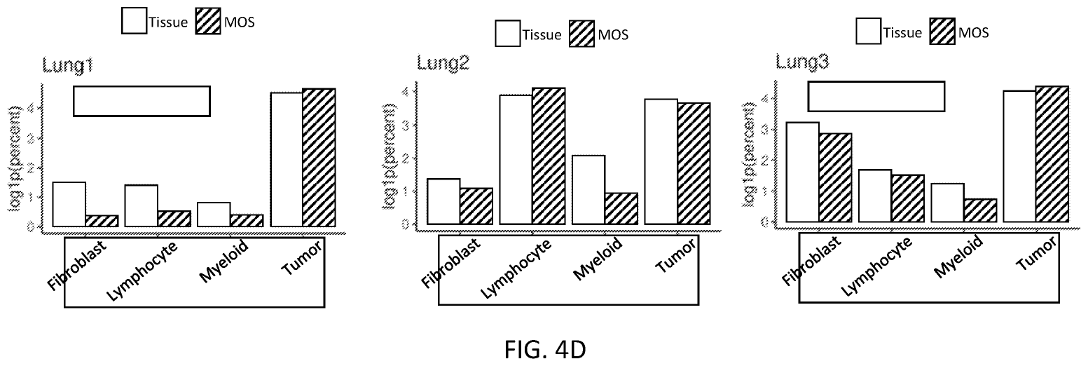

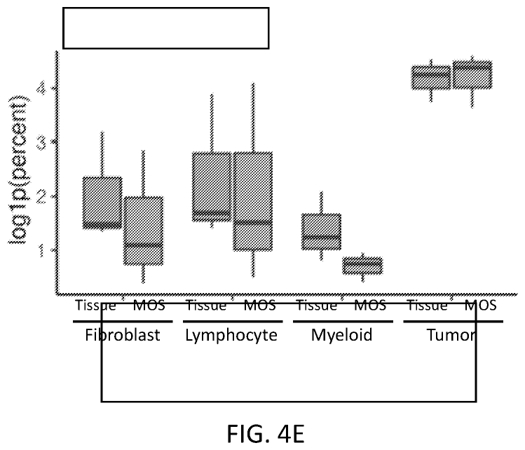

Since the tumor microenvironment, and particularly the immune components, can affect cancer therapy, studies were conducted to characterize the stromal components of patient-derived MOS. The focus was on lung tumor due to its response to immunotherapy, but renal, breast, CRC and ovarian tumors were also characterized to lesser degrees. MOS at a density of 30 tumor cells per MOS were generated in 70% MATRIGEL® diluted in culture medium, and bulk organoids were concurrently established using the same density of cells for comparison. Representative pictures of MOS generated from each tumor type, as well as H&E staining from each tumor tissue and MOS, are shown in FIGS. 3C, 3D, and 4A. Formation and growth of MOS and bulk organoids at days 2, 5, and 7 were comparable (FIG. 3E).

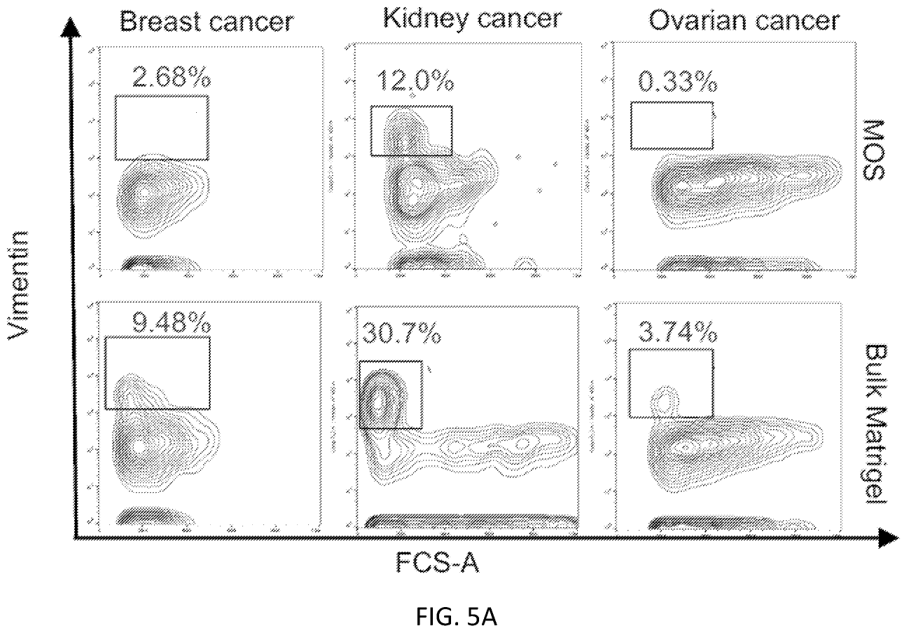

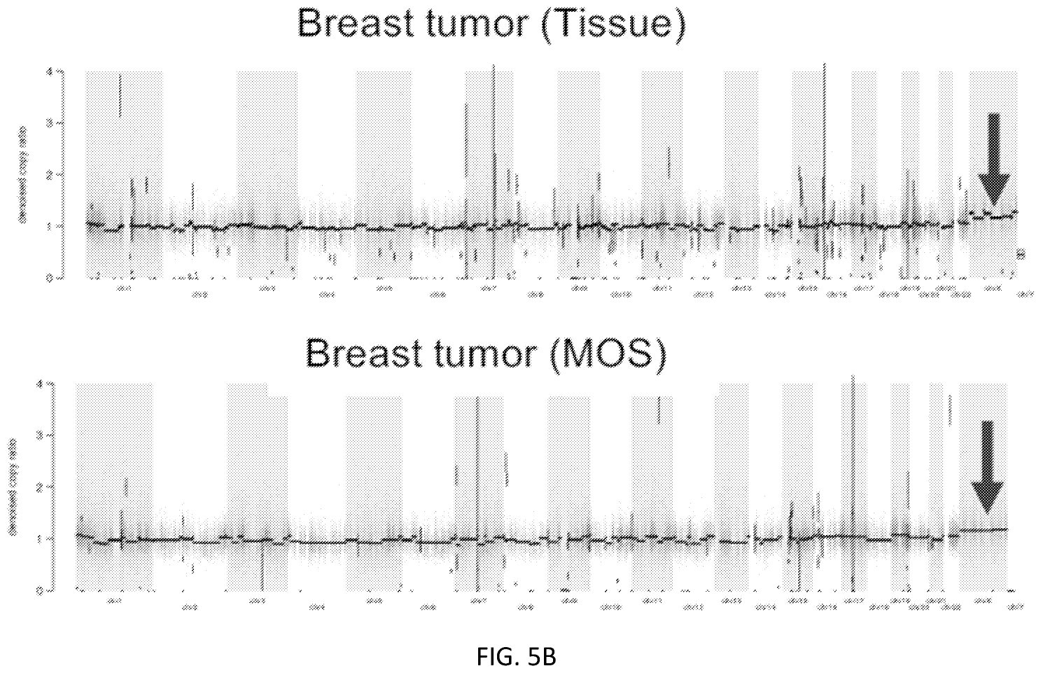

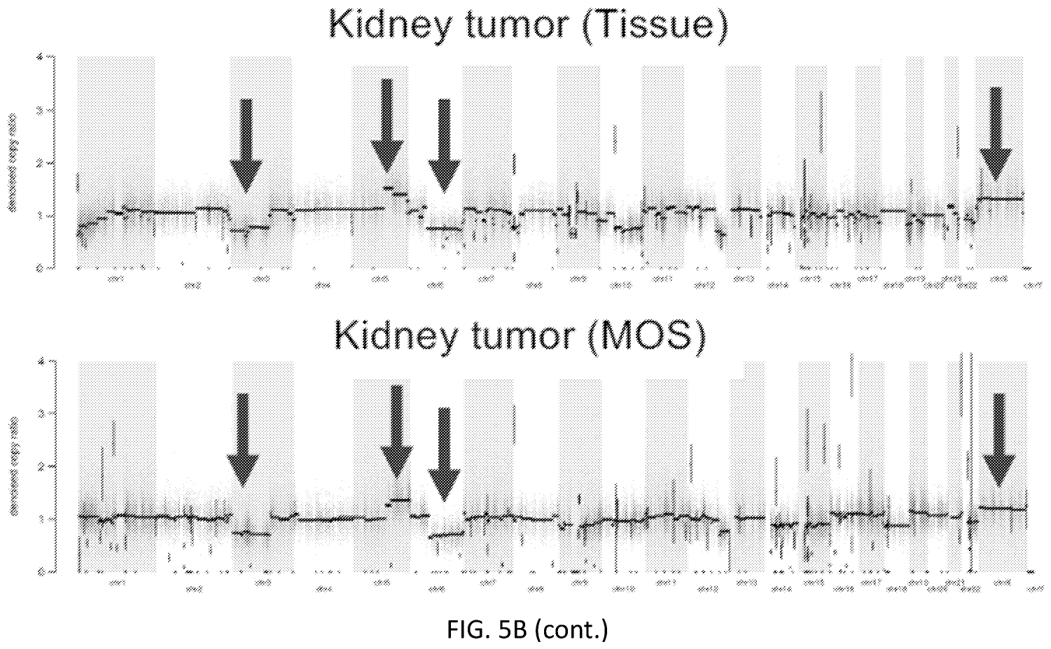

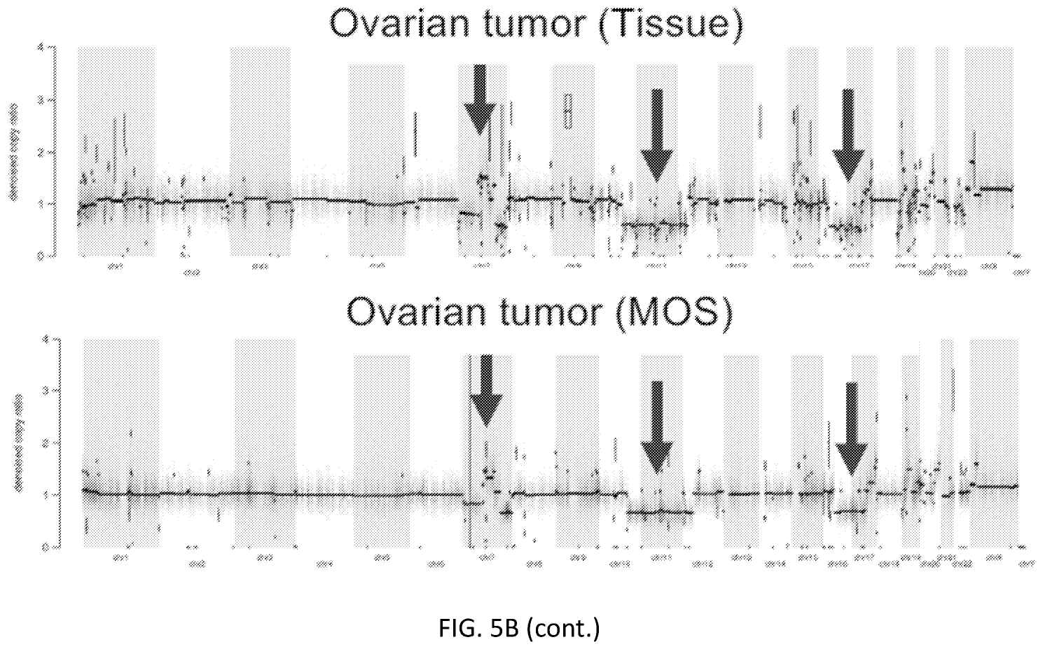

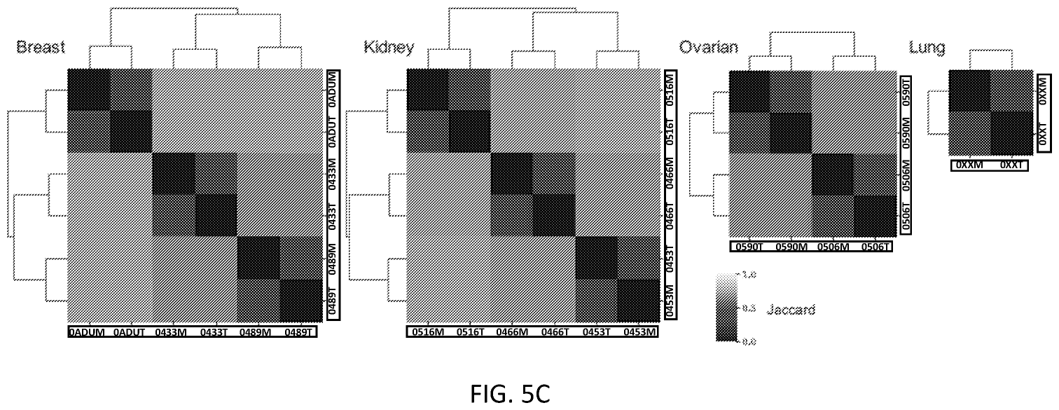

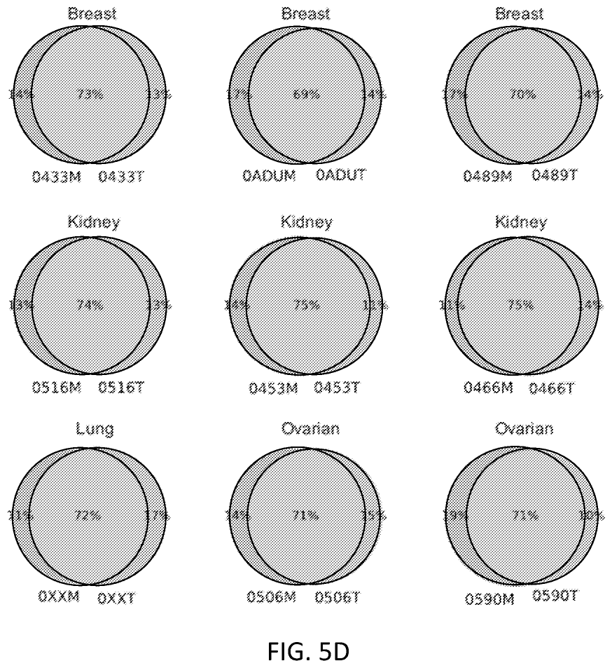

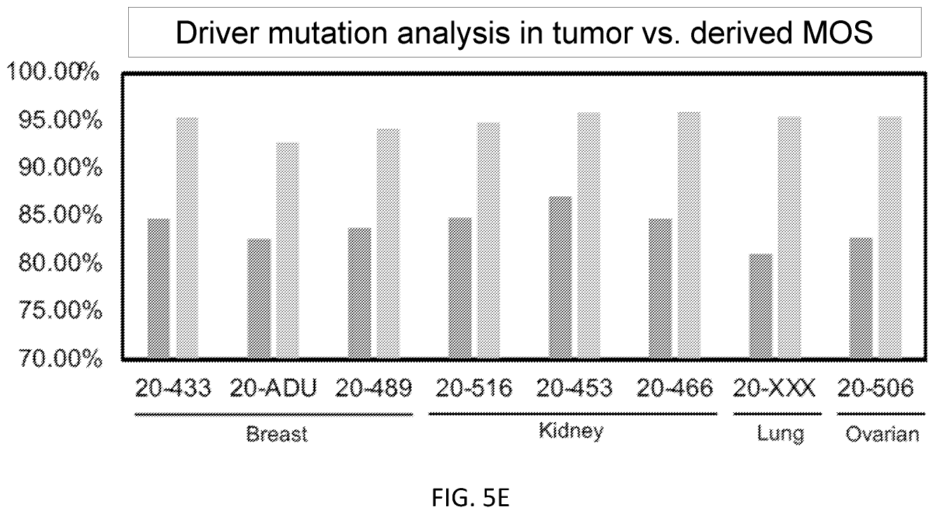

Overgrowth of fibroblasts is often a challenge for establishing organoids from clinical samples of certain cancer types. The number of fibroblasts in MOS and bulk organoid cultures between days 7-9 were compared. Fewer fibroblasts were observed in MOS compared to bulk organoid cultures (FIG. 3F), as confirmed by flow cytometry analysis of Vimentin expression (FIG. 5A). Rapid, high-throughput chemotherapeutic drug screening was then performed on MOS generated from lung, ovarian, and kidney cancer patients, and sensitivities to commonly used agents in the treatment of these cancers was measured.

Whole exome sequencing of MOS was compared to the matched original tumor specimen to determine if genomic alterations were maintained (TABLE 4). First, copy number variations (CNVs) were characterized. A similar pattern of amplifications and losses were seen in MOS and original tissue from lung cancer (FIG. 4B) and other cancer types (FIG. 5B). Second, somatic mutations in the genomes of matched MOS and original tumor samples were characterized. For each cancer type, mutation profiles for matched tissue specimen and MOS were highly correlated, while unmatched samples were not (FIG. 5C). Variants were common between tissue specimens and matched MOS (FIG. 5D). Driver mutations were largely consistent between tissue specimens and MOS among commonly affected genes for each cancer type, with sensitivity (mutations detected in tumor tissue also detected in MOS) of 85%±0.007 and specificity (mutations absent in tumor tissue also absent in MOS) of 95%±0.003 (FIG. 5E). These findings suggest that MOS largely capture the genomic profiles of patient tumors from which they are derived.

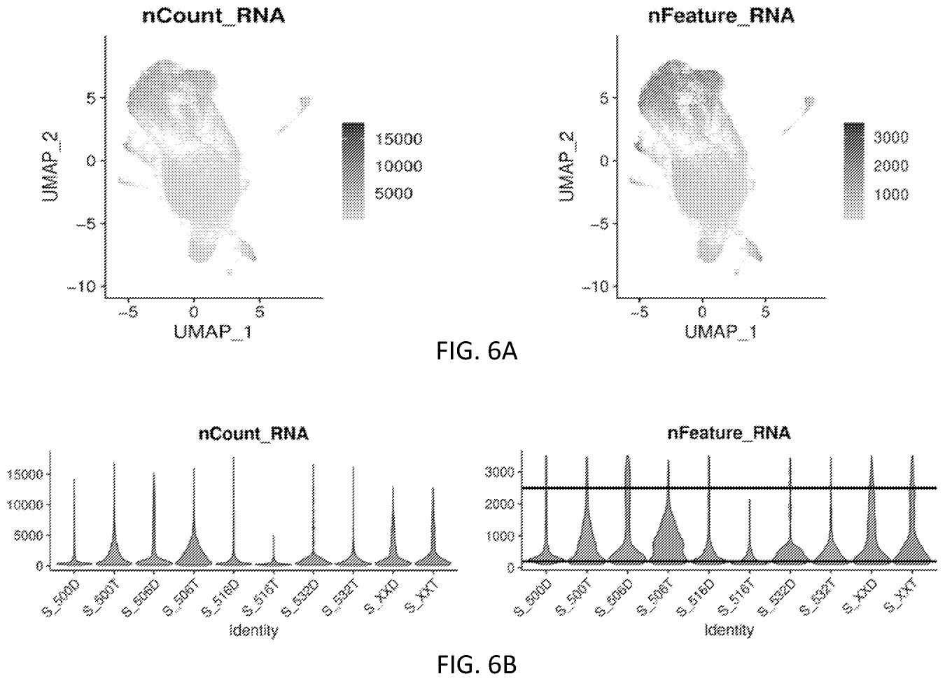

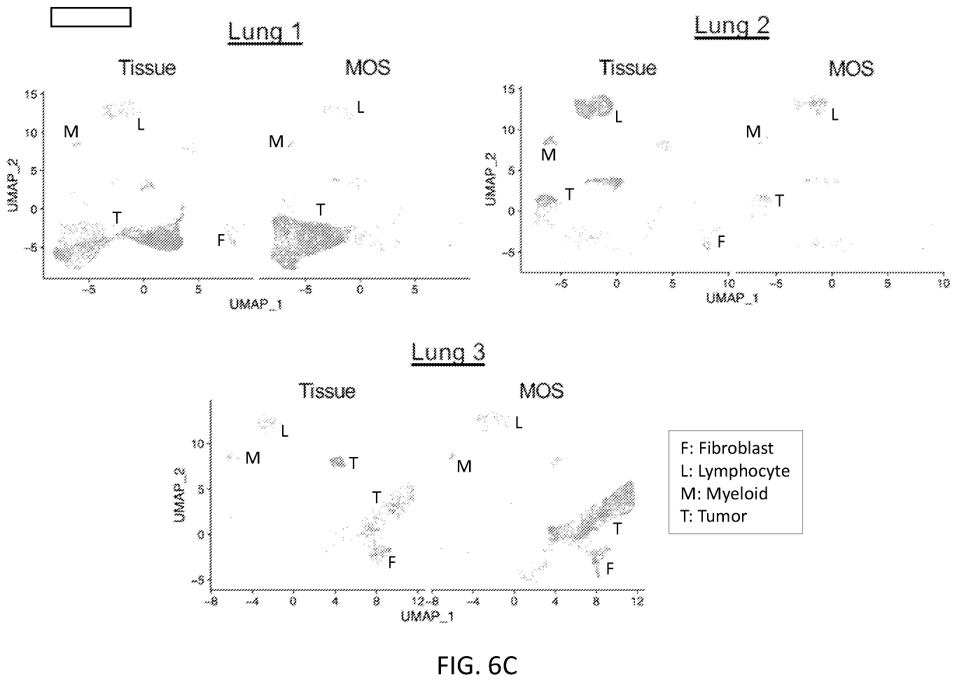

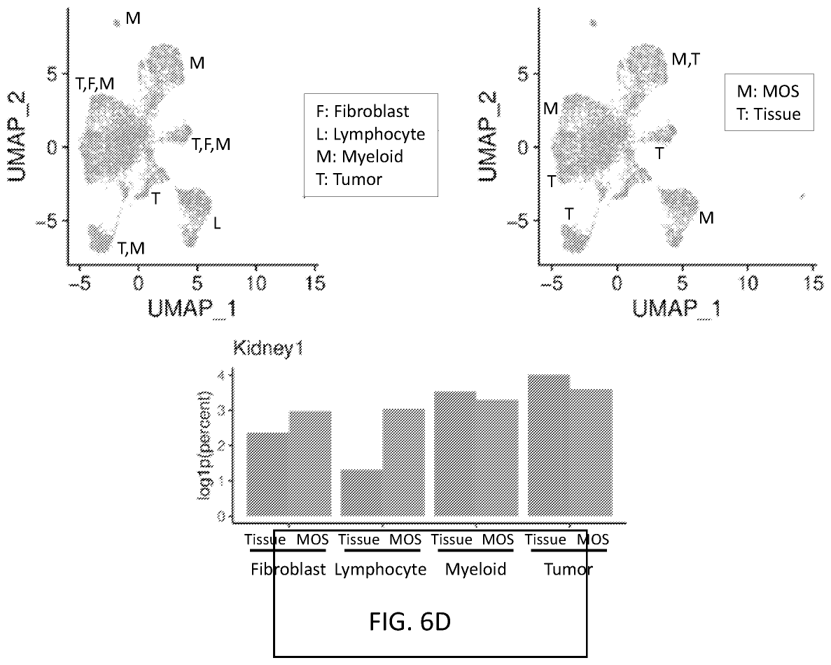

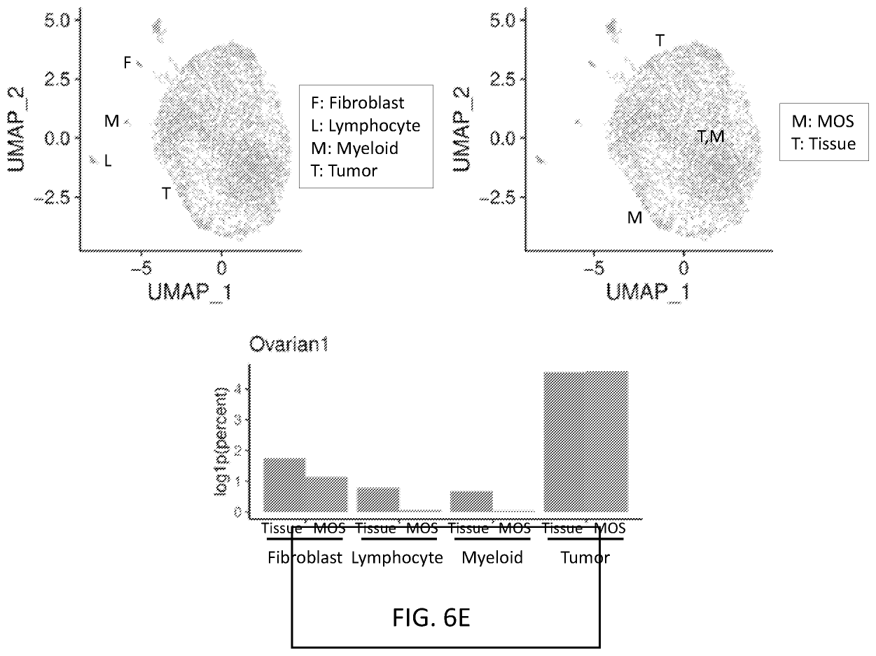

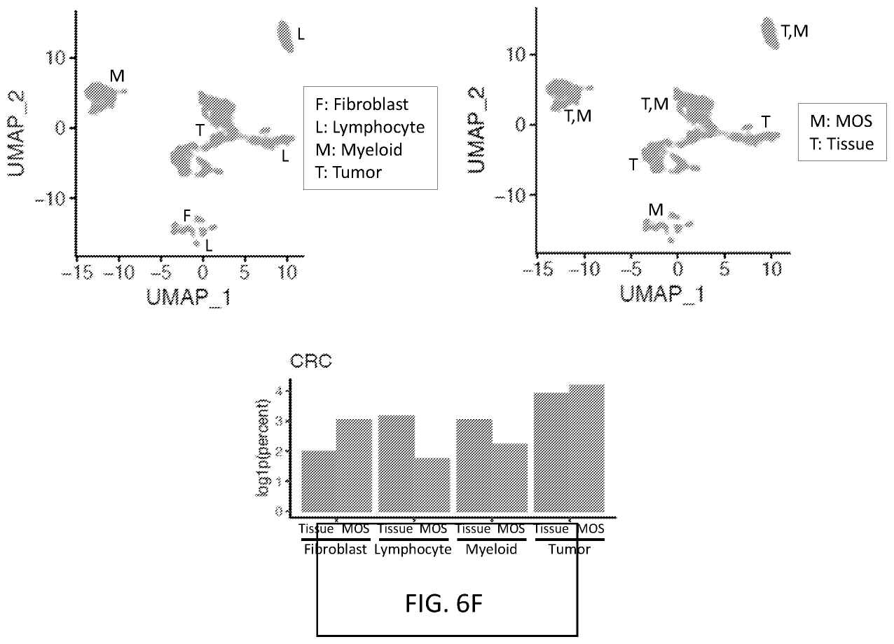

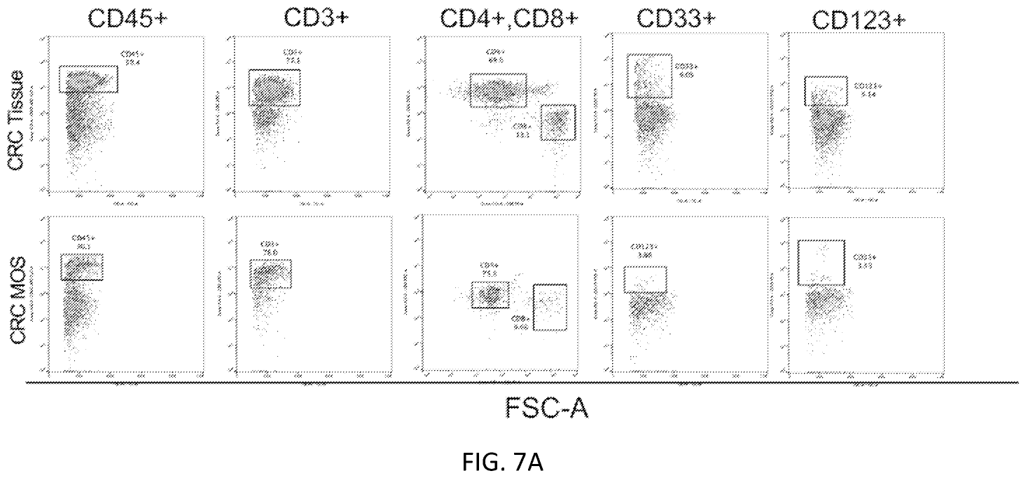

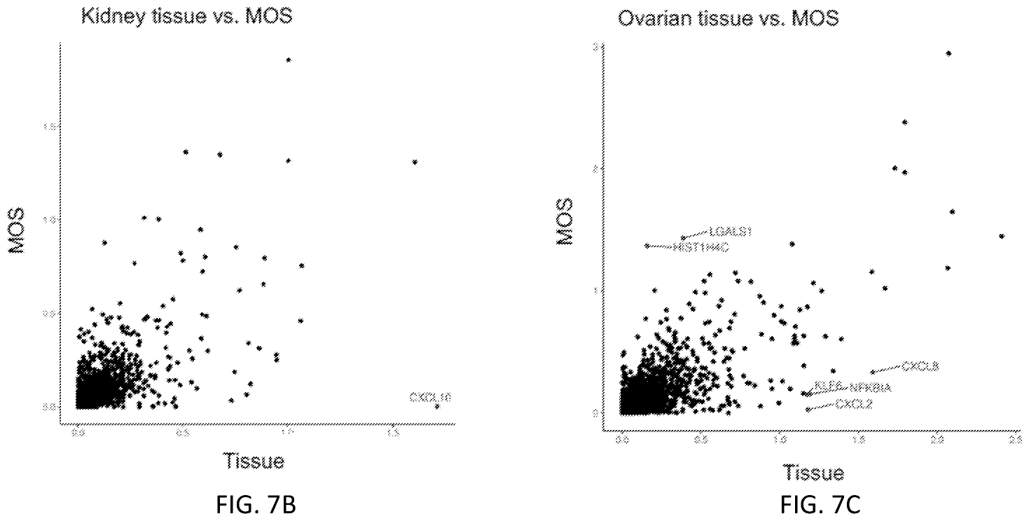

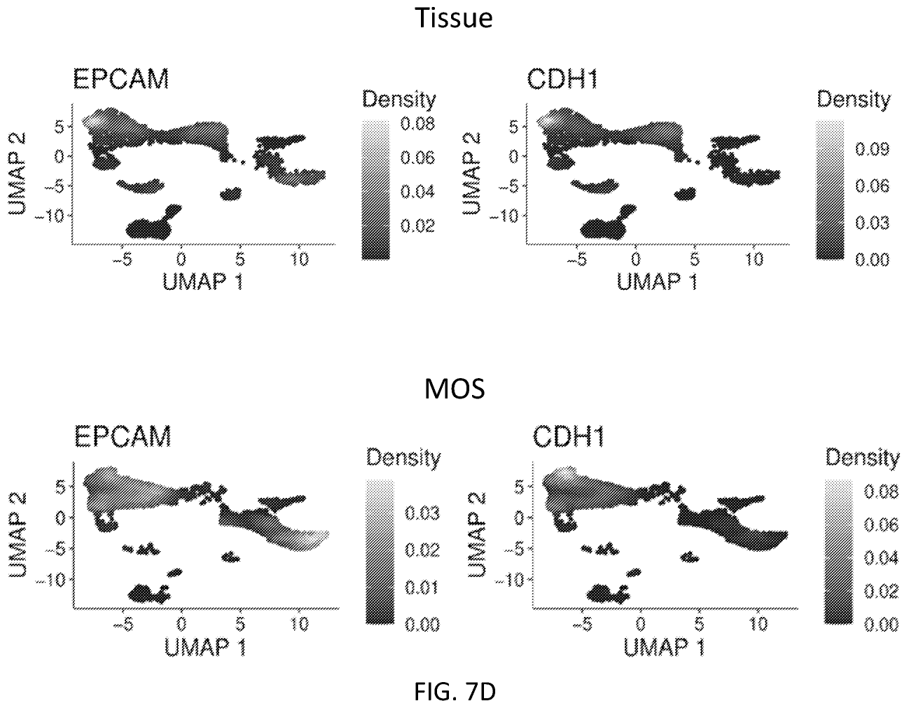

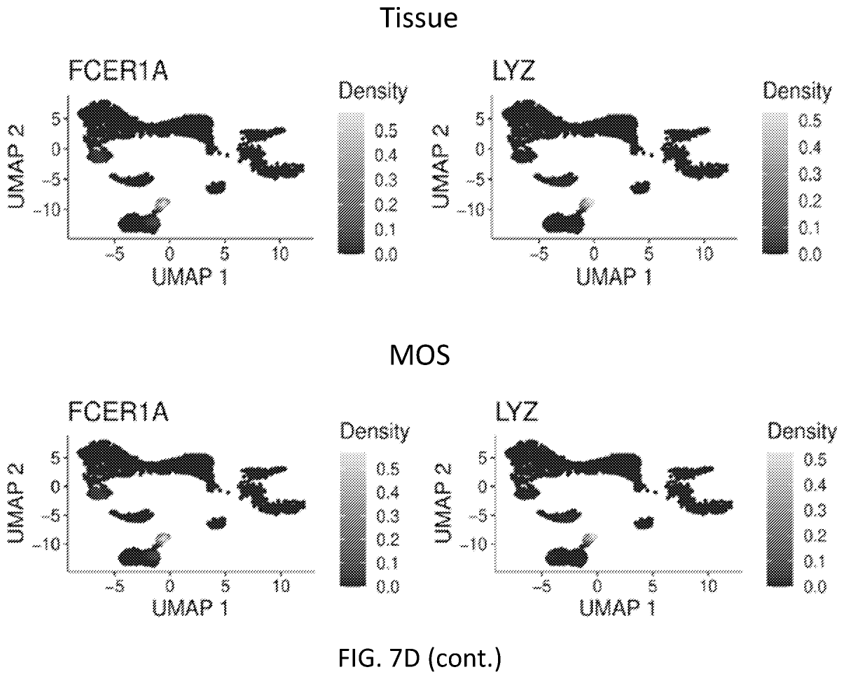

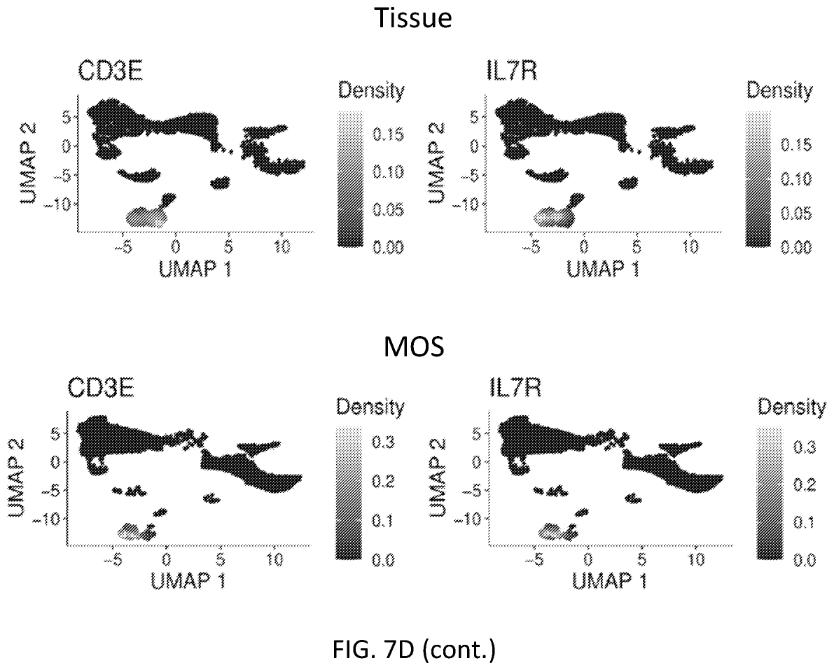

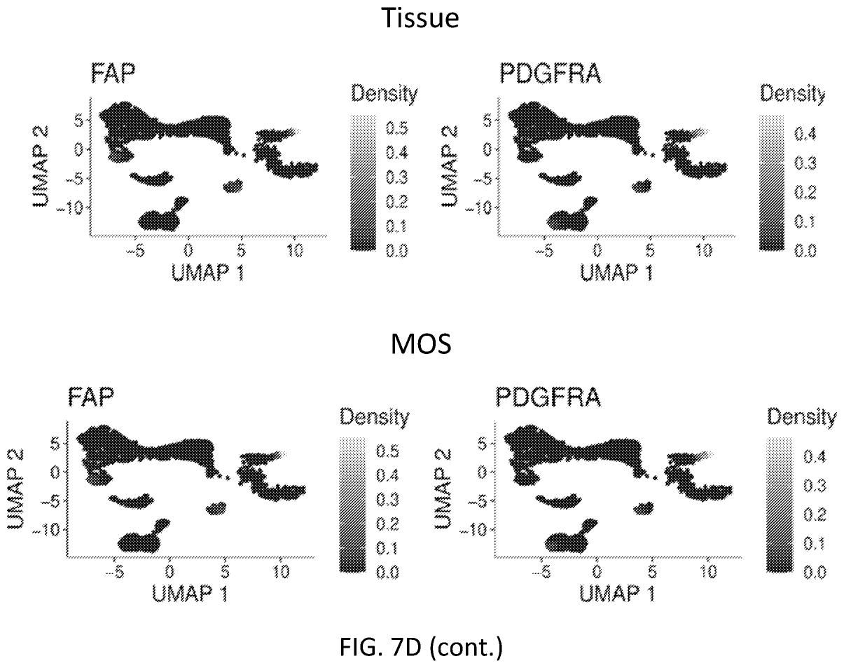

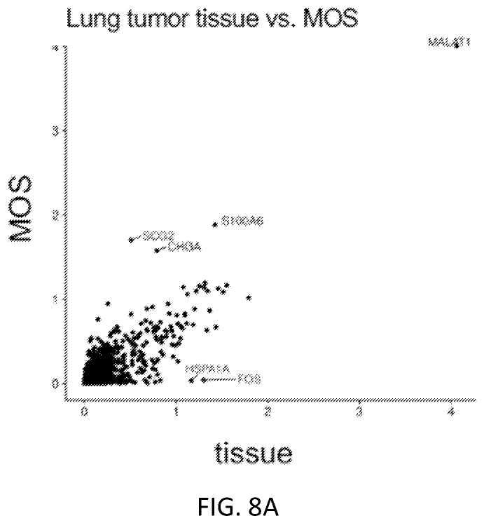

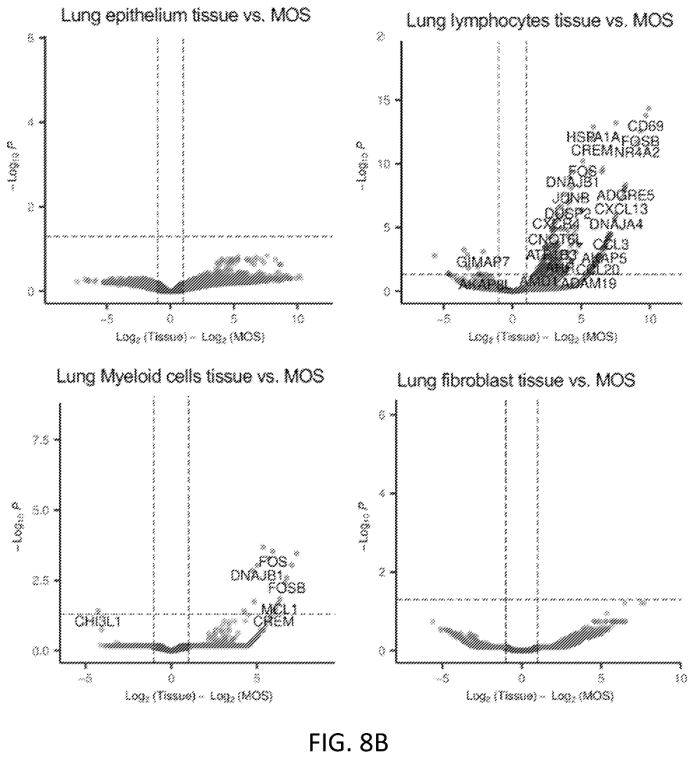

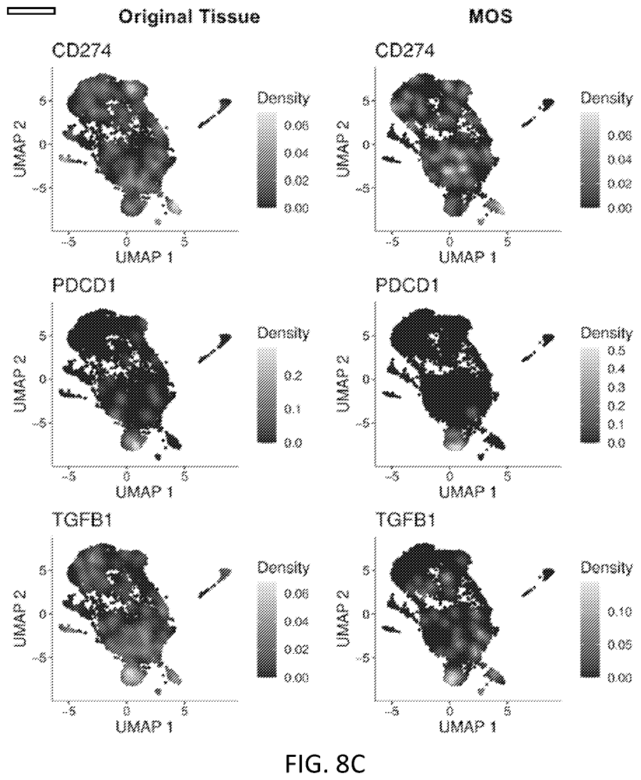

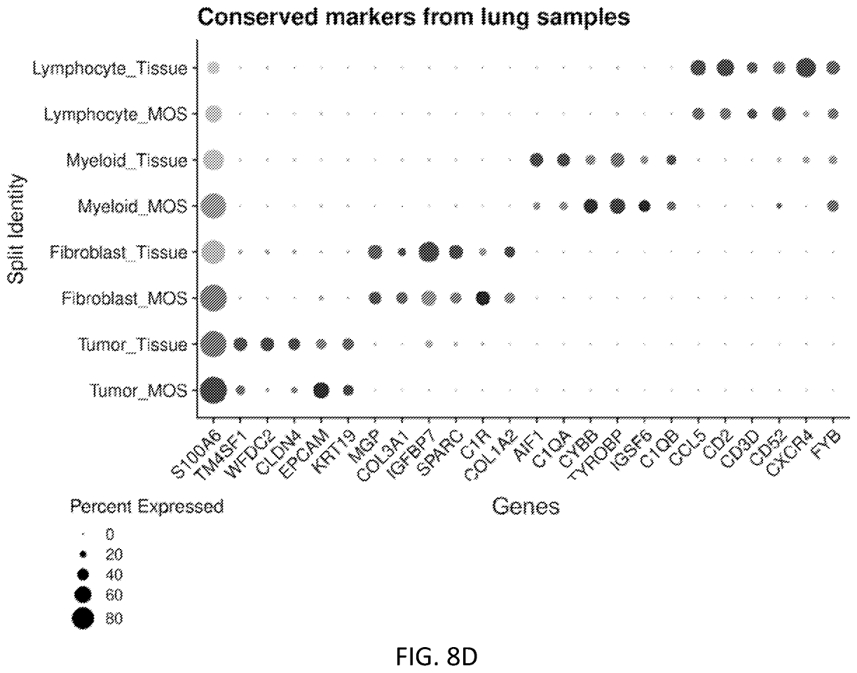

To compare the tumor and stromal cell types between tissue and derived MOS, single cell RNA sequencing (Macosko et al., Cell 161, 1202-1214, 2015) was performed on six pairs of matched patient tumor specimens (three lung cancers, one kidney cancer, one ovarian cancer, and one CRC) and derived MOS (7-9 days). QC summary is shown in FIGS. 6A and 6B. Cells from all three lung tumor samples were clustered using UMAP reductions into four groups marked as tumor cells, cancer-associated fibroblasts, and either lymphoid or myeloid immune cells, which were concordant between tissue and MOS (FIG. 4C) with comparable relative abundance levels (FIGS. 4D and 6C). Similar single cell RNA-seq analyses were performed on the kidney cancer, ovarian cancer and CRC pairs (FIGS. 6D-6F). The presence of major immune cell populations in CRC MOS was confirmed by flow cytometry analysis (FIG. 7A). Pseudo-bulk analysis showed comparable overall gene expression levels in each of these cell populations between primary tissue and MOS (FIG. 4E), with relatively few differentially expressed genes (FIGS. 7B, 7C, and 8A). Analysis of each cell type in lung tumor pairs revealed that lymphoid cells had more differentially expressed genes than the other cell types (FIG. 8B).

Additionally, expression patterns of immunosuppressive markers were largely consistent between lung tumor tissue and MOS. Cell type-specific gene marker expression was visualized using UMAPs following automated cell type labeling with the SingleR package. CD274 (PD-L1) was primarily expressed in tumor and myeloid cell clusters, while PDCD1 (PD-1) and TGFB1 (TGF-β) had elevated expression in lymphoid cells (FIG. 8C). The top five genes with the highest log-fold change enrichment in each cell type were visualized to confirm concordant expression for each cell type and sample preparation (FIG. 8D). These conserved markers, including key cell type-specific markers EPCAM, PDGFRA, LYZ, and CD3E for tumor cells, fibroblasts, myeloid cells, and lymphoid cells, respectively, were largely consistent between tissue and derived MOS (FIG. 7D).

### Immune Cells Preserved in MOS Respond to Immune Therapy

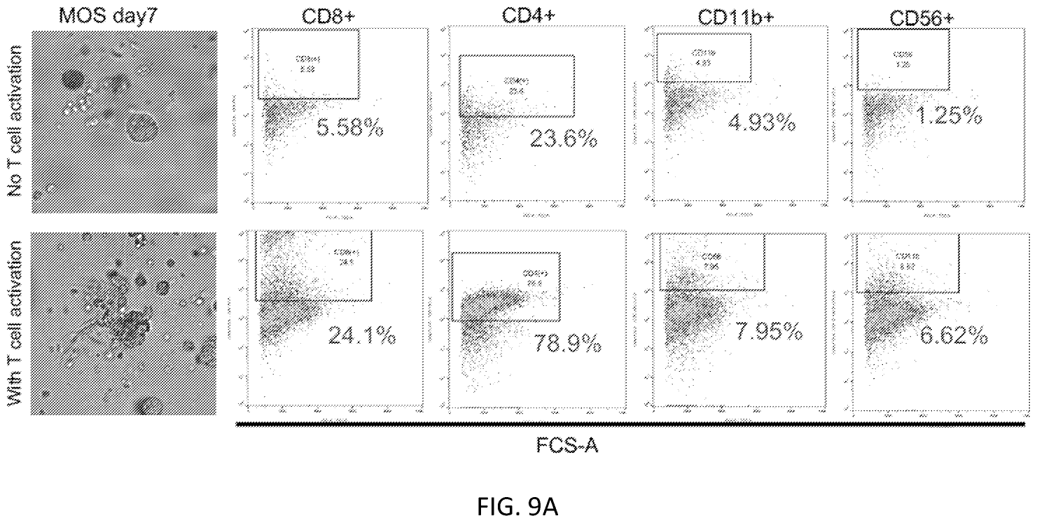

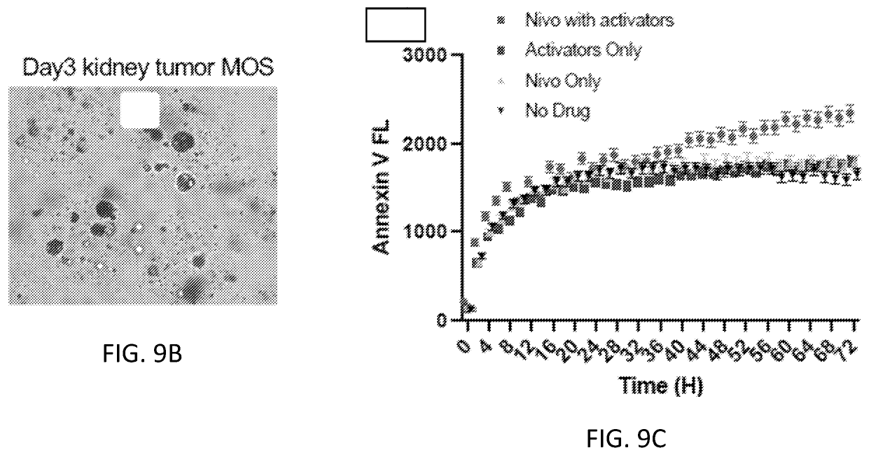

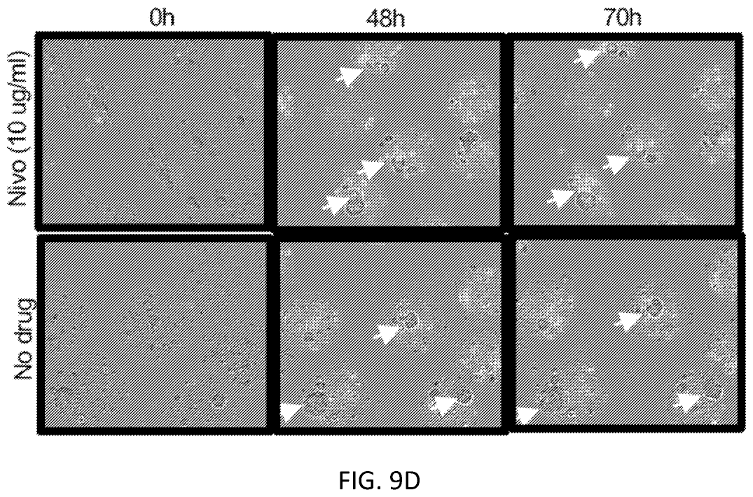

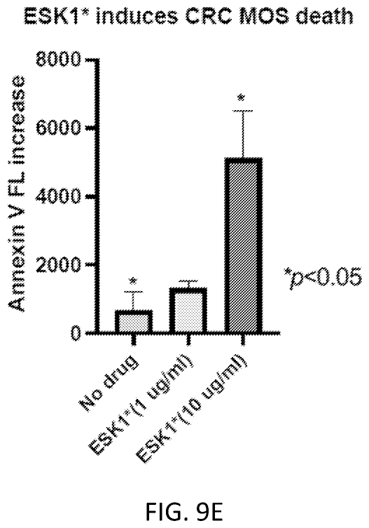

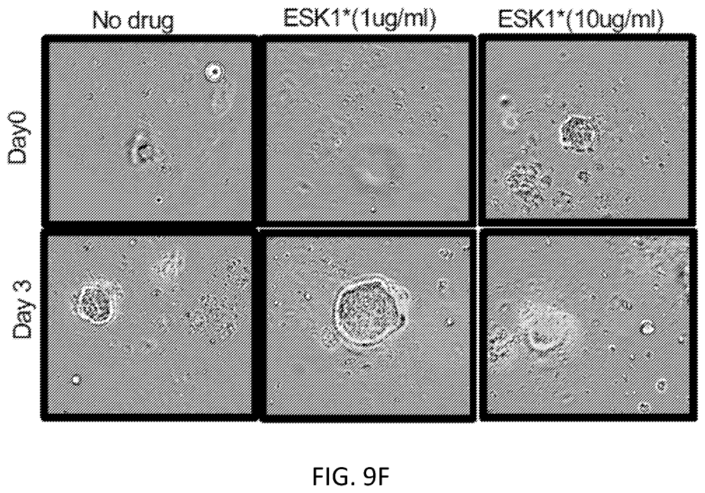

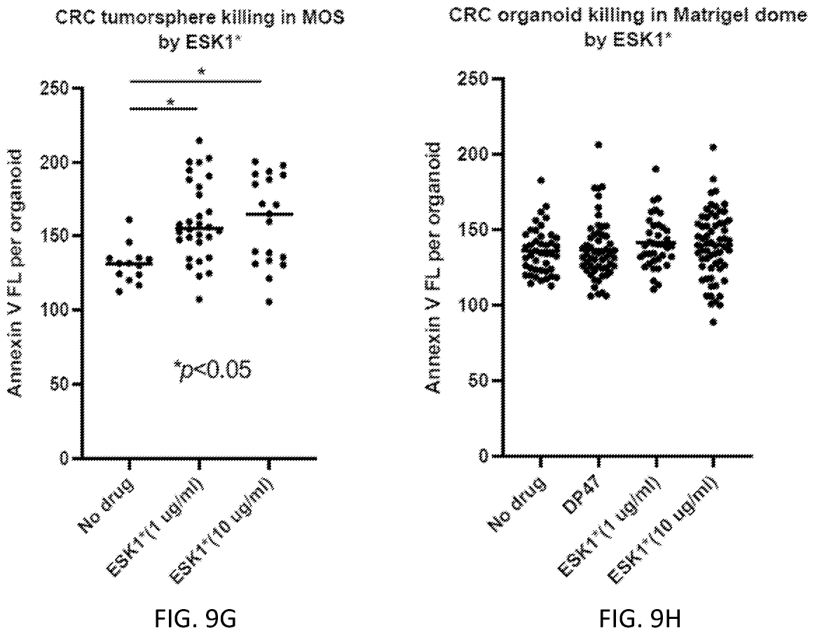

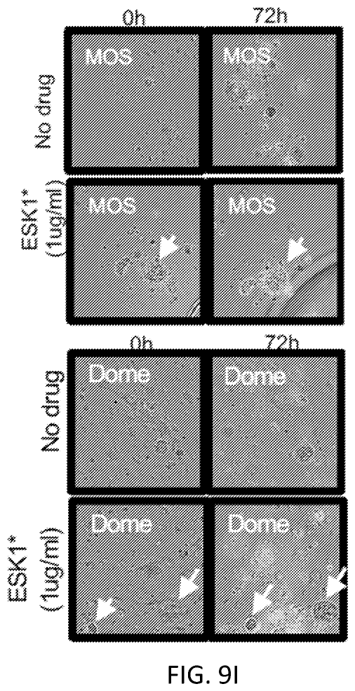

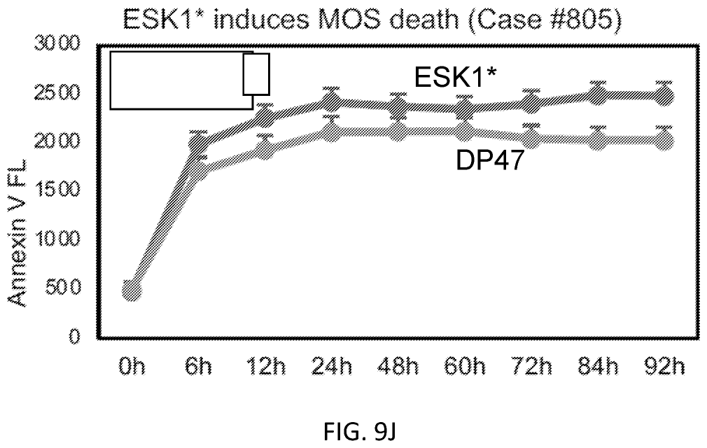

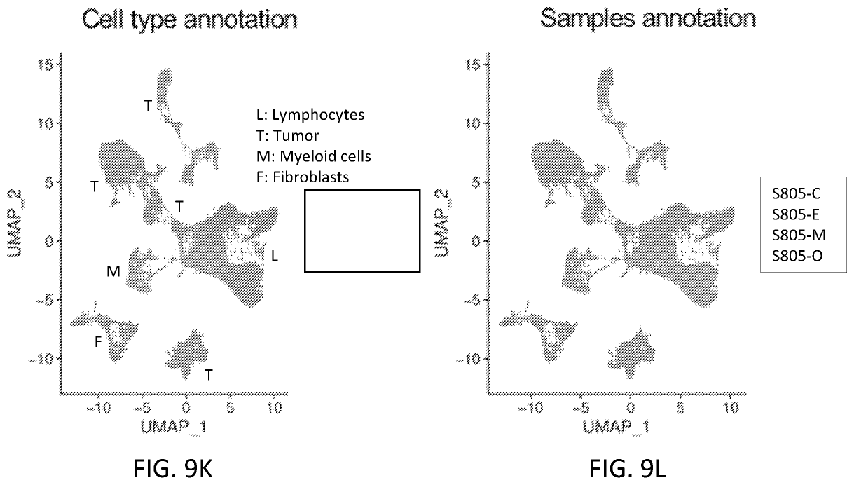

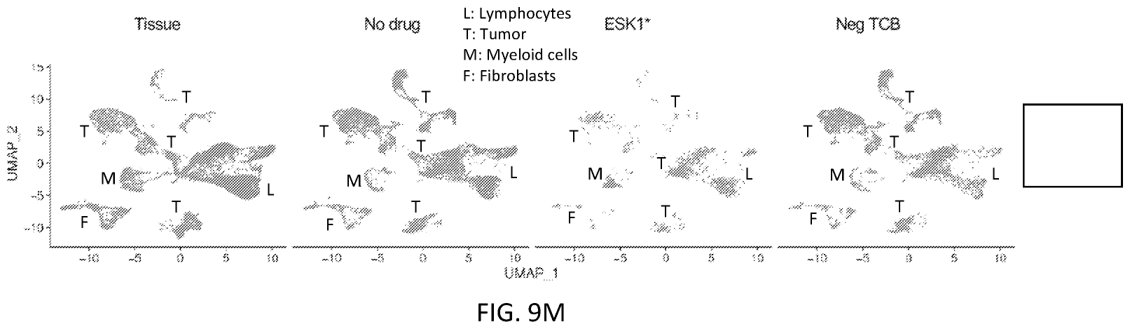

Studies were conducted to examine whether the patient immune cells in MOS were functional and responsive to IO therapies. Addition of anti-CD3 and anti-CD28 antibodies to MOS medium increased CD4+, CD8+, and CD56+ (Natural Killer cell marker) populations and had a modest effect on the CD11b+ (dendritic cell marker) populations (FIG. 9A), suggesting that resident immune cells encapsulated in MOS are viable and responsive to immune stimulation.

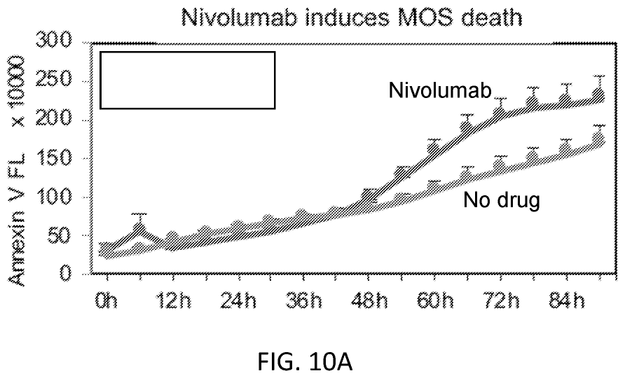

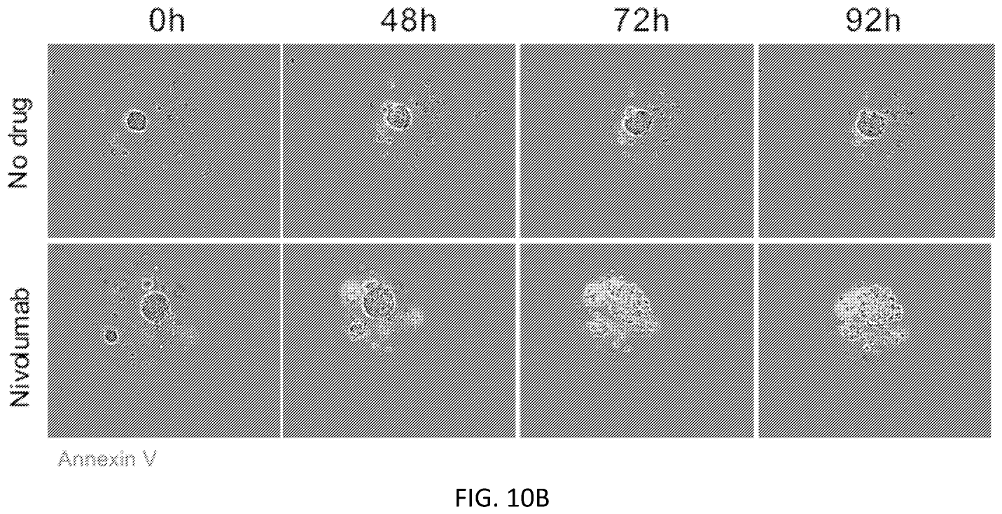

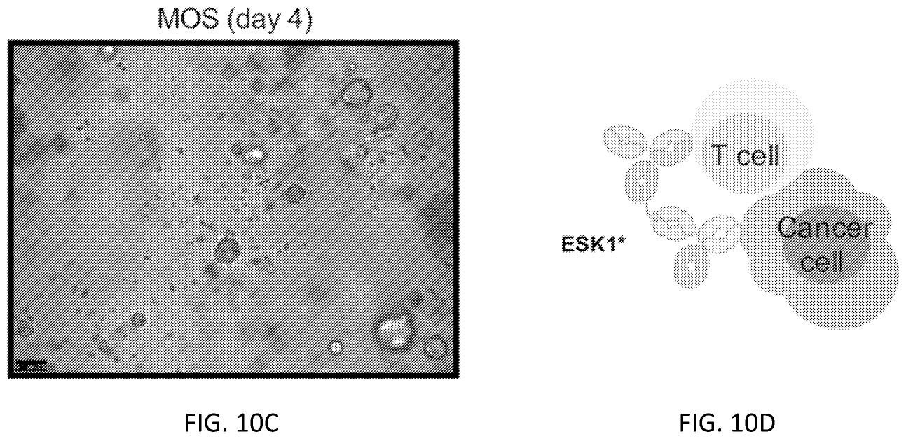

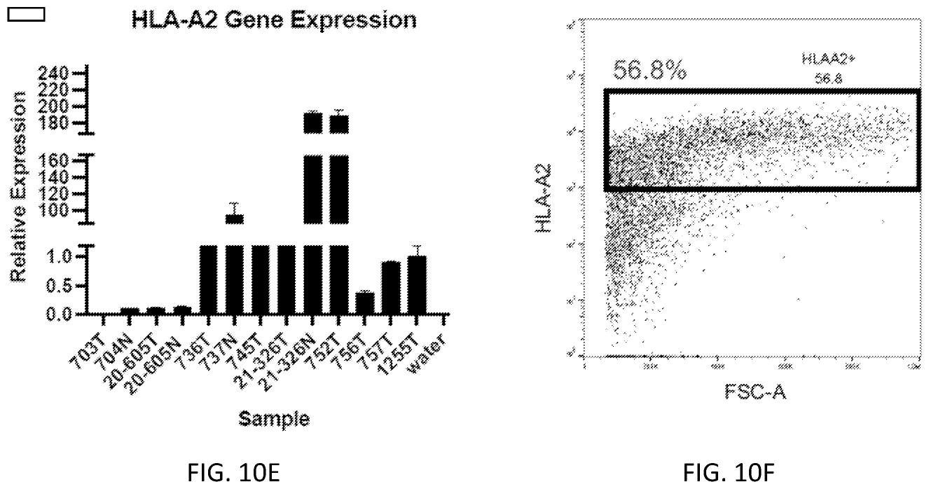

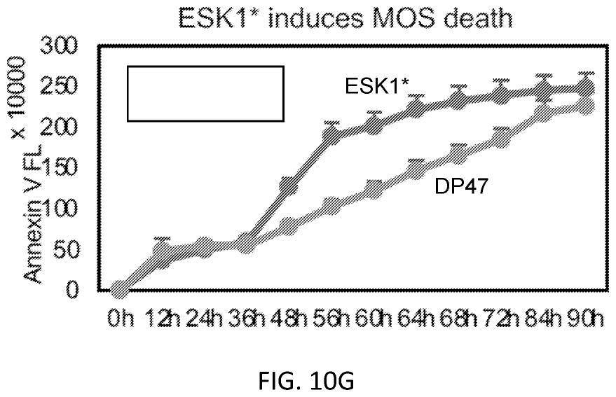

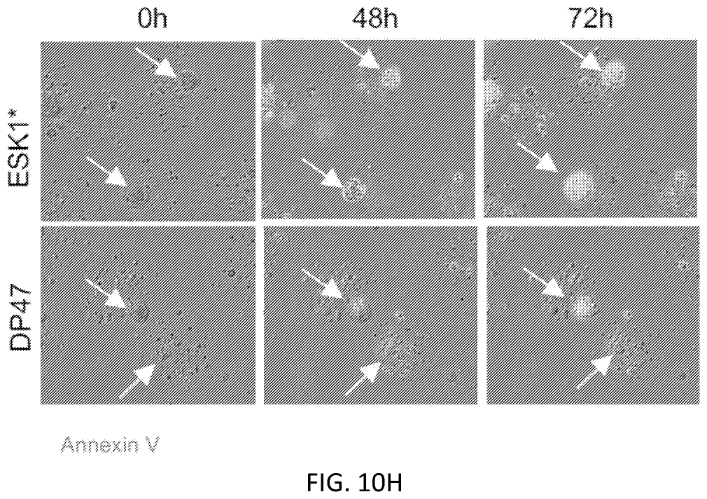

Immune checkpoint inhibitors, specifically those targeting the programmed cell death-1 (PD-1)/programmed cell death ligand-1 (PD-L1) axis, have demonstrated promising activity in non-small cell lung cancer (NSCLC) (Han et al., Am J Cancer Res 10, 727-742, 2020). However, there remains a crucial need for an in vitro assay to better guide IO treatment for patients with advanced NSCLC, as PD-L1 expression and tumor mutation burden do not completely predict patient response. MOS generated from NSCLC patient samples at a density of 30 tumor cells per MOS formed tumorsphere-like structures in the presence of tumor-resident immune cells. MOS (day 4) were then treated with anti-PD1 therapy nivolumab at 10 μg/mL and Annexin V was used to evaluate cell apoptosis. Nivolumab induced death in the tumorspheres within MOS (FIGS. 10A and 10B). The Incucyte measurements also contained background signals outside tumorspheres from cell debris in the MOS microenvironment, giving rise to the rising curves in the control. In MOS (day 3) derived from a kidney cancer patient, nivolumab treatment alone did not enhance killing of tumorspheres inside MOS while a combination of nivolumab and T cell activator enhanced tumorsphere killing (FIGS. 9B-9D).

Intracellular antigens presented on the cell surface by human leukocyte antigen (HLA) molecules have been targeted by T cell-based therapies. Studies were conducted to test whether a non-selective HLA-A*02/WT1 targeting antibody ESK1* (ESK-1 tumor binder, Roche proprietary CD3), a T-cell receptor mimic monoclonal antibody (mAb) that binds both human leukocyte antigen HLA-A2/WT1 and CD3, could induce cytotoxic T cell (CTL)-mediated killing in MOS derived from patient lung tumor (FIGS. 10C and 10D) (Dao et al., Nature Biotechnol 33, 1079-1086, 2015). HLA-A2 genotype was validated by qRT-PCR (FIG. 10E) and flow cytometry (FIG. 10F). ESK1* was compared to negative control DP47, a non-tumor targeted T-Cell bispecific (CD3 arm only) antibody (TCB). ESK1* or DP47 was added into the MOS culture medium (without Y compound) on day 5. ESK1* induced apoptosis (indicated by Annexin V signal) in MOS (FIG. 10G). Although DP47 was also capable of activating T cells via CD3 and causing cell death, ESK1* induced more killing in all eight lung cancer patient cases (FIGS. 10H and 10I).

CRC MOS (HLAA2+) were then treated with ESK1*. MOS were generated at a density of 30 tumor cells per MOS. A higher dose of ESK1* (10 μg/mL) induced more tumorsphere death in MOS than a lower dose of ESK1* (1 μg/mL) (FIGS. 9E and 9F). Quantification of Annexin V fluorescence signals from individual tumorspheres confirmed ESK1-mediated killing in MOS but not in organoids embedded in traditional MATRIGEL® dome (FIGS. 9G-9I).

To further understand how MOS respond to ESK1*, 10X single cell RNA-seq was performed on original lung tumor tissue cells at day 0, and MOS treated with ESK1*, negative TCB (DP47), as well as no added treatment at day 5 (FIG. 9J). The clusters profiled from tissue sample and treated MOS were consistent (FIGS. 9K and 9L). The abundance of each cell type from MOS with DP47 or without treatment were similar to original tissue cells but decreased drastically in ESK1*-treated MOS (FIG. 9M). Collectively, these results suggest that the MOS assay can rapidly assess the impact of IO therapy molecules such as PD-1 blockade and TCB on patient tumor and stromal cells.

### A MOS Potency Assay for T-Cell Therapies

Adoptive T cell therapies (ACT), such as chimeric antigen receptor T-cell (CAR-T) therapy and TIL therapy, have the potential to transform cancer treatment (June et al., Science 359, 1361-1365, 2018; Waldman et al., Nat Rev Immunol 20, 651-668, 2020). However, an area of unmet need is an assay to assess the potency of manufactured T cells against a patient's tumor, which is required by regulatory authorities such as the FDA for approving cell therapies (HHS and FDA, www.fda.gov/files/vaccines,%20blood%20&%20biologics/published/Final-Guidancefor-Industry--Potency-Tests-for-Cellular-and-Gene-Therapy-Products.pdf, 2011). Interferon gamma release has been used to evaluate TILs against patient tumors, but at least four studies have shown that it does not correlate with clinical response (Besser et al., J Immunother 32, 415-423, 2009; Dudley et al., Clin Cancer Res 16, 6122-6131, 2010; Nguyen et al., Cancer Immunol Immunother 68, 773-785, 2019; Radvanyi et al., Clin Cancer Res 18, 6758-6770, 2012). For ACT with TILs, the patient tumor model has to be established rapidly from a fraction of the biopsy (as the majority has to be used to extract and expand TILs), making it particularly challenging.

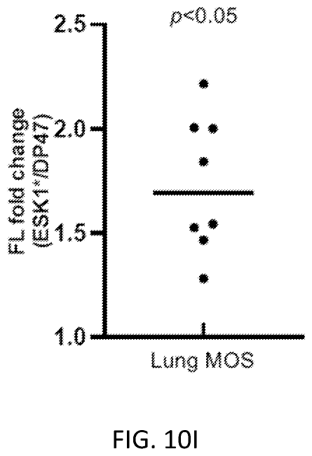

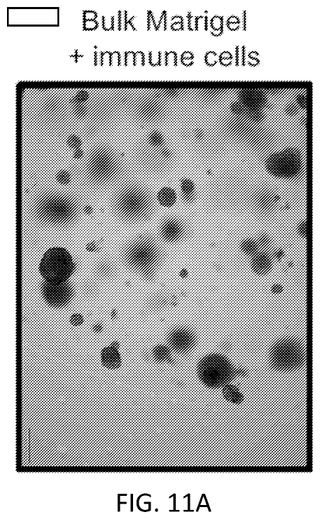

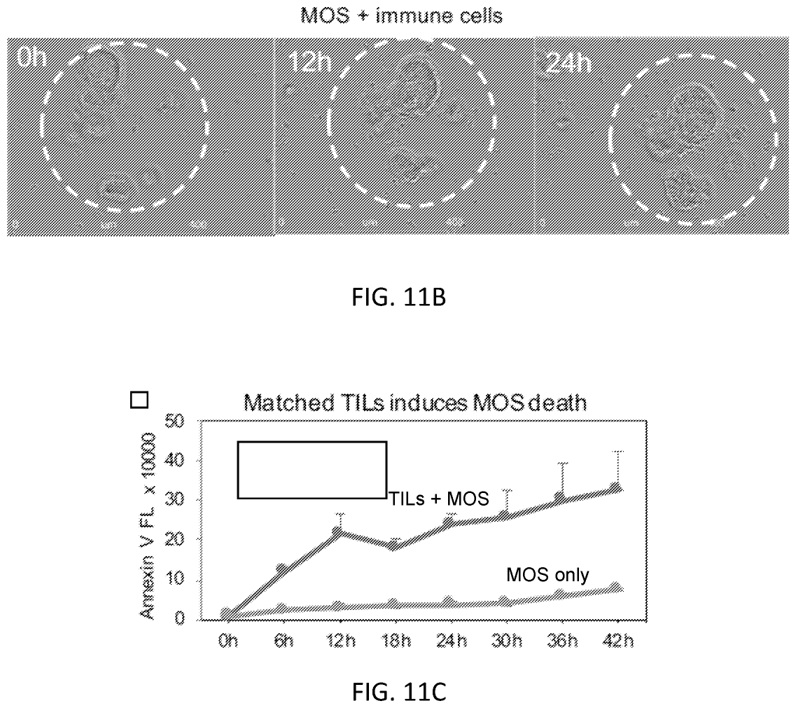

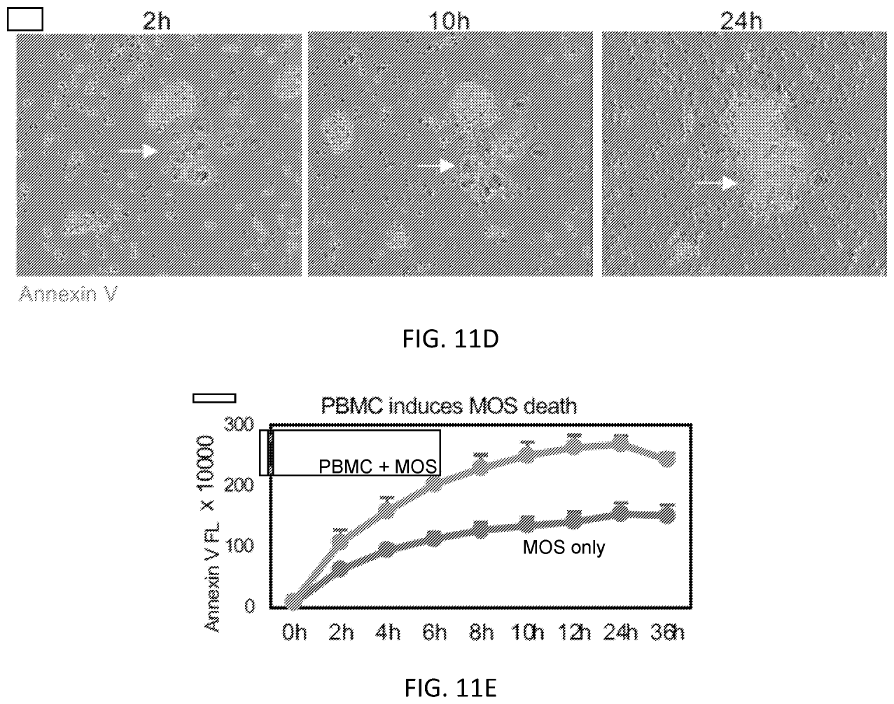

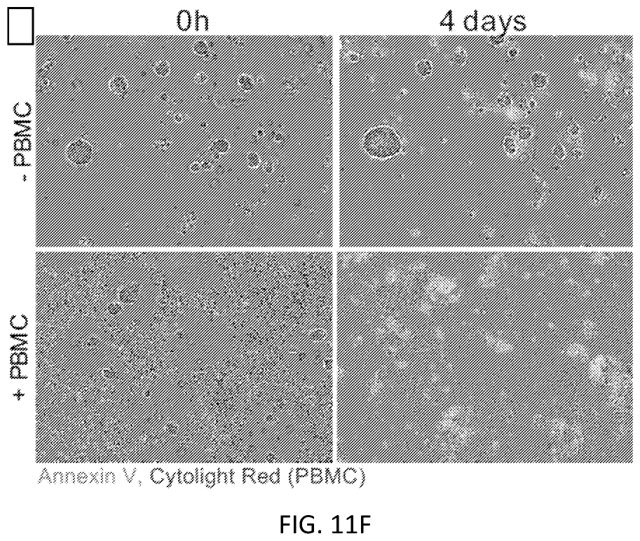

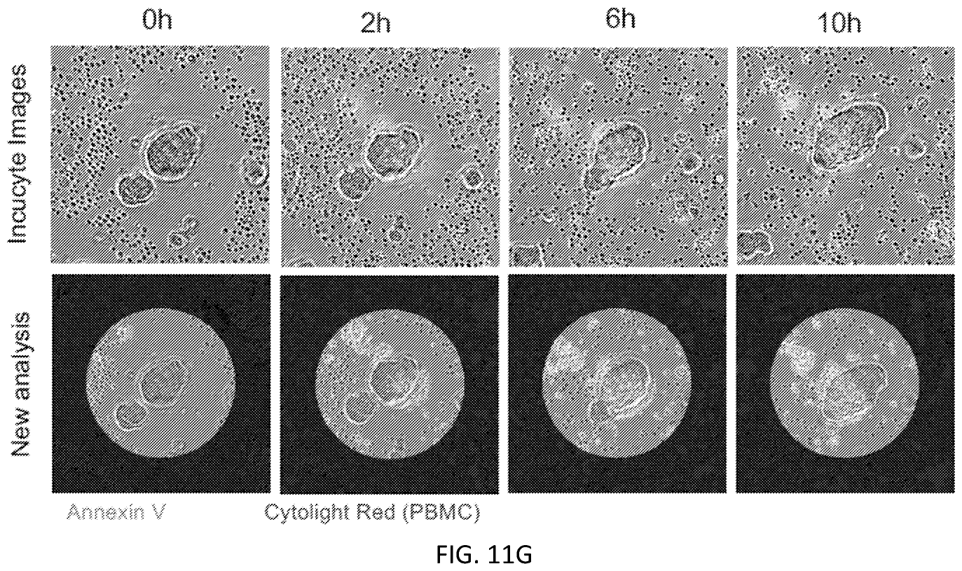

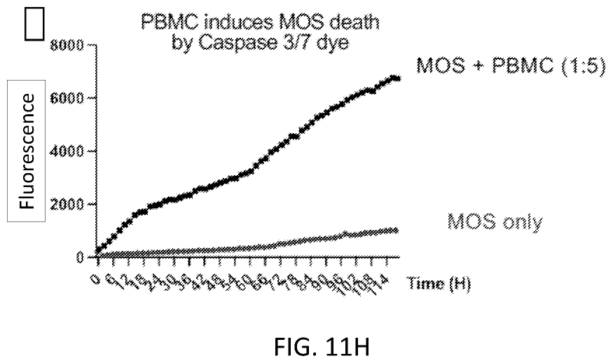

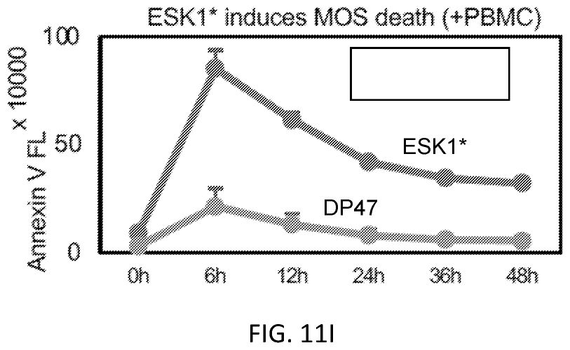

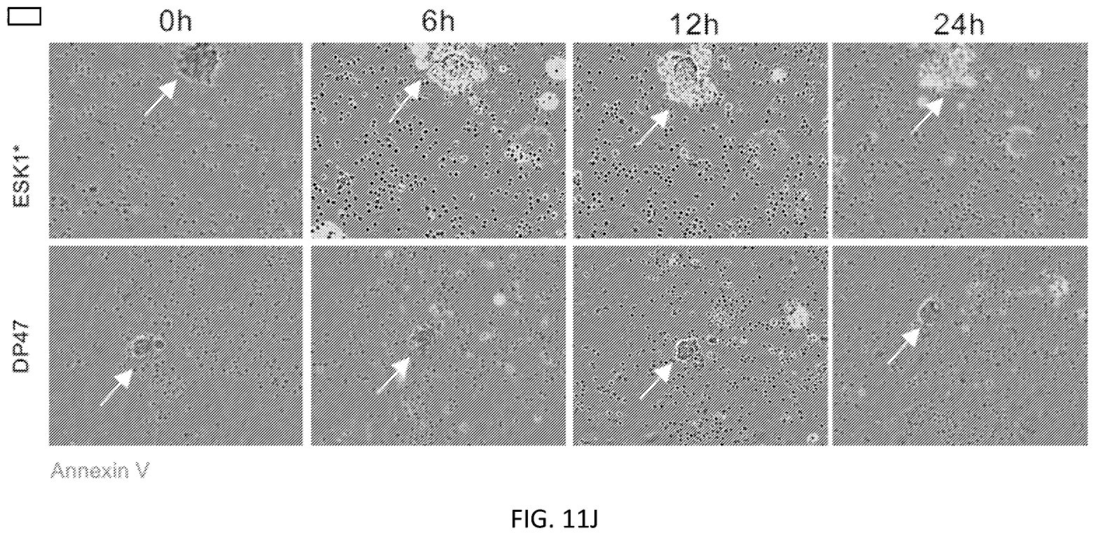

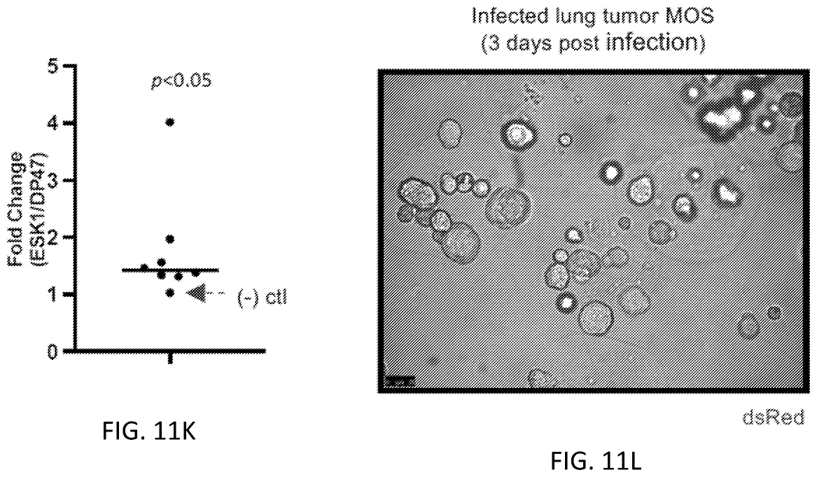

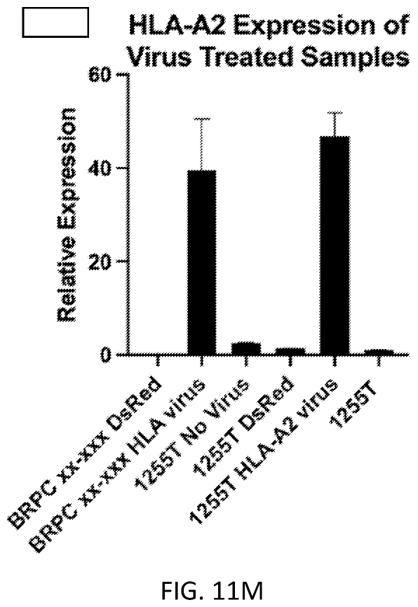

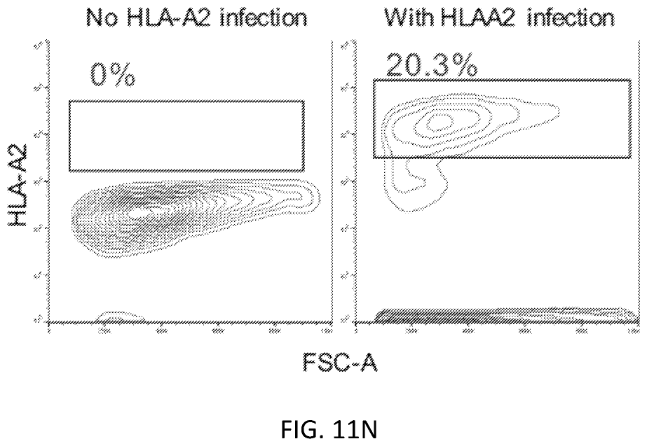

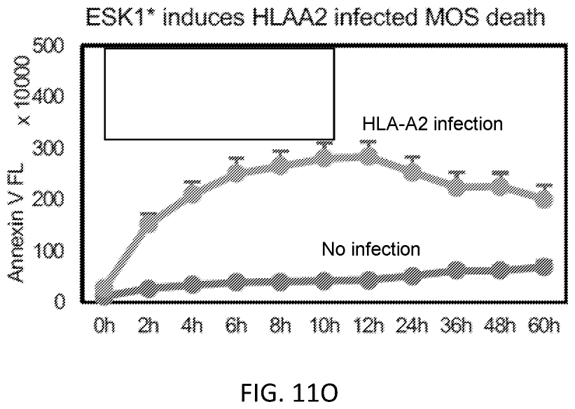

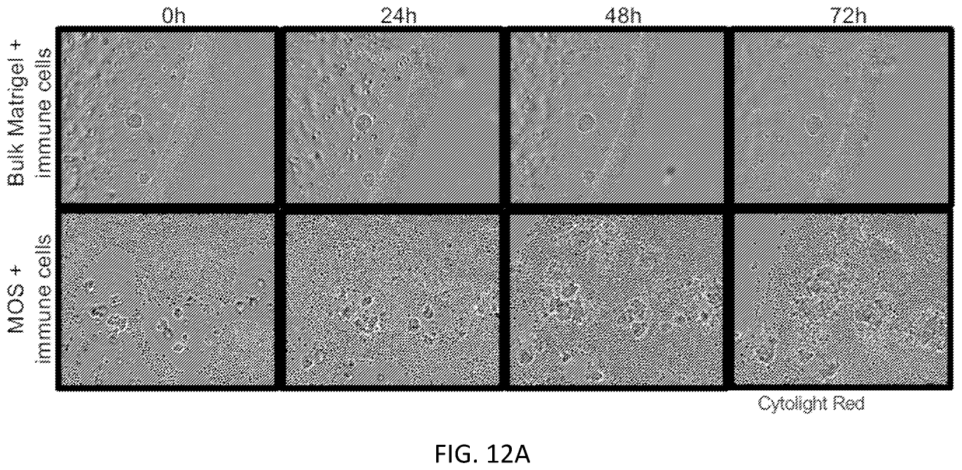

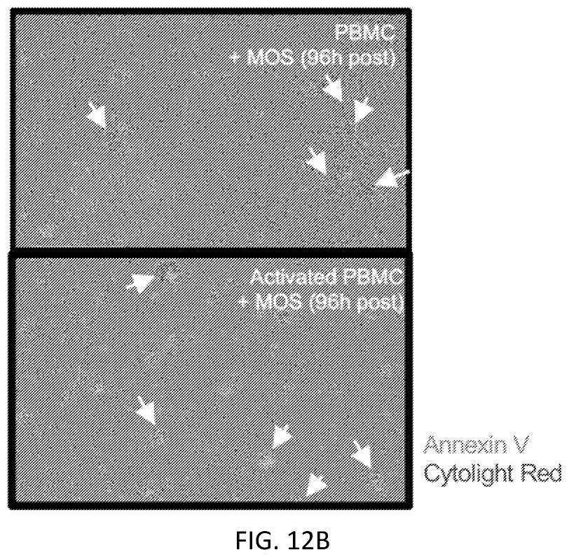

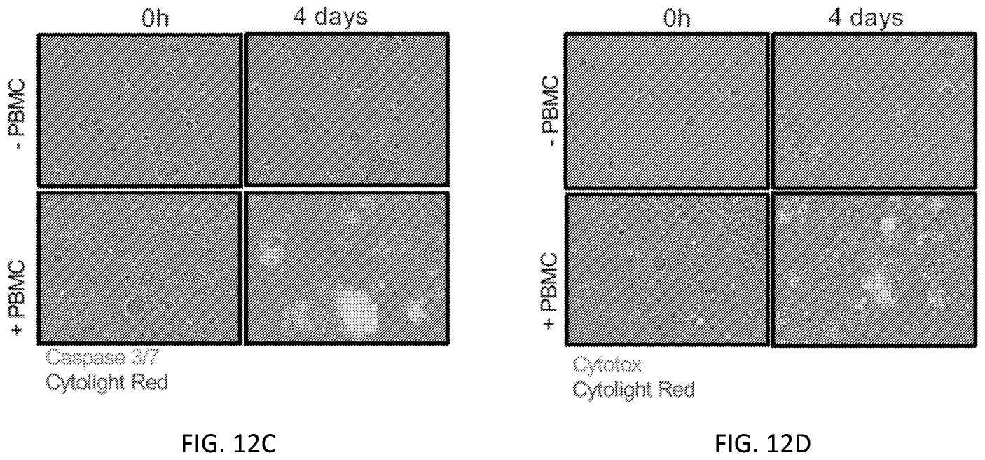

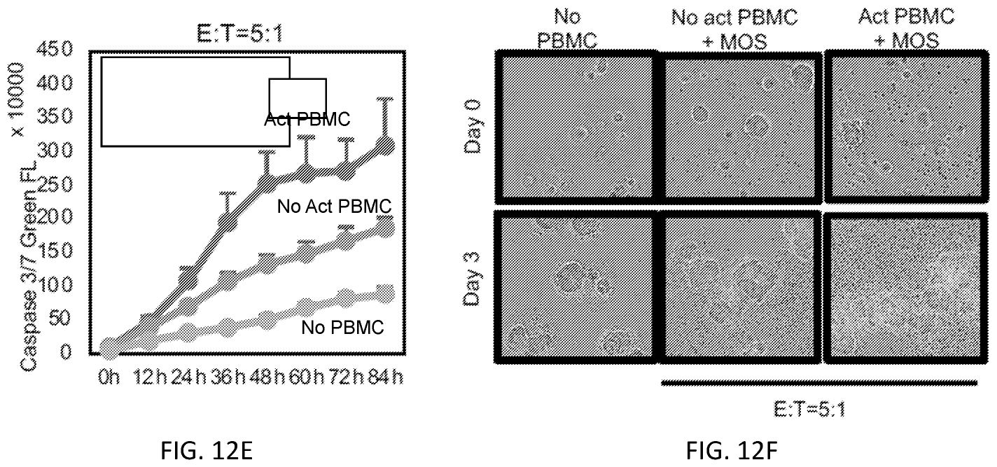

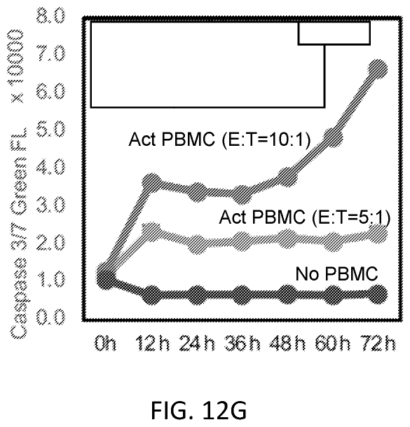

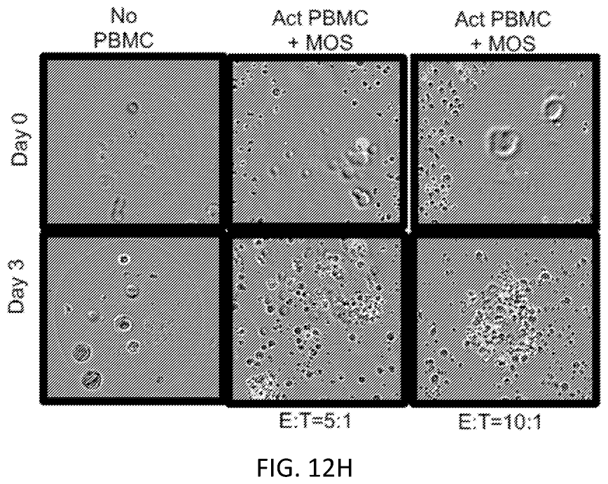

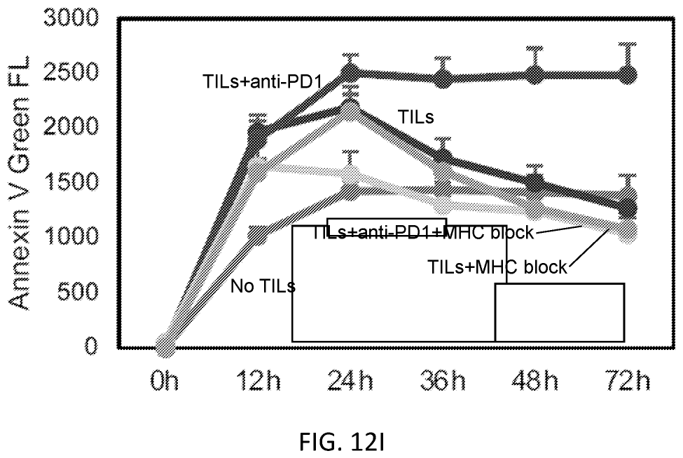

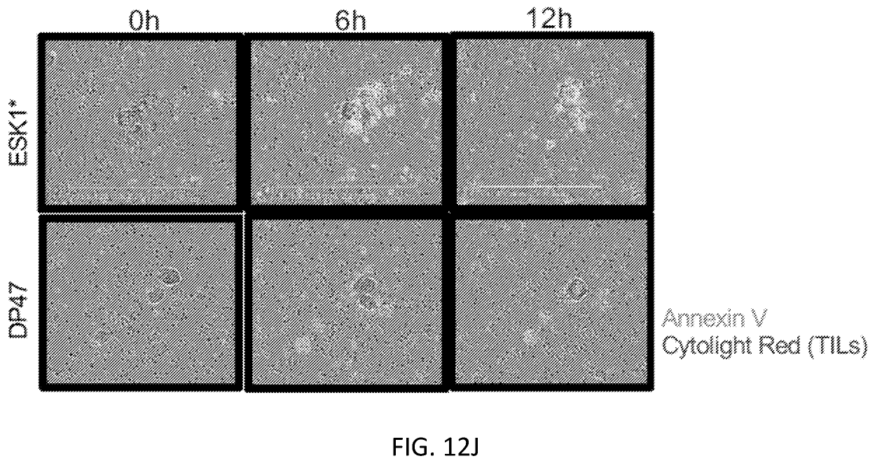

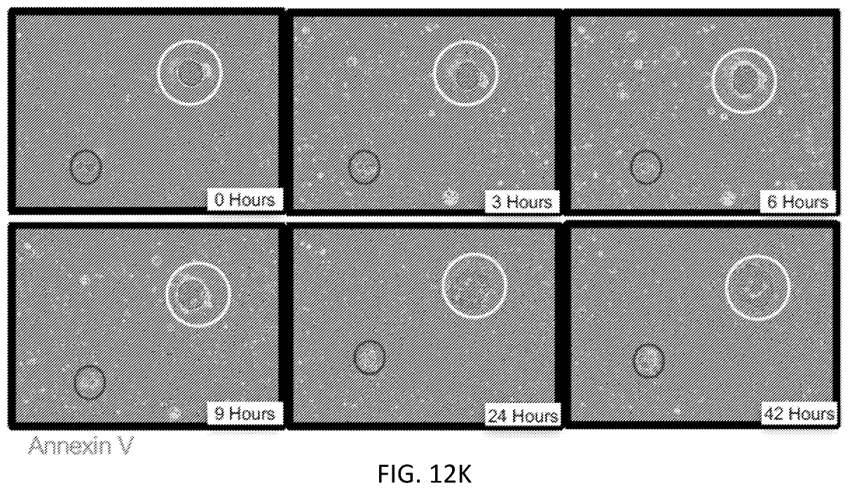

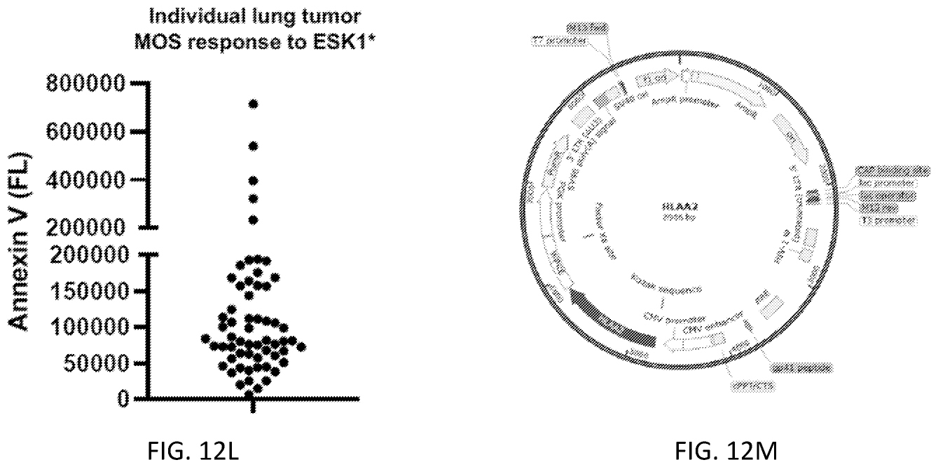

To explore whether MOS can be used as a potential potency assay, penetration of autologous patient-derived TILs into bulk MATRIGEL® vs. MOS (20 cells per MOS) was assessed. Most T cells stayed on the periphery of the bulk MATRIGEL® gel. Conversely, T cells readily infiltrated into MOS (due to their smaller size and larger surface-to-volume ratio) and adhered to tumor cells (FIGS. 11A and 11B). Peripheral blood mononuclear cell (PBMC) penetration into MATRIGEL® vs. MOS (20 cells per MOS) also was compared using the Incucyte live imaging system, revealing that immune cells could easily infiltrate MOS (FIG. 12A). Time-lapse fluorescence imaging was used to measure immune cytotoxicity against target tumor cells with TILs and PBMCs. For the TIL potency assay, MOS generated at density of 30 tumor cells per MOS were grown simultaneously with TILs from the same lung tumor tissue. Increased killing (indicated by Annexin V) was observed in MOS treated with autologous TILs (FIGS. 11C and 11D). This assay confirmed the potency of rapid expansion protocol (REP) TILs against matched lung tumor MOS, thus providing promising preliminary data as a TIL potency assay.

The potency of PBMCs against lung tumor MOS was then assessed to demonstrate that MOS can be used as an in vitro platform for cell therapy. MOS were derived from lung cancer patients, and allogeneic PBMCs from a different normal patient were added. Tumorspheres within MOS remained viable, appearing orange as labeled by Cytolight Rapid Red, after 96 hours of co-culture with PBMCs. However, when PBMCs were activated by anti-CD3 and anti-CD28 antibodies, the tumorspheres exhibited increased cell death as shown by Annexin V staining (FIG. 12B).

The response of lung cancer MOS (20 cells per MOS) to activated PBMC was characterized using Annexin V (early-stage cell surface apoptosis), Caspase 3/7 (enzyme-mediated cell apoptosis), and Cytotox (cell membrane integrity). PBMCs were pre-stained with live cell marker Cytolight Red dye. Both Annexin V and Caspase 3/7 could detect MOS apoptosis, while Caspase 3/7 had higher specificity (FIGS. 11E, 11F, 12C, and 12D). An imaging analysis pipeline was developed to identify MOS area to mask out background noise from outside immune cells (FIG. 11G), which confirmed PBMC-induced MOS apoptosis with less background signal from outside the MOS (FIG. 11H). PBMCs also induced tumorsphere death in CRC MOS (20 cells per MOS), which was enhanced by cytokine activation (FIGS. 12E and 12F), and in kidney cancer MOS (20 cells per MOS), which was enhanced by higher effector: target cell ratio (FIGS. 12G and 12H).

Adjunctive therapies were explored by first combining PD-1 blockade (nivolumab) with autologous TILs against matched lung tumor MOS. PD-1 blockade enhanced TIL-mediated killing inside MOS, which was abrogated by blocking MHC (FIG. 12I). TCB were then combined with autologous TILs or allogeneic PBMCs to treat lung cancer MOS (20 cells per MOS) expressing HLA-A2. ESK1* enhanced both TIL- and PBMC-induced tumor cell death compared to DP47 (FIGS. 11I, 11J, 12J, and 12K). Annexin V signals were higher in MOS treated with ESK1* vs. DP47 in all seven lung cancer samples (FIG. 11K). As a negative control, ESK1* did not enhance killing of HLA-A2 (−) lung cancer MOS, as indicated by the red arrow. Heterogeneity in drug response between MOS from the same patient was observed and quantified (FIGS. 12K and 12L).

Conventional bulk organoids require single cell dissociation for viral gene delivery before re-embedding into MATRIGEL®. Due to their small size and large surface-to-volume ratio, MOS can be infected by directly adding lentiviruses into culture medium without dissociation. This provides a convenient way to edit MOS at passage 0. Lung cancer MOS (20 cells per MOS) from an HLA-A2 (−) patient was infected with a lentiviral HLA-A2 expression vector for 3 days with dsRed as a control (FIGS. 11L and 12M). The infected MOS showed high expression of HLA-A2 (FIGS. 11M and 11N). Combinatorial treatments were then performed with ESK1* and activated PBMCs on HLA-A2-infected MOS. HLA-A2-infected MOS underwent more cell death than matched uninfected MOS in the presence of ESK1* and activated PBMCs, thus validating that HLA-A2 expression level mediates the efficacy of ESK1*+PBMC treatment (FIG. 11O).

TABLE 1
CRC patient demographics and clinical diagnosis

| | | | | | | Primary | Metastatic | |
|---|---|---|---|---|---|---|---|---|
| ID | Age | Gender | Race | Histology | Grade | site | site | Molecular profiling |
| CRC-MOS-001 | 87 | F | Caucasian | Adenocarcinoma | Moderately | Rectal | Lung | MSS |
| | | | | | differentiated | | | |
| CRC-MOS-002 | 68 | M | Caucasian | Adenocarcinoma | Moderately | Colon | Liver | MSS, TMB 1 Muts/Mb, |
| | | | | | differentiated | | | KRAS (G12V), APC |
| | | | | | | | | (L674fs), p53 (I254S) |
| CRC-MOS-003 | 71 | M | Caucasian | Adenocarcinoma | Moderately | Colon | Liver | MSS, TMB 3 Mut/Mb, |
| | | | | | differentiated | | | KRAS (G12V), APC |
| | | | | | | | | (Q1303), SMAD4 loss, |
| | | | | | | | | TP53 (L257Q) |
| CRC-MOS-004 | 62 | F | Caucasian | Adenocarcinoma | Moderately | Colon | Liver | MSS, TMB 3 Mut/Mb, |
| | | | | | differentiated | | | KRAS WT |
| | | | | | | | | APC (R1450), ATM |
| | | | | | | | | (R805), PIK3CA (E545K) |
| CRC-MOS-005 | 73 | M | Caucasian | Adenocarcinoma | Moderately | Colon | Liver | MSS, TMB 4 Mut/Mb, |
| | | | | | differentiated | | | KRAS (G12D), APC |
| | | | | | | | | (R876), TP53 (R158fs), |
| | | | | | | | | PIK3CA (E545Q) |
| CRC-MOS-006 | 31 | F | Asian | Adenocarcinoma | Poorly | Rectal | Pelvis | MSS, TMB (4 Muts/Mb) |
| | | | | | differentiated | | | KRAS (G12D), TP53 |
| | | | | | | | | (R175H) |
| CRC-MOS-007 | 37 | M | Caucasian | Adenocarcinoma | | Colon | Liver | MSS, TMB (1 Mut/Mb), |
| | | | | | | | | KRAS (G12D), APC |
| | | | | | | | | (R216), TP53 (G226E) |
| CRC-MOS-008 | 68 | M | Caucasian | Adenocarcinoma | Moderately | Colon | Liver | MSS, TMB (6 Mut/Mb) |
| | | | | | differentiated | | | KRAS WT, APC |
| | | | | | | | | (Q1367), EGFR |
| | | | | | | | | amplification |
| | | | | | | | | TP53 (R248W) |

TABLE 2
MOS correlation to CRC patient outcome

| | MOS | Clinical | Response | | Drug | |
|---|---|---|---|---|---|---|
| Patient ID | Prediction | Outcome | Time | | Screen | |
| CRC-MOS-001 | Sensitive | Response | 46 | wks | 8 | days |
| CRC-MOS-002 | Resistance | Response | 28 | wks | 9 | days |
| CRC-MOS-003 | Sensitive | Response | 38 | wks | 12 | days |
| CRC-MOS-004 | Resistance | No Response | 8 | wks | 8 | days |
| CRC-MOS-005 | Sensitive | Response | 36 | wks | 8 | days |
| CRC-MOS-006 | Resistance | No Response | 3 | wks | 10 | days |
| CRC-MOS-007 | Sensitive | No Response | 24 | wks | 11 | days |
| CRC-MOS-008 | Resistance | No Response | 6 | wks | 13 | days |

TABLE 3
Overview of microfluidic-based MOS device

| Device Highlights | | Off the shelf parts | |
|---|---|---|---|
| | Two samples | System | 1.7 Cu. Ft. Mini |
| Throughput | simultaneously | cooling | fridge |
| MOS size | 250-300 μM | Imaging | Compact Scientific |
| range | diameter | (ThorLabs | 1.3 MP Mono Camera, |
| | | | USB3.0 (#CS135MU) |
| | | | 6X Zoom-6.50X Zoom |
| | | | Lens with 12 mm Fine |
| | | | Focus (#MVL6X12Z) |
| | | | Machine Vision C-Mount |
| | | | Coupler (#MVLCMC) |
| | | | 1.00X Adapter |
| | | | (#MVL10A) |
| Sample | 10-1000 μl | Pressure | Fluigent Flow EZ |
| volume | | Control | pressure controllers |
| range | | | Fluigen P-cap(15 mL |
| | | | Falcon and 2 mL |
| | | | Eppendorf) |
| MOS | 2000 MOS for | Chip and | Customized |
| generation | biopsy (45 sec) | manifold | |
| rate | 1M MOS for high | | |
| | throughput drug | | |
| | screening (120 | | |
| | min) | | |
| MOS | Biological | | |
| MATRIGEL ® | hydrogel | | |
| Reagents | Bulk storage | | |

TABLE 4
Tumor patient demographics and clinical histology diagnosis

| Patient ID | Age | Gender | Race | Histology | Primary site | Metastatic site |
|---|---|---|---|---|---|---|
| 20-433 | 18 | F | Caucasian | Fibroadenoma negative for malignancy | Breast | |
| 20-ADU | 15 | F | Caucasian | Fibroadenoma negative for atypia and | Breast | |
| | | | | malignancy | | |
| 20-522 | 24 | F | Caucasian | Fibroadenoma negative for malignancy | Breast | |
| 20-489 | 33 | F | Asian | Complex fibroadenoma negative for atypia | Breast | |
| | | | | or carcinoma | | |
| 20-466 | 65 | M | African American | Clear cell renal carcinoma | Kidney | |
| 20-516 | 76 | F | African American | Clear cell renal carcinoma | Kidney | |
| 20-453 | 71 | F | Caucasian | Clear cell papillary renal carcinoma | Kidney | |
| 20-506 | 40 | F | Caucasian | Metastatic ovarian cancer with peritoneal | Ovary | Peritoneal cavity |
| | | | | carcinomatosis | | |
| 20-605 | 46 | F | Caucasian | Adenocarcinoma of lower lobe of left lung | Lung | Nodal lymphovascular spaces |
| 20-532 | 76 | F | Caucasian | Squamous cell carcinoma of lung | Lung | Lymph nodes |
| 20-500 | 64 | F | Caucasian | Adenocarcinoma of the lung, lower left lobe | Lung | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
obtaining a plurality of cells derived from a tissue;
forming droplets from the plurality of cells;
culturing the droplets; and
introducing a virus into the droplet culture, thereby obtaining one or more cells infected with the virus in the droplet.

2. The method of claim 1, wherein the one or more infected cells express one or more genes introduced by the virus after infection with the virus.

3. The method of claim 1, wherein the droplets have an average diameter of about 50 μm to about 500 μm.

4. The method of claim 1, wherein the plurality of cells includes no more than 15,000 cells.

5. The method of claim 1, wherein the cells are derived from a biopsy.

6. The method of claim 1, wherein the cells are derived from a tumor biopsy.

7. The method of claim 1, wherein the cells are derived from one or more core biopsies comprising from about a 14-gauge core to about a 20-gauge core biopsy.

8. The method of claim 1, wherein the cells are derived from one or more 18-gauge core biopsies.

9. The method of claim 1, wherein the cells are derived from a tumor biopsy for one or more cancers.

10. The method of claim 9, wherein the one or more cancers comprise rectal cancer, lung cancer, breast cancer, colorectal cancer (CRC), kidney cancer, ovarian cancer, or combinations thereof.

11. The method of claim 1, wherein the cells are derived from one or more patients.

12. The method of claim 1, wherein the cells comprise CRC patient-derived xenograft (PDX) cells.

13. The method of claim 1, wherein the droplets comprise tumorspheres.

14. The method of claim 1, wherein nascent droplets include a seeding density of about 1 to about 300 cells per droplet.

15. The method of claim 1, wherein nascent droplets include a seeding density configured to generate tumorspheres in the MOS of a desired quantity, size, or both.

16. The method of claim 1, wherein the method further comprises determining a number of droplets by dividing a number of viable cells by a number of cells per droplet.

17. The method of claim 1, further comprising treating the droplets with one or more therapeutic agents.

18. The method of claim 17, wherein the one or more therapeutic agents comprise a small molecule or an antibody.

19. The method of claim 1, wherein the cells are from a patient, and wherein the droplets function as a predictive model of the patient's sensitivity to one or more drug therapies for treating a disease.

20. The method of claim 19, wherein the droplets function as a predictive model of the patient's sensitivity to one or more chemotherapies.
